SEQUENCE LISTING (185 sequences)

SEQ ID NO: 1 - protein, 16 aa, Artificial Sequence
  Description of Artificial Sequence Synthetic peptide
AKTTPKLEEGEFSEAR

SEQ ID NO: 2 - protein, 17 aa, Artificial Sequence
  Description of Artificial Sequence Synthetic peptide
AKTTPKLEEGEFSEARV

SEQ ID NO: 3 - protein, 9 aa, Artificial Sequence
  Description of Artificial Sequence Synthetic peptide
AKTTPKLGG

SEQ ID NO: 4 - protein, 10 aa, Artificial Sequence
  Description of Artificial Sequence Synthetic peptide
SAKTTPKLGG

SEQ ID NO: 5 - protein, 6 aa, Artificial Sequence
  Description of Artificial Sequence Synthetic peptide
SAKTTP

SEQ ID NO: 6 - protein, 6 aa, Artificial Sequence
  Description of Artificial Sequence Synthetic peptide
RADAAP

SEQ ID NO: 7 - protein, 9 aa, Artificial Sequence
  Description of Artificial Sequence Synthetic peptide
RADAAPTVS

SEQ ID NO: 8 - protein, 12 aa, Artificial Sequence
  Description of Artificial Sequence Synthetic peptide
RADAAAAGGPGS

SEQ ID NO: 9 - protein, 27 aa, Artificial Sequence
  Description of Artificial Sequence Synthetic peptide
RADAAAAGGGGSGGGGSGGGGSGGGGS

SEQ ID NO: 10 - protein, 18 aa, Artificial Sequence
  Description of Artificial Sequence Synthetic peptide
SAKTTPKLEEGEFSEARV

SEQ ID NO: 11 - protein, 5 aa, Artificial Sequence
  Description of Artificial Sequence Synthetic peptide
ADAAP

SEQ ID NO: 12 - protein, 12 aa, Artificial Sequence
  Description of Artificial Sequence Synthetic peptide
ADAAPTVSIFPP

SEQ ID NO: 13 - protein, 5 aa, Artificial Sequence
  Description of Artificial Sequence Synthetic peptide
TVAAP

SEQ ID NO: 14 - protein, 12 aa, Artificial Sequence
  Description of Artificial Sequence Synthetic peptide
TVAAPSVFIFPP

SEQ ID NO: 15 - protein, 6 aa, Artificial Sequence
  Description of Artificial Sequence Synthetic peptide
QPKAAP

SEQ ID NO: 16 - protein, 13 aa, Artificial Sequence
  Description of Artificial Sequence Synthetic peptide
QPKAAPSVTLFPP

SEQ ID NO: 17 - protein, 6 aa, Artificial Sequence
  Description of Artificial Sequence Synthetic peptide
AKTTPP

SEQ ID NO: 18 - protein, 13 aa, Artificial Sequence
  Description of Artificial Sequence Synthetic peptide
AKTTPPSVTPLAP

SEQ ID NO: 19 - protein, 6 aa, Artificial Sequence
  Description of Artificial Sequence Synthetic peptide
AKTTAP

SEQ ID NO: 20 - protein, 13 aa, Artificial Sequence
  Description of Artificial Sequence Synthetic peptide
AKTTAPSVYPLAP

SEQ ID NO: 21 - protein, 6 aa, Artificial Sequence
  Description of Artificial Sequence Synthetic peptide
ASTKGP

SEQ ID NO: 22 - protein, 13 aa, Artificial Sequence
  Description of Artificial Sequence Synthetic peptide
ASTKGPSVFPLAP

SEQ ID NO: 23 - protein, 15 aa, Artificial Sequence
  Description of Artificial Sequence Synthetic peptide
GGGGSGGGGSGGGGS

SEQ ID NO: 24 - protein, 15 aa, Artificial Sequence
  Description of Artificial Sequence Synthetic peptide
GENKVEYAPALMALS

SEQ ID NO: 25 - protein, 15 aa, Artificial Sequence
  Description of Artificial Sequence Synthetic peptide
GPAKELTPLKEAKVS

SEQ ID NO: 26 - protein, 15 aa, Artificial Sequence
  Description of Artificial Sequence Synthetic peptide
GHEAAAVMQVQYPAS

SEQ ID NO: 27 - protein, 24 aa, Artificial Sequence
  Description of Artificial Sequence Synthetic peptide
TVAAPSVFIFPPTVAAPSVFIFPP

SEQ ID NO: 28 - protein, 26 aa, Artificial Sequence
  Description of Artificial Sequence Synthetic peptide
ASTKGPSVFPLAPASTKGPSVFPLAP

SEQ ID NO: 29 - protein, 5 aa, Artificial Sequence
  Description of Artificial Sequence Synthetic peptide
GGGGS

SEQ ID NO: 30 - protein, 118 aa, Artificial Sequence
  Description of Artificial Sequence Synthetic polypeptide
EVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 31 - protein, 108 aa, Artificial Sequence
  Description of Artificial Sequence Synthetic polypeptide
AIQMTQSPSSLSASVGDRVTITCQASQDIGNWLAWYQQKPGKSPKLLIYGATSLADGVPSRFSGSRSGTDFTLTISSLQPEDFATYYCLQAYNTPWTFGGGTKVEIKR

SEQ ID NO: 32 - protein, 118 aa, Artificial Sequence; identical to SEQ ID NO: 30
  Description of Artificial Sequence Synthetic polypeptide

SEQ ID NO: 33 - protein, 108 aa, Artificial Sequence
  Description of Artificial Sequence Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 34 - protein, 118 aa, Artificial Sequence
  Description of Artificial Sequence Synthetic polypeptide
QVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSS

SEQ ID NO: 35 - protein, 108 aa, Artificial Sequence; identical to SEQ ID NO: 31
  Description of Artificial Sequence Synthetic polypeptide

SEQ ID NO: 36 - protein, 118 aa, Artificial Sequence; identical to SEQ ID NO: 34
  Description of Artificial Sequence Synthetic polypeptide

SEQ ID NO: 37 - protein, 108 aa, Artificial Sequence; identical to SEQ ID NO: 33
  Description of Artificial Sequence Synthetic polypeptide

SEQ ID NO: 38 - protein, 119 aa, Artificial Sequence [sequence omitted]

SEQ ID NO: 39 - protein, 113 aa, Artificial Sequence
  Description of Artificial Sequence Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 40 - protein, 243 aa, Artificial Sequence
  Description of Artificial Sequence Synthetic polypeptide
EVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSSASTKGPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 41 - protein, 226 aa, Artificial Sequence
  Description of Artificial Sequence Synthetic polypeptide
AIQMTQSPSSLSASVGDRVTITCQASQDIGNWLAWYQQKPGKSPKLLIYGATSLADGVPSRFSGSRSGTDFTLTISSLQPEDFATYYCLQAYNTPWTFGGGTKVEIKRTVAAPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 42 - protein, 243 aa, Artificial Sequence
  Description of Artificial Sequence Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPEVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 43 - protein, 226 aa, Artificial Sequence
  Description of Artificial Sequence Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPAIQMTQSPSSLSASVGDRVTITCQASQDIGNWLAWYQQKPGKSPKLLIYGATSLADGVPSRFSGSRSGTDFTLTISSLQPEDFATYYCLQAYNTPWTFGGGTKVEIKR

SEQ ID NO: 44 - protein, 243 aa, Artificial Sequence; identical to SEQ ID NO: 40
  Description of Artificial Sequence Synthetic polypeptide

SEQ ID NO: 45 - protein, 226 aa, Artificial Sequence
  Description of Artificial Sequence Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKRTVAAPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 46 - protein, 243 aa, Artificial Sequence; identical to SEQ ID NO: 42
  Description of Artificial Sequence Synthetic polypeptide

SEQ ID NO: 47 - protein, 226 aa, Artificial Sequence
  Description of Artificial Sequence Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPEIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 48 - protein, 243 aa, Artificial Sequence
  Description of Artificial Sequence Synthetic polypeptide
QVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSSASTKGPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 49 - protein, 226 aa, Artificial Sequence; identical to SEQ ID NO: 41
  Description of Artificial Sequence Synthetic polypeptide

SEQ ID NO: 50 - protein, 243 aa, Artificial Sequence
  Description of Artificial Sequence Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPQVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSS

SEQ ID NO: 51 - protein, 226 aa, Artificial Sequence; identical to SEQ ID NO: 43
  Description of Artificial Sequence Synthetic polypeptide

SEQ ID NO: 52 - protein, 243 aa, Artificial Sequence; identical to SEQ ID NO: 48
  Description of Artificial Sequence Synthetic polypeptide

SEQ ID NO: 53 - protein, 226 aa, Artificial Sequence; identical to SEQ ID NO: 45
  Description of Artificial Sequence Synthetic polypeptide

SEQ ID NO: 54 - protein, 243 aa, Artificial Sequence; identical to SEQ ID NO: 50
  Description of Artificial Sequence Synthetic polypeptide

SEQ ID NO: 55 - protein, 226 aa, Artificial Sequence; identical to SEQ ID NO: 47
  Description of Artificial Sequence Synthetic polypeptide

SEQ ID NO: 56 - protein, 118 aa, Artificial Sequence
  Description of Artificial Sequence Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 57 - protein, 108 aa, Artificial Sequence
  Description of Artificial Sequence Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 58 - protein, 120 aa, Artificial Sequence [sequence omitted]

SEQ ID NO: 59 - protein, 108 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 60 - protein, 243 aa, Artificial Sequence
  Description of Artificial Sequence Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 61 - protein, 226 aa, Artificial Sequence
  Description of Artificial Sequence Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 62 - protein, 250 aa, Artificial Sequence
  Description of Artificial Sequence Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 63 - protein, 233 aa, Artificial Sequence
  Description of Artificial Sequence Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPSVFIFPPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 64 - protein, 243 aa, Artificial Sequence
  Description of Artificial Sequence Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 65 - protein, 226 aa, Artificial Sequence
  Description of Artificial Sequence Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 66 - protein, 244 aa, Artificial Sequence
  Description of Artificial Sequence Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 67 - protein, 221 aa, Artificial Sequence
  Description of Artificial Sequence Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 68 - protein, 244 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 69 - protein, 221 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 70 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 250 aa, Artificial Sequence
  Description of Artificial Sequence Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPSVFPLAPEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 75 - protein, 226 aa, Artificial Sequence; identical to SEQ ID NO: 65
  Description of Artificial Sequence Synthetic polypeptide

SEQ ID NO: 76 - protein, 10 aa, Artificial Sequence
  Description of Artificial Sequence Synthetic peptide
GFTFSNYGMH

SEQ ID NO: 77 - protein, 17 aa, Artificial Sequence
  Description of Artificial Sequence Synthetic peptide
MIYYDSSKMNYADTVKG

SEQ ID NO: 78 - protein, 9 aa, Artificial Sequence
  Description of Artificial Sequence Synthetic peptide
PTSHYVVDV

SEQ ID NO: 79 - protein, 11 aa, Artificial Sequence
  Description of Artificial Sequence Synthetic peptide
QASQDIGNWLA

SEQ ID NO: 80 - protein, 7 aa, Artificial Sequence
  Description of Artificial Sequence Synthetic peptide
GATSLAD

SEQ ID NO: 81 - protein, 9 aa, Artificial Sequence
  Description of Artificial Sequence Synthetic peptide
LQAYNTPWT

SEQ ID NO: 82 - protein, 17 aa, Artificial Sequence
  Description of Artificial Sequence Synthetic peptide
MIYYDSSKMNYADTVKG

SEQ ID NO: 83 - protein, 9 aa, Artificial Sequence
  Description of Artificial Sequence Synthetic peptide
PTSHYVVDV

SEQ ID NO: 84 - protein, 11 aa, Artificial Sequence
  Description of Artificial Sequence Synthetic peptide
QASQDIGNWLA

SEQ ID NO: 85 - protein, 7 aa, Artificial Sequence
  Description of Artificial Sequence Synthetic peptide
GATSLAD

SEQ ID NO: 86 - protein, 9 aa, Artificial Sequence
  Description of Artificial Sequence Synthetic peptide
LQAYNTPWT

SEQ ID NO: 87 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 88 - protein, 118 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQFPTYLPTFGQGTKVEIKRDIQMTQSPSS

SEQ ID NO: 89 - protein, 118 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQGYNDPPTFGQGTKVEIKRDIQMTQSPSS

SEQ ID NO: 90 - protein, 118 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSSTDPTTFGQGTKVEIKRDIQMTQSPSS

SEQ ID NO: 91 - protein, 118 aa, Artificial Sequence
  Description of Artificial Sequence Synthetic polypeptide
DIQMTQSPSSLSASVGDRVTITCRASQVVANSLAWYQQKPGKAPKLLIYLASFLYSGVPSRFSGSGSGTDFTLTISSLQPEDFATYYCQQDATSPPTFGQGTKVEIKRDIQMTQSPSS

SEQ ID NO: 92 - protein, 118 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYATDPPTFGQGTKVEIKRDIQMTQSPSS

SEQ ID NO: 93 - protein, 119 aa, Artificial Sequence
  Description of Artificial Sequence Synthetic polypeptide
EVQLVESGGGLVQPGGSLRLSCAASGFTFSGYAIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 94 - protein, 119 aa, Artificial Sequence
  Description of Artificial Sequence Synthetic polypeptide
EVQLVESGGGLVQPGGSLRLSCAASGFTFLGYGIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 95 - protein, 119 aa, Artificial Sequence; identical to SEQ ID NO: 93
  Description of Artificial Sequence Synthetic polypeptide

SEQ ID NO: 96 - protein, 119 aa, Artificial Sequence; identical to SEQ ID NO: 93
  Description of Artificial Sequence Synthetic polypeptide

SEQ ID NO: 97 - protein, 119 aa, Artificial Sequence; identical to SEQ ID NO: 93
  Description of Artificial Sequence Synthetic polypeptide

SEQ ID NO: 98 - protein, 119 aa, Artificial Sequence; identical to SEQ ID NO: 93
  Description of Artificial Sequence Synthetic polypeptide

SEQ ID NO: 99 - protein, 112 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSS

SEQ ID NO: 100 - protein, 113 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 101 - protein, 126 aa, Artificial Sequence
  Description of Artificial Sequence Synthetic polypeptide
QVQLVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSS

SEQ ID NO: 102 - protein, 110 aa, Artificial Sequence
  Description of Artificial Sequence Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 103 - protein, 122 aa, Artificial Sequence
  Description of Artificial Sequence Synthetic polypeptide
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 104 - protein, 113 aa, Artificial Sequence
  Description of Artificial Sequence Synthetic polypeptide
QVQLVQSGAEVKKPGSSVKVSCKASGYTFTNYDIHWVRQAPGQGLEWMGWIYPGDGSTKYNEKFKGRVTITADESTSTAYMELSSLRSEDTAVYYCAREWAYWGQGTTVTVSS

SEQ ID NO: 105 - protein, 108 aa, Artificial Sequence
  Description of Artificial Sequence Synthetic polypeptide
DIQMTQSPSSLSASVGDRVTITCRASQDIGGNLYWYQQKPGKAPKLLIYATSSLDSGVPSRFSGSRSGTDYTLTISSLQPEDFATYYCLQYSSSPWTFGQGTKVEIKR

SEQ ID NO: 106 - protein, 108 aa, Artificial Sequence
  Description of Artificial Sequence Synthetic polypeptide
DIQMTQSPSSLSASVGDRVTITCRASQDIGGNLYWLQQKPGKAPKRLIYATSSLDSGVPKRFSGSRSGSDYTLTISSLQPEDFATYYCLQYSSSPWTFGQGTKVEIKR

SEQ ID NO: 107 - protein, 108 aa, Artificial Sequence
  Description of Artificial Sequence Synthetic polypeptide
DIQMTQSPSSLSASVGDRVTITCRASQDIGGNLYWLQQKPGKTIKRLIYATSSLDSGVPSRFSGSGSGTDYTLTISSLQPEDFATYYCLQYSSSPWTFGQGTKVEIKR

SEQ ID NO: 108 - protein, 108 aa, Artificial Sequence
  Description of Artificial Sequence Synthetic polypeptide
DIQMTQSPSSLSASVGDRVTITCRASQDIGGNLYWYQQKPGKAPKLLIYATSSLDSGVPSRFSGSGSGTDFTLTISSLQPEDFATYYCLQYSSSPWTFGQGTKVEIKR

SEQ ID NO: 109 - protein, 10 aa, Artificial Sequence
  Description of Artificial Sequence Synthetic peptide
GFTFSNYGMS

SEQ ID NO: 110 - protein, 17 aa, Artificial Sequence
  Description of Artificial Sequence Synthetic peptide
SIRSGGGRTYYSDNVKG

SEQ ID NO: 111 - protein, 10 aa, Artificial Sequence
  Description of Artificial Sequence Synthetic peptide
YDHYSGSSDY

SEQ ID NO: 112 - protein, 11 aa, Artificial Sequence
  Description of Artificial Sequence Synthetic peptide
KSSQSLLDSDG

SEQ ID NO: 113 - protein, 7 aa, Artificial Sequence
  Description of Artificial Sequence Synthetic peptide
LVSKLDS

SEQ ID NO: 114 - protein, 9 aa, Artificial Sequence
  Description of Artificial Sequence Synthetic peptide
WQGTHFPRT

SEQ ID NO: 115 - protein, 10 aa, Artificial Sequence
  Description of Artificial Sequence Synthetic peptide
GFTFSNYGMH

SEQ ID NO: 116 - protein, 17 aa, Artificial Sequence
  Description of Artificial Sequence Synthetic peptide
MIYYDSSKMNYADTVKG

SEQ ID NO: 117 - protein, 9 aa, Artificial Sequence
  Description of Artificial Sequence Synthetic peptide
PTSHYVVDV

SEQ ID NO: 118 - protein, 11 aa, Artificial Sequence
  Description of Artificial Sequence Synthetic peptide
QASQDIGNWLA

SEQ ID NO: 119 - protein, 7 aa, Artificial Sequence
  Description of Artificial Sequence Synthetic peptide
GATSLAD

SEQ ID NO: 120 - protein, 9 aa, Artificial Sequence
  Description of Artificial Sequence Synthetic peptide
LQAYNTPWT

SEQ ID NO: 121 - protein, 10 aa, Artificial Sequence
  Description of Artificial Sequence Synthetic peptide
GFNIKDTYIH

SEQ ID NO: 122 - protein, 17 aa, Artificial Sequence
  Description of Artificial Sequence Synthetic peptide
RIYPTNGYTRYADSVKG

SEQ ID NO: 123 - protein, 11 aa, Artificial Sequence
  Description of Artificial Sequence Synthetic peptide
WGGDGFYAMDY

SEQ ID NO: 124 - protein, 11 aa, Artificial Sequence
  Description of Artificial Sequence Synthetic peptide
RASQDVNTAVA

SEQ ID NO: 125 - protein, 7 aa, Artificial Sequence
  Description of Artificial Sequence Synthetic peptide
SASFLYS

SEQ ID NO: 126 - protein, 9 aa, Artificial Sequence
  Description of Artificial Sequence Synthetic peptide
QQHYTTPPT

SEQ ID NO: 127 - protein, 11 aa, Artificial Sequence
  Description of Artificial Sequence Synthetic peptide
RASQDVSTAVA

SEQ ID NO: 128 - protein, 7 aa, Artificial Sequence
  Description of Artificial Sequence Synthetic peptide
SASFLYS

SEQ ID NO: 129 - protein, 9 aa, Artificial Sequence
  Description of Artificial Sequence Synthetic peptide
QQSYTTPPT

SEQ ID NO: 130 - protein, 9 aa, Artificial Sequence
  Description of Artificial Sequence Synthetic peptide
QQFPTYLPT

SEQ ID NO: 131 - protein, 9 aa, Artificial Sequence
  Description of Artificial Sequence Synthetic peptide
QQGYNDPPT

SEQ ID NO: 132 - protein, 9 aa, Artificial Sequence
  Description of Artificial Sequence Synthetic peptide
QQSSTDPTT

SEQ ID NO: 133 - protein, 11 aa, Artificial Sequence
  Description of Artificial Sequence Synthetic peptide
RASQVVANSLA

SEQ ID NO: 134 - protein, 10 aa, Artificial Sequence
  Description of Artificial Sequence Synthetic peptide
QQDATSPPTF

SEQ ID NO: 135 - protein, 9 aa, Artificial Sequence
  Description of Artificial Sequence Synthetic peptide
QQYATDPPT

SEQ ID NO: 136 - protein, 10 aa, Artificial Sequence
  Description of Artificial Sequence Synthetic peptide
GFTFSGYAIH

SEQ ID NO: 137 - protein, 18 aa, Artificial Sequence
  Description of Artificial Sequence Synthetic peptide
GWISPAGGSTDYADSVKG

SEQ ID NO: 138 - protein, 10 aa, Artificial Sequence
  Description of Artificial Sequence Synthetic peptide
GPFSPWVMDY

SEQ ID NO: 139 - protein, 10 aa, Artificial Sequence
  Description of Artificial Sequence Synthetic peptide
GFTFLGYGIH

SEQ ID NO: 140 - protein, 10 aa, Artificial Sequence
  Description of Artificial Sequence Synthetic peptide
GFTFSGYAIH

SEQ ID NO: 141 - protein, 10 aa, Artificial Sequence
  Description of Artificial Sequence Synthetic peptide
GFTFSRYSMS

SEQ ID NO: 142 - protein, 17 aa, Artificial Sequence
  Description of Artificial Sequence Synthetic peptide
QINSVGNSTYYPDTVKG

SEQ ID NO: 143 - protein, 3 aa, Artificial Sequence
  Description of Artificial Sequence Synthetic peptide
GDY

SEQ ID NO: 144 - protein, 16 aa, Artificial Sequence
  Description of Artificial Sequence Synthetic peptide
RSSQSLIYSDGNAYLH

SEQ ID NO: 145 - protein, 7 aa, Artificial Sequence
  Description of Artificial Sequence Synthetic peptide
KVSNRFS

SEQ ID NO: 146 - protein, 9 aa, Artificial Sequence
  Description of Artificial Sequence Synthetic peptide
SQSTHVPWT

SEQ ID NO: 147 - protein, 10 aa, Artificial Sequence
  Description of Artificial Sequence Synthetic peptide
GFTFSSYAMS

SEQ ID NO: 148 - protein, 12 aa, Artificial Sequence
  Description of Artificial Sequence Synthetic peptide
AINASGTRTYYA

SEQ ID NO: 149 - protein, 17 aa, Artificial Sequence
  Description of Artificial Sequence Synthetic peptide
GKGNTHKPYGYVRYFDV

SEQ ID NO: 150 - protein, 12 aa, Artificial Sequence
  Description of Artificial Sequence Synthetic peptide
RASQSVSSSYLA

SEQ ID NO: 151 - protein, 7 aa, Artificial Sequence
  Description of Artificial Sequence Synthetic peptide
GASSRAT

SEQ ID NO: 152 - protein, 9 aa, Artificial Sequence
  Description of Artificial Sequence Synthetic peptide
LQIYNMPIT

SEQ ID NO: 153 - protein, 10 aa, Artificial Sequence
  Description of Artificial Sequence Synthetic peptide
GFKITHYTMG

SEQ ID NO: 154 - protein, 17 aa, Artificial Sequence
  Description of Artificial Sequence Synthetic peptide
RITWGGDNTFYSNSVKG

SEQ ID NO: 155 - protein, 13 aa, Artificial Sequence
  Description of Artificial Sequence Synthetic peptide
GSTSTATPLRVDY

SEQ ID NO: 156 - protein, 10 aa, Artificial Sequence
  Description of Artificial Sequence Synthetic peptide
GYTFTNYDIH

SEQ ID NO: 157 - protein, 17 aa, Artificial Sequence
  Description of Artificial Sequence Synthetic peptide
WIYPGDGSTKYNEKFKG

SEQ ID NO: 158 - protein, 8 aa, Artificial Sequence
  Description of Artificial Sequence Synthetic peptide
YWGQGTTV

SEQ ID NO: 159 - protein, 11 aa, Artificial Sequence
  Description of Artificial Sequence Synthetic peptide
RASQDIGGNLY

SEQ ID NO: 160 - protein, 7 aa, Artificial Sequence
  Description of Artificial Sequence Synthetic peptide
ATSSLDS

SEQ ID NO: 161 - protein, 9 aa, Artificial Sequence
  Description of Artificial Sequence Synthetic peptide
LQYSSSPWT

SEQ ID NO: 162 - protein, 121 aa, Artificial Sequence
  Description of Artificial Sequence Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSS

SEQ ID NO: 163 - protein, 107 aa, Artificial Sequence
  Description of Artificial Sequence Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKR

SEQ ID NO: 164 - protein, 5 aa, Artificial Sequence
  Description of Artificial Sequence Synthetic peptide
DYNMN

SEQ ID NO: 165 - protein, 17 aa, Artificial Sequence
  Description of Artificial Sequence Synthetic peptide
VINPNYGSSTYNQKFKG

SEQ ID NO: 166 - protein, 11 aa, Artificial Sequence
  Description of Artificial Sequence Synthetic peptide
KWGQLGRGFFD

SEQ ID NO: 167 - protein, 10 aa, Artificial Sequence
  Description of Artificial Sequence Synthetic peptide
RASSSVSYMH

SEQ ID NO: 168 - protein, 7 aa, Artificial Sequence
  Description of Artificial Sequence Synthetic peptide
ATSNLAS

SEQ ID NO: 169 - protein, 9 aa, Artificial Sequence
  Description of Artificial Sequence Synthetic peptide
QQWSSSPLT

SEQ ID NO: 170 - protein, 120 aa, Artificial Sequence
  Description of Artificial Sequence Synthetic polypeptide
EVQLVQSGAEVKKPGASVKVSCKASGYTFTSHGISWVRQAPGQGLDWMGWISPYSGNTNYAQKLQGRVTMTTDTSTSTAYMELSSLRSEDTAVYYCARVGSGPYYYMDVWGQGTLVTVSS

SEQ ID NO: 171 - protein, 110 aa, Artificial Sequence
  Description of Artificial Sequence Synthetic polypeptide
QSALTQPRSVSGSPGQSVTISCTGTSSSVGDSIYVSWYQQHPGKAPKLMLYDVTKRPSGVPDRFSGSKSGNTASLTISGLQAEDEADYYCCSYAGTDTLFGGGTKVTVLG

SEQ ID NO: 172 - protein, 5 aa, Artificial Sequence
  Description of Artificial Sequence Synthetic peptide
SHGIS

SEQ ID NO: 173 - protein, 16 aa, Artificial Sequence
  Description of Artificial Sequence Synthetic peptide
WISPYSGNTNYAQKLQ

SEQ ID NO: 174 - protein, 11 aa, Artificial Sequence
  Description of Artificial Sequence Synthetic peptide
VGSGPYYYMDV

SEQ ID NO: 175 - protein, 14 aa, Artificial Sequence
  Description of Artificial Sequence Synthetic peptide
TGTSSSVGDSIYVS

SEQ ID NO: 176 - protein, 7 aa, Artificial Sequence
  Description of Artificial Sequence Synthetic peptide
DVTKRPS

SEQ ID NO: 177 - protein, 9 aa, Artificial Sequence
  Description of Artificial Sequence Synthetic peptide
CSYAGTDTL

SEQ ID NO: 178 - protein, 10 aa, Artificial Sequence
  Description of Artificial Sequence Synthetic peptide
GGGGSGGGGS

SEQ ID NO: 179 - protein, 9 aa, Artificial Sequence
  Description of Artificial Sequence Synthetic peptide
GGSGGGGSG

SEQ ID NO: 180 - protein, 249 aa, Artificial Sequence
  Description of Artificial Sequence Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 181 - protein, 224 aa, Artificial Sequence
  Description of Artificial Sequence Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKRGGSGGGGSGDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 182 - protein, 245 aa, Artificial Sequence
  Description of Artificial Sequence Synthetic polypeptide
QVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSSASTKGPEFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSS

SEQ ID NO: 183 - protein, 227 aa, Artificial Sequence
  Description of Artificial Sequence Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKRTVAAPSVFIFPPQIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKR

SEQ ID NO: 184 - protein, 248 aa, Artificial Sequence
  Description of Artificial Sequence Synthetic polypeptide
EVQLVQSGAEVKKPGASVKVSCKASGYTFTSHGISWVRQAPGQGLDWMGWISPYSGNTNYAQKLQGRVTMTTDTSTSTAYMELSSLRSEDTAVYYCARVGSGPYYYMDVWGQGTLVTVSSGGGGSGGGGSEVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 185 - protein, 227 aa, Artificial Sequence
  Description of Artificial Sequence Synthetic polypeptide
QSALTQPRSVSGSPGQSVTISCTGTSSSVGDSIYVSWYQQHPGKAPKLMLYDVTKRPSGVPDRFSGSKSGNTASLTISGLQAEDEADYYCCSYAGTDTLFGGGTKVTVLGGGSGGGGSGEIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

(19) United States
(12) Patent Application Publication
(10) Pub. No.: US 2014/0212423 A1
(43) Pub. Date: Jul. 31, 2014

(54) BLOOD-BRAIN BARRIER PENETRATING DUAL SPECIFIC BINDING PROTEINS

(71) Applicants: Denise KARAOGLU HANZATIAN, Natick, MA (US); Tariq GHAYUR, Holliston, MA (US); Annette J. SCHWARTZ STERMAN, Princeton, MA (US); Andrew GOODEARL, Sherborn, MA (US); Maria Cristina HARRIS, Worcester, MA (US)

(72) Inventors: Denise KARAOGLU HANZATIAN, Natick, MA (US); Tariq GHAYUR, Holliston, MA (US); Annette J. SCHWARTZ STERMAN, Princeton, MA (US); Andrew GOODEARL, Sherborn, MA (US); Maria Cristina HARRIS, Worcester, MA (US)

(73) Assignee: ABBVIE, INC., Worcester, MA (US)

(21) Appl. No.: 14/097,033

(22) Filed: Dec. 4, 2013

Related U.S. Application Data
(60) Provisional application No. 61/792,163, filed on Mar. 15, 2013.
(60) Provisional application No. 61/733,252, filed on Dec. 4, 2012.

(51) Int. Cl. C07K 16/28

(52) U.S. Cl. CPC C07K 16/28
USPC 424/136.1; 530/387.3; 530/387.1; 536/23.53; 435/69.6

(57) ABSTRACT

Engineered multivalent and multispecific binding proteins capable of penetrating the blood-brain barrier (BBB) are provided, along with methods of making and uses in the prevention, diagnosis, and/or treatment of disease.

### RELATED APPLICATIONS

This international application claims priority from U.S. Provisional Application No. 61/733,252 filed Dec. 4, 2012 and U.S. Provisional Application No. 61/792,163 filed Mar. 15, 2013, the contents of which are hereby incorporated by reference in their entireties.

### FIELD

Multivalent and multispecific binding proteins that bind receptors on the Blood-Brain Barrier (BBB), and capable of carrying another brain target domain into the brain, methods of making, in vivo distribution in brain, and their uses in the treatment of acute and chronic neurological diseases, brain cancer, pain are provided.

### BACKGROUND

The brain prevents the passage of antibodies and other biologic drugs from the blood stream into the brain parenchyma by a highly vascular barrier system referred to as the blood-brain barrier (BBB). Therefore one of the most important factors for successful CNS drug development is the ability to effectively deliver such drugs to the brain. The poor distribution of antibody to the brain may be explained, in part, by inefficient convective uptake into the brain (e.g., because of the “tight junctions” in the brain vascular endothelium) and by rapid turnover of brain interstitial fluids, which would correlate with efficient convective elimination of IgG from the brain. As size, shape, lipophilicity and charge etc. are important parameters governing brain penetration, only approximately 0.1% of protein in blood gains access to the CNS through passive diffusion, although published values range from as low as 0.01% to as high as 0.4% (Bergman et al., 1998; Shen et al., 2004; Levites et al., 2006; Garg and Balthasar, 2009; Braen et al., 2010). Virtually identical IgG brain-to-plasma exposure ratios were observed following an intravenous (IV) dose of a murine monoclonal IgG1 antibody in wild-type animals and in FcRn-deficient mice (i.e., 0.0022±0.00015 vs. 0.0021±0.00011, P=0.3347, A. Garg and J. P. Balthasar). Mechanism and rates of IgG transport in the CNS are largely unknown.

A variety of drug delivery approaches have been employed to address the blood brain barrier (BBB) problem. Many of these approaches exploit the transport capabilities of specific receptors expressed on the brain vascular epithelium to transport a biologic across the BBB into the brain. These receptors can also be exploited to mediate the transfer of nanoparticles across the BBB. For example, nanoparticles coupled to receptor-targeting MAbs have been used to transport loperamide across the BBB.

In general, however, there remain several limitations associated with existing BBB delivery technology. For example, the transport kinetics and structural binding requirements of the transporter may necessitate modifications to the biologic in order to facilitate binding. Studies with an anti-receptor MAbs have shown that the affinity of the MAb to the receptor is of great importance to ensure dissociation of the MAb after receptor-mediated uptake. Additionally, these receptors show a widespread expression on peripheral organs as well which limits their application for brain-specific delivery. Finally, the molecular weight of the co-transported biologic must be of relatively low molecular weight to ensure uptake. Accordingly, there remains an urgent need for improved BBB delivery technologies, especially with regard to engineered binding proteins of high molecular weight that can penetrate into the brain without disrupting the blood-brain barrier. Such high molecular weight binding proteins include bispecific antibodies and other multivalent and multispecific binding proteins capable of binding two or more antigens (see PCT Publication No. WO 0177342 and U.S. Pat. No. 7,612,181).

### SUMMARY

This instant disclosure improves upon the art by providing high molecular weight (HMW) binding proteins capable of binding a BBB antigen (e.g., an extracellular receptor, a surface protein, an intracellular receptor, an intracellular protein, a carbohydrate, a target, and a ligand receptor) expressed on the brain vascular epithelium and traversing the BBB of a subject. In certain aspects, the disclosure provides HMW multivalent binding proteins (e.g., a DVD-Ig™) comprising at least one binding domain or binding site targeting an antigen (e.g., a transport receptor) combined with one or more second binding domains (e.g., variable domains) directed against a therapeutically relevant target. In addition, unlike other binding proteins, the binding proteins of the disclosure have one or more binding sites or domains (e.g., one, two, or three binding sites) which are unoccupied upon BBB uptake such that they remain available for binding to the therapeutically relevant target molecule present in the brain. Additionally or alternatively, one or more of the binding sites may be pre-loaded with a therapeutic agent (e.g., an endogenous or exogenous therapeutic protein) to facilitate delivery of the agent to brain. Accordingly, the binding proteins of the invention well-suited for the treatment of brain and CNS diseases including, but not limited to, Alzheimer's disease, Parkinson's disease, pain, epilepsy schizophrenia, and brain cancer. In particular, the feasibility of systemic delivery of DVD-Ig™ to brain tissue has been established by a variety of assays including brain immunohistochemistry.

In certain embodiments, the disclosure provides a dual variable domain (DVD) binding protein that specifically binds to an antigen expressed on brain vascular epithelium of a subject and facilitates uptake of a composition into the brain of the subject. In certain embodiments, the disclosure provides a binding protein that specifically binds to a receptor expressed on brain vascular epithelium of a subject.

In certain embodiments, the receptor expressed on brain vascular epithelium of a subject is selected from the group consisting of insulin receptor, transferrin receptor, LRP family receptor, melanocortin receptor, nicotinic acetylcholine receptor, VACM-1 receptor, vascular endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, S1P1, P2Y receptor, M6PR, Neuronal nicotinic acetylcholine receptor, Lipoprotein receptor, AchR, DTr, Glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, TMEM30A and RAGE, a low density lipoprotein receptor-related protein (LRP) family receptors include for example LRP1, LRP1b, LRP2, VLDL receptor, LRP4 and LRP8. Optionally, LRP family receptors can be chosen from LRP2 or LRP8. In certain embodiments, the receptor is a transferrin receptor. The binding protein in various embodiments binds the antigen which comprises a target. For example, the target comprises transferrin receptor.

In an embodiment, the binding protein binds to the antigen with an EC₅₀ of between 3 and 30 nM. For example, the EC₅₀ is at least about 3 nM to about 10 nM, about 10 nM to about 15 nM, about 15 nM to about 20 nM, about 20 nM to about 25 nM, or about 25 nM to about 30 nM.

In certain embodiments, the DVD binding protein comprises a DVD-Ig. In various embodiments, the DVD binding protein is selected from the group consisting of a half-DVD-Ig, a scDVD-Ig, an fDVD-Ig, an rDVD-Ig, a pDVD-Ig, an mDVD-Ig and a coDVD-Ig. For example, the DVD-Ig is humanized.

In various embodiments, the binding protein shows a 1 fold to 10 fold increase in brain concentration in a mammalian subject when administered systemically to the mammalian subject when compared to a second binding protein of the same class as the binding protein that does not specifically bind to a antigen expressed on brain vascular epithelium. In various embodiments, the subject is administered intravenously or topically.

In certain embodiments, the binding protein comprises a plurality of binding proteins, for example the binding proteins are located in a mixture, solution, or composition. In certain embodiments, the binding protein localizes to brain parenchyma or neuronal cell bodies of mammalian subjects when the binding protein is administered to mammalian subjects.

In certain embodiment, a concentration of the binding protein in the brain of a mammalian subject 96 hours after systemic administration to the mammalian subject is greater than 1% of the concentration of the binding protein in the brain of a mammalian subject 24 hours after systemic administration to the mammalian subject. In certain embodiments, the systemic administration is selected from the group consisting of intravenous administration, subcutaneous administration and intraperitoneal administration. In various embodiments, the binding protein is formulated for use with an applicator. For example, the applicator is a syringe, a dropper, a patch, a membrane, or a mesh.

In certain embodiments, the binding protein specifically binds to an antigen expressed on the brain vascular epithelium of a mammalian subject. In various embodiments, the mammal is selected from the group consisting of mice, rats, gerbils, hamsters, rabbits, apes, monkeys, humans, dogs, cats, camels, llamas, cattle and horses.

In certain embodiments, the antigen bound by the binding protein comprises a receptor expressed on brain vascular epithelium of a subject is selected from the group consisting of an insulin receptor, a transferrin receptor, a LRP, a melanocortin receptor, a nicotinic acetylcholine receptor, a VACM-1 receptor, vascular, IGFR, EPCR, EGFR, TNFR, leptin receptor, M6PR, a lipoprotein receptor, NCAM, LIFR, LfR, MRP1, AchR, DTr, Glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, an endothelial growth factor receptor (EGFR) for example EGFR1, EGFR2 and EGFR3, a glucocorticoid receptor, an ionotropic glutamate receptor, a M3 receptor, an aryl hydrocarbon receptor, a GLUT-1, inositol-1,4,5-trisphosphate (IP3) receptor, a N-methyl-D-aspartate receptor, S1P1, P2Y receptor, TMEM30A, and RAGE.

In certain embodiments, the binding protein further comprises a composition such that the composition is co-administered with the binding protein. For example, the composition comprises a pharmaceutically acceptable carrier or buffer. In various embodiments, the binding protein and composition are not-covalently linked and are mixed or contacted with one another.

In certain embodiments, the composition is covalently bound to the binding protein. In certain embodiments, the composition is covalently bound to the binding protein by a linker.

In certain embodiments, the composition is selected from the group consisting of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody or agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1 RI, sIL-1RII, sIL-6R, an anti-inflammatory cytokine, IL-4, IL-10, IL-11, IL-13, TGFβ, and combinations thereof.

In certain embodiments, the binding protein comprises a polypeptide chain, such that the polypeptide chain comprises VD1-(X1)n-VD2-C-(X2)n, such that

VD1 is a first heavy chain variable domain;

VD2 is a second heavy chain variable domain;

C is a heavy chain constant domain;

X1 is a linker with the proviso that it is not CH1;

X2 is an Fc region;

(X1)n is (X1)0 or (X1)1;

(X2)n is (X2)0 or (X2)1; and

such that the binding protein specifically binds TfR or HIR; and

(a) VD1 or VD2 comprises three CDRs wherein at least one CDR comprises an amino acid sequence selected from the group consisting of SEQ ID NO: 76, 77, 78, 82, 83, 115-117 and 156-158;

(b) VD1 and VD2 independently comprise three CDRs wherein at least one CDR comprises an amino acid sequence selected from the group consisting of SEQ ID NO: 76, 77, 78, 82, 83, 115-117 and 156-158; or

(c) VD1 comprises three CDRs wherein at least one CDR comprises an amino acid sequence selected from the group consisting of SEQ ID NO: 76, 77, 78, 82, 83, 115-117 and 156-158, and VD2 comprises three CDRs wherein at least one CDR comprises an amino acid sequence selected from the group consisting of SEQ ID NO: 76, 77, 78, 82, 83, 115-117 and 156-158.

In certain embodiments of the binding protein,

(a) VD1 or VD2 comprises an amino acid sequence selected from the group consisting of SEQ ID NO: 30, 32, 34, 36, 56, and 104; or

(b) VD1 and VD2 independently comprise an amino acid sequence selected from the group consisting of SEQ ID NO: 30, 32, 34, 36, 56, and 104.

In certain embodiments, the binding protein includes a polypeptide chain, such that the polypeptide chain comprises VD1-(X1)n-VD2-C-(X2)n, wherein

VD1 is a first light chain variable domain;

VD2 is a second light chain variable domain;

C is a light chain constant domain;

X1 is a linker with the proviso that it is not CL;

X2 does not comprise an Fc region;

(X1)n is (X1)0 or (X1)1;

(X2)n is (X2)0 or (X2)1; and

wherein the binding protein specifically binds TfR or HIR; and

(a) VD1 or VD2 comprises three CDRs each, wherein at least one CDR comprises an amino acid sequences selected from the group consisting of SEQ ID NO: 79, 80, 81, 84, 85, 86, 118, 119, 120, 159, 160, and 161;

(b) VD1 and VD2 independently comprise three CDRs each comprising amino acid sequences selected from the group consisting of SEQ ID NO: 79, 80, 81, 84, 85, 86, 118, 119, 120, 159, 160, and 161; or

(c) VD1 comprises three CDRs each comprising amino acid sequences selected from the group consisting of SEQ ID NO: 79, 80, 81, 84, 85, 86, 118, 119, 120, 159, 160, and 161, and VD2 comprises three CDRs each comprising amino acid sequences selected from the group consisting of SEQ ID NO: 79, 80, 81, 84, 85, 86, 118, 119, 120, 159, 160, and 161.

In certain embodiments,

(a) VD1 or VD2 comprises an amino acid sequence selected from the group consisting of SEQ ID NO: 31, 33, 35, 37, 57, 105, 106, 107, and 108; or

(b) VD1 and VD2 independently comprise an amino acid sequence selected from the group consisting of SEQ ID NO: 31, 33, 35, 37, 57, 105, 106, 107, and 108.

In certain embodiments of the binding protein, (X1)n is (X1)0.

The binding protein in certain embodiments comprises first and second polypeptide chains, wherein the first polypeptide chain comprises a first VD1-(X1)n-VD2-C-(X2)n, wherein

VD1 is a first heavy chain variable domain;

VD2 is a second heavy chain variable domain;

C is a heavy chain constant domain;

X1 is a first linker;

X2 is an Fc region;

wherein the second polypeptide chain comprises a second VD1-(X1)n-VD2-C-(X2)n, wherein

VD1 is a first light chain variable domain;

VD2 is a second light chain variable domain;

C is a light chain constant domain;

X1 is a second linker;

X2 does not comprise an Fc region;

(X1)n is independently (X1)0 or (X1)1 and (X2)n is independently (X2)0 or (X2)1

wherein the first and second X1 linker are the same or different;

wherein the first X1 linker is not CH1 and/or the second X1 linker is not CL;

wherein the binding protein specifically binds TfR or HIR; and

(a) VD1 or VD2 heavy chain variable domain comprises three CDRs each, wherein at least one of the CDRs comprises an amino acid sequences selected from the group consisting of SEQ ID NO: 76, 77, 78, 82, 83, 115, 116, 117, 156, 157, and 158;

(b) VD1 and VD2 heavy chain variable domains independently comprise three CDRs each comprising amino acid sequences selected from the group consisting of SEQ ID NO: 76, 77, 78, 82, 83, 115, 116, 117, 156, 157, and 158; or

(c) VD1 heavy chain variable domain comprises three CDRs each comprising amino acid sequences selected from the group consisting of SEQ ID NO: 76, 77, 78, 82, 83, 115, 116, 117, 156, 157, and 158, and VD2 heavy chain variable domain comprises three CDRs each comprising amino acid sequences selected from the group consisting of SEQ ID NO: 76, 77, 78, 82, 83, 115, 116, 117, 156, 157, and 158;

and wherein

(a) VD1 or VD2 light chain variable domain comprises three CDRs each comprising amino acid sequences selected from the group consisting of SEQ ID NO: 79, 80, 81, 84, 85, 86, 118, 119, 120, 159, 160, and 161;

(b) VD1 and VD2 light chain variable domains independently comprise three CDRs each comprising amino acid sequences selected from the group consisting of SEQ ID NO: 79, 80, 81, 84, 85, 86, 118, 119, 120, 159, 160, and 161; or

(c) VD1 light chain variable domain comprises three CDRs each comprising amino acid sequences selected from the group consisting of SEQ ID NO: 79, 80, 81, 84, 85, 86, 118, 119, 120, 159, 160, and 161; and VD2 light chain variable domain comprises three CDRs each comprising amino acid sequences selected from the group consisting of SEQ ID NO: 79, 80, 81, 84, 85, 86, 118, 119, 120, 159, 160, and 161.

In certain embodiments,

(a) VD1 or VD2 heavy chain variable domain comprises an amino acid sequence selected from the group consisting of SEQ ID NO: 30, 32, 34, 36, 56, and 104; or

(b) VD1 and VD2 heavy chain variable domains independently comprise an amino acid sequence selected from the group consisting of SEQ ID NO: 30, 32, 34, 36, 56 and 104;

and wherein

(a) VD1 or VD2 light chain variable domain comprises an amino acid sequence selected from the group consisting of SEQ ID NO: 31, 33, 35, 37, 57, 105, 106, 107, and 108; or

(b) VD1 and VD2 light chain variable domains independently comprise an amino acid sequence selected from the group consisting of SEQ ID NO: 31, 33, 35, 37, 57, 105, 106, 107, and 108.

In certain embodiments, X1 is a peptide linker comprising an at least one amino acid sequence described herein. For example, the at one amino acid sequence is selected from a member of the group consisting of SEQ ID NOs 1-29, 178 and 179.

In certain embodiments, the Fc region comprises a variant sequence Fc region. In certain embodiments, the Fc region comprises an Fc region selected from the group consisting of IgG1, IgG2, IgG3, IgG4, IgA, IgM, IgE, and IgD. In various embodiments, the Fc region comprises a humanized sequence or a human sequence.

In certain embodiments, the binding protein comprises two first polypeptide chains and two second polypeptide chains.

In certain embodiments, the VD1 of the first polypeptide chain and the VD1 of the second polypeptide chain are from a different first and second parent antibody, respectively, or antigen binding portion thereof.

In certain embodiments, the VD2 of the first polypeptide chain and the VD2 of the second polypeptide chain are from a different first and second parent antibody, respectively, or antigen binding portion thereof. In certain embodiments, the first and the second parent antibodies bind different epitopes on the antigen.

In certain embodiments, the binding protein comprises a polypeptide chain, wherein the polypeptide chain comprises VD1-(X1)n-VD2-C-(X2)n, wherein

VD1 is a first heavy chain variable domain;

VD2 is a second heavy chain variable domain;

C is a heavy chain constant domain;

X1 is a linker with the proviso that it is not CH1;

X2 is an Fc region;

(X1)n is (X1)0 or (X1)1;

(X2)n is (X2)0 or (X2)1; and

wherein the binding protein specifically binds to a disease target selected from the group consisting of Abeta, BACE, Her-2, RGMA, TNFα and APP; and

(a) VD1 or VD2 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174;

(b) VD1 and VD2 independently comprise three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174; or

(c) VD1 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174; and VD2 comprises three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174.

In certain embodiments,

(a) VD1 or VD2 comprises an amino acid sequence selected from the group consisting of SEQ ID NO: 38, 58, 93, 94, 95, 96, 97, 98, 99, 101, 167, and 169; or

(b) VD1 and VD2 independently comprise an amino acid sequence selected from the group consisting of SEQ ID NO: 38, 58, 93, 94, 95, 96, 97, 98, 99, 101, 162, and 170.

In certain embodiments, the binding protein comprises a polypeptide chain, wherein the polypeptide chain comprises VD1-(X1)n-VD2-C-(X2)n, wherein

VD1 is a first light chain variable domain;

VD2 is a second light chain variable domain;

C is a light chain constant domain;

X1 is a linker with the proviso that it is not CL;

X2 does not comprise an Fc region;

(X1)n is (X1)0 or (X1)1;

(X2)n is (X2)0 or (X2)1; and

wherein the binding protein specifically binds to a disease target selected from the group consisting of Abeta, BACE, Her-2, RGMA, TNFα, and APP; and

(a) VD1 or VD2 comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177;

(b) VD1 and VD2 independently comprise three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177; or

(c) VD1 comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177, and VD2 comprises three each comprising amino acid sequences selected CDRs from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

In certain embodiments,

(a) VD1 or VD2 comprises an amino acid sequence selected from the group consisting of SEQ ID NO: 39, 59, 87, 88, 89, 90, 91, 92, 100, 102, 163, and 171; or

(b) VD1 and VD2 independently comprise an amino acid sequence selected from the group consisting of SEQ ID NO: 39, 59, 87, 88, 89, 90, 91, 92, 100, 102, 163, and 171.

In certain embodiments of the binding protein, the (X1)n is (X1)0.

The disclosure provides a binding protein comprising first and second polypeptide chains, wherein the first polypeptide chain comprises a first VD1-(X1)n-VD2-C-(X2)n, wherein

VD1 is a first heavy chain variable domain;

VD2 is a second heavy chain variable domain;

C is a heavy chain constant domain;

X1 is a first linker;

X2 is an Fc region;

wherein the second polypeptide chain comprises a second VD1-(X1)n-VD2-C-(X2)n, wherein

VD1 is a first light chain variable domain;

VD2 is a second light chain variable domain;

C is a light chain constant domain;

X1 is a second linker;

X2 does not comprise an Fc region;

(X1)n is independently (X1)0 or (X1)1 and (X2)n is independently (X2)0 or

(X2)1

wherein the first and second X1 linker are the same or different;

wherein the first X1 linker is not CH1 and/or the second X1 linker is not CL;

wherein the binding protein specifically binds to a disease target selected from the group consisting of Abeta, BACE, Her-2, RGMA, TNFα and APP; and

(a) VD1 or VD2 heavy chain variable domain comprises three CDRs each, wherein at least one CDR comprises an amino acid sequence selected from the group consisting of SEQ ID NO: 109, 110, 111, 121, 123, 136, 137, 138, 139, 140, 141, 142, 143, 147, 148, 149, 164, 165, 166, 172, 173, and 174;

(b) VD1 and VD2 heavy chain variable domains independently comprise three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174; or

(c) VD1 heavy chain variable domain comprises three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174; and VD2 heavy chain variable domain comprises three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174;

and wherein

(a) VD1 or VD2 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152;

(b) VD1 and VD2 light chain variable domains independently comprise three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177; or

(c) VD1 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177, and VD2 light chain variable domain comprises three each comprising amino acid sequences selected CDRs from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

In certain embodiments,

(a) VD1 or VD2 heavy chain variable domain comprises an amino acid sequence selected from the group consisting of SEQ ID NO: 38, 58, 93, 94, 95, 96, 97, 98, 99, 101, 162, and 170; or

(b) VD1 and VD2 heavy chain variable domains independently comprise an amino acid sequence selected from the group consisting of SEQ ID NO: 38, 58, 93, 94, 95, 96, 97, 98, 99, 101, 162, and 170;

and wherein

(a) VD1 or VD2 light chain variable domain comprises an amino acid sequence selected from the group consisting of SEQ ID NO: 39, 59, 87, 88, 89, 90, 91, 92, 100, 102, 163, and 171; or

(b) VD1 and VD2 light chain variable domains independently comprise an amino acid sequence selected from the group consisting of SEQ ID NO: 39, 59, 87, 88, 89, 90, 91, 92, 100, 102, 163, and 171.

In certain embodiments of the binding protein, the X1 is any one of the sequences described herein. For example, the X1 comprises any one of SEQ ID NOs 1-29, 178 and 179.

In certain embodiments, the binding protein comprises two first polypeptide chains and two second polypeptide chains.

In certain embodiments, a first set of a first and a second polypeptide chain is as defined in claim 22 and a second set of a first and a second polypeptide chain is as defined in claim 36.

In certain embodiments, the Fc region is a variant sequence Fc region.

In certain embodiments, the Fc region is an Fc region from an IgG1, IgG2, IgG3, IgG4, IgA, IgM, IgE, or IgD. For example, the Fc region is derived from a mammal, for example a human.

In certain embodiments, the VD1 of the first polypeptide chain and the VD1 of the second polypeptide chain are from a different first and second parent antibody, respectively, or antigen binding portion thereof.

In certain embodiments, the VD2 of the first polypeptide chain and the VD2 of the second polypeptide chain are from a different first and second parent antibody, respectively, or antigen binding portion thereof.

In certain embodiments, the first and the second parent antibodies bind different epitopes on the antigen.

In certain embodiments, the binding protein comprises a polypeptide chain, wherein the polypeptide chain comprises VD1-(X1)n-VD2-C-(X2)n, wherein

VD1 is a first heavy chain variable domain;

VD2 is a second heavy chain variable domain;

C is a heavy chain constant domain;

X1 is a linker with the proviso that it is not CH1;

X2 is an Fc region;

(X1)n is (X1)0 or (X1)1;

(X2)n is (X2)0 or (X2)1; and

wherein the binding protein specifically binds TfR or HIR and Abeta, BACE, Her-2, RGMA, TNFα, or APP;

(a) VD1 or VD2 comprises three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;

(b) VD1 and VD2 independently comprise three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;

(c) VD1 comprises three CDRs each comprising amino acid sequences selected from the group consisting of SEQ ID NO: 76, 77, 78, 82, 83, 115-117 and 156-158, and VD2 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174; or

(d) VD2 comprises three CDRs each comprising amino acid sequences selected from SEQ ID NO: 76, 77, 78, 82, 83, 115-117 and 156-158, and VD1 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174.

In certain embodiments,

(a) VD1 or VD2 comprises an amino acid sequence selected from the group consisting of SEQ ID NO: 30, 32, 34, 36, 56, 104, 38, 58, 93, 94, 95, 96, 97, 98, 99, 101, 162, and 170; or

(b) VD1 comprises an amino acid sequence selected from the group consisting of SEQ ID NO: 30, 32, 34, 36, 56, or 104 and VD2 comprises an amino acid sequence selected from the group consisting of SEQ ID NO: 38, 58, 93, 94, 95, 96, 97, 98, 99, 101, 162, and 170.

In certain embodiments of the binding protein, the polypeptide chain comprises VD1-(X1)n-VD2-C-(X2)n, wherein

VD1 is a first light chain variable domain;

VD2 is a second light chain variable domain;

C is a light chain constant domain;

X1 is a linker with the proviso that it is not CL;

X2 does not comprise an Fc region;

(X1)n is (X1)0 or (X1)1;

(X2)n is (X2)0 or (X2)1; and

wherein the binding protein specifically binds TfR or HIR and Abeta, BACE, Her-2, or APP;

(a) VD1 or VD2 comprises three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;

(b) VD1 and VD2 independently comprise three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;

(c) VD1 comprises three CDRs each comprising amino acid sequences selected from the group consisting of SEQ ID NO: 79, 80, 81, 84, 85, 86, 118, 119, 120, 159, 160, and 161, and VD2 comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152, 167, 168, 169, 175, 176, and 177; or

(d) VD2 comprises three CDRs each comprising amino acid sequences selected from the group consisting of SEQ ID NO: 79, 80, 81, 84, 85, 86, 118, 119, 120, 159, 160, and 161; and VD1 comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

In certain embodiments,

(a) VD1 or VD2 comprises an amino acid sequence selected from the group consisting of SEQ ID NO: 31, 33, 35, 37, 57, 105, 106, 107, 108, 39, 59, 87, 88, 89, 90, 91, 92, 100, 102, 163, and 171; or

(b) VD1 comprises an amino acid sequence selected from the group consisting of SEQ ID NO: 31, 33, 35, 37, 57, 105, 106, 107, and 108 and VD2 comprises an amino acid sequence selected from the group consisting of SEQ ID NO: 39, 59, 87, 88, 89, 90, 91, 92, 100, 102, 163, and 171.

In one embodiment, (X1)n is (X1)0.

In certain embodiments, the binding protein comprising first and second polypeptide chains, wherein the first polypeptide chain comprises a first VD1-(X1)n-VD2-C-(X2)n, wherein

VD1 is a first heavy chain variable domain;

VD2 is a second heavy chain variable domain;

C is a heavy chain constant domain;

X1 is a first linker;

X2 is an Fc region;

wherein the second polypeptide chain comprises a second VD1-(X1)n-VD2-C-(X2)n, wherein

VD1 is a first light chain variable domain;

VD2 is a second light chain variable domain;

C is a light chain constant domain;

X1 is a second linker;

X2 does not comprise an Fc region;

(X1)n is independently (X1)0 or (X1)1 and (X2)n is independently (X2)0 or (X2)1,

wherein the first and second X1 linker are the same or different;

wherein the first X1 linker is not CH1 and/or the second X1 linker is not CL;

wherein the binding protein specifically binds TfR or HIR and Abeta, BACE, Her-2, RGMA, TNFα, or APP;

(a) VD1 or VD2 heavy chain variable domain comprises three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;

(b) VD1 and VD2 heavy chain variable domains independently comprise three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;

(c) VD1 heavy chain variable domain comprises three CDRs each comprising amino acid sequences selected from the group consisting of SEQ ID NO: 76, 77, 78, 82, 83, 115, 116, 117, 156, 157, 158, 164, 165, 166, 172, 173, and 174, and VD2 heavy chain variable domain comprises three CDRs each comprising amino acid sequences selected from the group consisting of SEQ ID NO: 109, 110, 111, 121, 123, 136, 137, 138, 139, 140, 141, 142, 143, 147, 148, and 149; or

(d) VD2 heavy chain variable domain comprises three CDRs each comprising amino acid sequences selected from the group consisting of SEQ ID NO: 76, 77, 78, 82, 83, 115, 116, 117, 156, 157, 158, 164, 165, 166, 172, 173, and 174, and VD1 heavy chain variable domain comprises three CDRs each comprising amino acid sequences selected from the group consisting of SEQ ID NO: 109, 110, 111, 121, 122, 123, 136, 137, 138, 139, 140, 141, 142, 143, 147, 148, and 149; and wherein

(a) VD1 or VD2 light chain variable domain comprises three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;

(b) VD1 and VD2 light chain variable domains independently comprise three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;

(c) VD1 light chain variable domain comprises three CDRs each comprising amino acid sequences selected from the group consisting of SEQ ID NO: 79, 80, 81, 84, 85, 86, 118, 119, 120, 159, 160, 161, 167, 168, 169, 175, 176, and 177; and VD2 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152; or

(d) VD2 light chain variable domain comprises three CDRs each comprising amino acid sequences selected from the group consisting of SEQ ID NO: 79, 80, 81, 84, 85, 86, 118, 119, 120, 159, 160, 161, 167, 168, 169, 175, 176, and 177; and VD1 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152.

In certain embodiments,

(a) VD1 or VD2 heavy chain variable domain comprises an amino acid sequence selected from the group consisting of SEQ ID NO: 30, 32, 34, 36, 56, 104, 38, 58, 93, 94, 95, 96, 97, 98, 99, 101, -162, and 170; or

(b) VD1 heavy chain variable domain comprises an amino acid sequence selected from the group consisting of SEQ ID NO: 30, 32, 34, 36, 56, or 104 and VD2 heavy chain variable domain comprises an amino acid sequence selected from the group consisting of SEQ ID NO: 38, 58, 93, 94, 95, 96, 97, 98, 99, 101, 162, and 170;

and wherein

(a) VD1 or VD2 light chain variable domain comprises an amino acid sequence selected from the group consisting of SEQ ID NO: 31, 33, 35, 37, 57, 105, 106, 107, 108, 39, 59, 87, 88, 89, 90, 91, 92, 100, 102, 163, and 171; or

(b) VD1 light chain variable domain comprises an amino acid sequence selected from the group consisting of SEQ ID NO: 31, 33, 35, 37, 57, 105, 106, 107, or 108 and VD2 light chain variable domain comprises an amino acid sequence selected from the group consisting of SEQ ID NO: 39, 59, 87, 88, 89, 90, 91, 92, 100, or 102, 163, and 171.

In certain embodiments, X1 comprises any one of SEQ ID NOs 1-29, 178 and 179.

In certain embodiments, the binding protein comprises two first polypeptide chains and two second polypeptide chains.

In certain embodiments, the Fc region is a variant sequence Fc region. In certain embodiments, the Fc region is an Fc region from an IgG1, IgG2, IgG3, IgG4, IgA, IgM, IgE, or IgD.

In certain embodiments, the VD1 of the first polypeptide chain and the VD1 of the second polypeptide chain are from a different first and second parent antibody, respectively, or antigen binding portion thereof.

In certain embodiments, the VD2 of the first polypeptide chain and the VD2 of the second polypeptide chain are from a different first and second parent antibody, respectively, or antigen binding portion thereof.

In certain embodiments, the first and the second parent antibodies bind different antigens.

The disclosure provides a monospecific binding protein comprising the heavy polypeptide chain of claim 17 and the light polypeptide chain of claim 19 or the heavy polypeptide claim of claim 31 and the light polypeptide chain of claim 33.

The disclosure provides a bispecific binding protein comprising the heavy polypeptide chain of claim 17 and the light polypeptide chain of claim 33; the heavy polypeptide claim of claim 31 and the light polypeptide chain of claim 19; the heavy polypeptide claim of claim 46 and the light polypeptide chain of claim 19, 33 or 48; or the heavy polypeptide claim of claim 17, 31 or 46 and the light polypeptide chain of claim 48.

In any of the embodiments herein, for example claim 1, wherein the binding protein comprises Out1-(X1)m-In1-(X2)n, wherein In1 specifically binds to the antigen expressed on the brain vascular epithelium of the subject, wherein Out1 specifically binds to another molecule, wherein X1 is a linker, wherein X2 is an Fc region, wherein m is 0 or 1 and wherein n is 0 or 1.

In certain embodiments, the In1 specifically binds to the antigen expressed on the brain vascular epithelium of the subject with an EC50 of between about 5 nM and 0.01 nM. In certain embodiments, the In1 specifically binds transferrin receptor. In certain embodiments, the In1 specifically binds the transferrin receptor with an EC50 less than 3 nM. In certain embodiments, the In1 comprises the amino sequence of SEQ ID NO:56. In certain embodiments, the X1 comprises the amino acid sequence of SEQ ID NO:179.

In certain embodiments, the Out1 binds another molecule selected from the group consisting of CGRP, TNFα, RGMA, Substance P, Bradykinin, Nav1.7, LPA, P2X3, NGF, Abeta; BACE1; IL-1β; IGF1, or 2; IL-18; IL-6; RAGE; NGF; EGFR; cMet, Her-2 and CD-20.

In certain embodiments, the Out1 specifically binds to the antigen expressed on the brain vascular epithelium of the subject with an EC50 of between about 1 nM and 100 nM. In certain embodiments, the Out1 specifically binds transferrin receptor. In certain embodiments, the Out 1 specifically binds the transferrin receptor with an EC50 greater than 3 nM. In certain embodiments, the Out1 comprises the amino sequence of SEQ ID NO:36. In certain embodiments, the X1 comprises the amino acid sequence of SEQ ID NO:21.

In certain embodiments, the In1 binds another molecule selected from the group consisting of CGRP, TNFα, RGMA, Substance P, Bradykinin, Nav1.7, LPA, P2X3, NGF, Abeta; BACE1; IL-1β; IGF1, or 2; IL-18; IL-6; RAGE; NGF; EGFR; cMet, Her-2 and CD-20.

In any of the embodiments herein, the first parent antibody or antigen binding portion thereof, in certain embodiments binds the first antigen with a potency different from the potency with which the second parent antibody or antigen binding portion thereof, binds the second antigen.

In any of the embodiments herein, the first parent antibody or antigen binding portion thereof, in certain embodiments binds the first antigen with an affinity different from the affinity with which the second parent antibody or antigen binding portion thereof, binds the second antigen.

In any of the embodiments herein, the binding protein in certain embodiments has an on rate constant (Kon) to the one or more targets of at least about 10²M⁻¹s⁻¹; at least about 10³M¹s¹; at least about 10⁴M⁻¹s⁻¹; at least about 10⁶M⁻¹s⁻¹; or at least about 10⁶M⁻¹s⁻¹, as measured by surface plasmon resonance.

In any of the embodiments herein, the binding protein in certain embodiments has an off rate constant (Koff) to the one or more targets of at most about 10⁻³s⁻¹; at most about 10⁻⁴s⁻¹; at most about 10⁻⁵s⁻¹; or at most about 10⁻⁶s⁻¹, as measured by surface plasmon resonance.

In any of the embodiments herein, the binding protein in certain embodiments has a dissociation constant (K_{d}) to the one or more targets of at most about 10⁻⁷ M; at most about 10⁻⁸ M; at most about 10⁻⁹ M; at most about 10⁻¹⁰ M; at most about 10⁻¹¹ M; at most about 10⁻¹² M; or at most 10⁻¹³M.

A further embodiment, of any of the heavy chain, light chain, two chain, or four chain embodiments, includes at least one X1 linker comprising AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9) SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); or G/S based sequences (e.g., G4S and G4S repeats; SEQ ID NO: 29). In an embodiment, X2 is an Fc region. In another embodiment, X2 is a variant Fc region. In various embodiments, the linker comprises any of the sequences described herein, for example SEQ ID NOs: 1-29, 178, or 179.

The disclosure provides a binding protein conjugate comprising a binding protein in any of the embodiments herein or in any of the claims. In certain embodiments, the binding protein conjugate further comprises an agent, wherein the agent is an immunoadhension molecule, a diagnostic agent, an imaging agent, a therapeutic agent, or a cytotoxic agent.

In certain embodiments, the imaging agent is a radiolabel, an enzyme, a fluorescent label, a luminescent label, a bioluminescent label, a magnetic label, or biotin.

In any of the embodiments herein, the binding protein is a crystallized binding protein.

An isolated nucleic acid encoding any one of the binding proteins disclosed herein is also provided. A further embodiment provides a vector comprising the isolated nucleic acid disclosed herein wherein the vector is pcDNA; pTT (Durocher et al. (2002) Nucleic Acids Res. 30(2); pTT3 (pTT with additional multiple cloning site; pEFBOS (Mizushima and Nagata (1990) Nucleic Acids Res. 18(17); pBV; pJV; pcDNA3.1 TOPO; pEF6 TOPO; pBOS; pHybE; or pBJ. In an embodiment, the vector is a vector disclosed in US Patent Publication No. 20090239259, incorporated by reference in its entirety.

The disclosure provides an isolated nucleic acid encoding the binding protein amino acid sequence in any of the embodiments described herein or any of the claims.

In certain embodiments, the disclosure provides a vector comprising an isolated nucleic acid described herein. For example, the vector comprises the isolated nucleic acid described in claim 82. In certain embodiments, the vector is pcDNA, pTT, pTT3, pEFBOS, pBV, pJV, pcDNA3.1 TOPO, pEF6 TOPO, pHybE, pBOS or pBJ.

In another aspect, a host cell is transformed with the vector disclosed herein. In an embodiment, the host cell is a prokaryotic cell, for example, E. coli. In another embodiment, the host cell is a eukaryotic cell, for example, a protist cell, an animal cell, a plant cell, or a fungal cell. In an embodiment, the host cell is a mammalian cell including, but not limited to, CHO, COS, NS0, SP2, PER.C6, or a fungal cell, such as Saccharomyces cerevisiae, or an insect cell, such as Sf9. In an embodiment, two or more binding proteins, e.g., with different specificities, are produced in a single recombinant host cell. For example, the expression of a mixture of antibodies has been called Oligoclonics™ (Merus B. V., The Netherlands) U.S. Pat. Nos. 7,262,028 and 7,429,486.

The disclosure provides also a method of producing a binding protein disclosed herein comprising culturing any one of the host cells disclosed herein in a culture medium under conditions sufficient to produce the binding protein is provided. In an embodiment, 50%-75% of the binding protein produced by this method is a dual specific tetravalent binding protein. In another embodiment, 75%-90% of the binding protein produced by this method is a dual specific tetravalent binding protein. In another embodiment, 90%-95% of the binding protein produced is a dual specific tetravalent binding protein. The disclosure provides a method for treating a mammal comprising administering to the mammal an effective amount of the binding protein.

The disclosure provides a pharmaceutical composition comprising the binding protein described herein including for example the binding protein of any of the claims, and a pharmaceutically acceptable carrier.

In certain embodiments, the composition further comprises at least one additional therapeutic agent. In certain embodiments, the additional therapeutic agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

In certain embodiments, the additional therapeutic agent is selected from the group consisting of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody or agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1 RI, sIL-1RII, sIL-6R, an anti-inflammatory cytokine, IL-4, IL-10, IL-11, IL-13, TGFβ and combinations thereof.

The disclosure provides the binding protein in any of the embodiments herein for use in treating a subject for a disease or a disorder by administering to the subject the binding protein such that treatment is achieved.

In certain embodiments, the disorder is a brain disorder. In various embodiments, the brain disorder is an autoimmune or inflammatory disease of the brain, an infectious disorder of the brain, a neurological disorder, a neurodegenerative disorder, a brain cancer, or a brain metastasis.

In certain embodiments, the disorder is selected from the group consisting of: Huntington's chorea, Parkinson's disease, Alzheimer's disease, stroke, mental disorders, depression, schizophrenia, acute pain, and chronic pain.

In certain embodiments of the method, administering to the subject is parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, or transdermal. In certain embodiments, the method further comprises observing a reduction in indicia of the disorder.

The disclosure provides a method for generating a binding protein capable of binding two antigens, the method comprising the steps of:

a) obtaining a first parent antibody or antigen binding portion thereof, capable of binding a first antigen;

b) obtaining a second parent antibody or antigen binding portion thereof, capable of binding a second antigen;

c) preparing construct(s) encoding the polypeptide chain(s) of any of the preceding claims; and

d) expressing the polypeptide chain(s); such that the binding protein capable of binding the first and the second antigen is generated.

In certain embodiments, the Fc region is a variant sequence Fc region. In certain embodiments, the Fc region is an Fc region from an IgG1, IgG2, IgG3, IgG4, IgA, IgM, IgE, or IgD.

In certain embodiments, the first parent antibody or antigen binding portion thereof, if present, binds the first antigen with a different affinity and/or potency than the affinity and/or potency with which the second parent antibody or antigen binding portion thereof, if present, binds the second antigen.

The disclosure also provides a method of determining the presence of at least one antigen or fragment thereof in a test sample by an immunoassay.

In certain embodiments, the immunoassay comprises contacting the test sample with at least one binding protein and at least one detectable label, wherein the at least one binding protein comprises the binding protein of any of the preceding claims.

In certain embodiments, the method further comprises:

(i) contacting the test sample with the at least one binding protein, wherein the binding protein binds to an epitope on the antigen or fragment thereof so as to form a first complex;

(ii) contacting the first complex with the at least one detectable label, wherein the detectable label binds to the binding protein or an epitope on the antigen or fragment thereof that is not bound by the binding protein to form a second complex; and

(iii) detecting the presence of the antigen or fragment thereof in the test sample based on the signal generated by the detectable label in the second complex, wherein the presence of the antigen or fragment thereof is identified or indicated by analyzing the signal generated by the detectable label.

In certain embodiments, the method further comprising:

(i) contacting the test sample with the at least one binding protein, wherein the binding protein binds to an epitope on the antigen or fragment thereof so as to form a first complex;

(ii) contacting the first complex with the at least one detectable label, wherein the detectable label competes with the antigen or fragment thereof for binding to the binding protein so as to form a second complex; and

(iii) detecting the presence of the antigen or fragment thereof in the test sample based on the signal generated by the detectable label in the second complex, wherein the presence of the antigen or fragment thereof is measured by analyzing the signal generated by the detectable label.

In certain embodiments, the test sample is from a patient and the method further comprises diagnosing, prognosticating, or assessing the efficiency of therapeutic/prophylactic treatment of the patient, and optionally wherein if the method further comprises assessing the efficacy of therapeutic/prophylactic treatment of the patient, the method optionally further comprises modifying the therapeutic/prophylactic treatment of the patient as needed to improve efficacy.

In certain embodiments, the method is adapted for use in an automated system or a semi-automated system. In certain embodiments, wherein the method determines the presence of more than one antigen in the sample.

The disclosure provides a method of determining the amount or concentration of an antigen or fragment thereof in a test sample by an immunoassay,

In certain embodiments, the immunoassay (a) employs at least one agent and at least one detectable label and (b) comprises comparing a signal generated by the detectable label with a control or a calibrator comprising the antigen or fragment thereof, wherein the calibrator is optionally part of a series of calibrators in which each calibrator differs from the other calibrators in the series by the concentration of the antigen or fragment thereof, wherein the at least one agent comprises the binding protein of any of the preceding claims.

In certain embodiments, the method further comprising:

(i) contacting the test sample with the at least one binding protein, wherein the binding protein binds to an epitope on the antigen or fragment thereof so as to form a first complex;

(ii) contacting the first complex with the at least one detectable label, wherein the detectable label binds to an epitope on the antigen or fragment thereof that is not bound by the binding protein to form a second complex; and

(iii) determining the amount or concentration of the antigen or fragment thereof in the test sample based on the signal generated by the detectable label in the second complex, wherein the amount or concentration of the antigen or fragment thereof is identified by analyzing the signal generated by the detectable label.

In certain embodiments, the method further comprising:

(i) contacting the test sample with the at least one binding protein, wherein the binding protein binds to an epitope on the antigen or fragment thereof so as to form a first complex;

(ii) contacting the complex with the at least one detectable label, wherein the detectable label competes with the antigen or fragment thereof for binding to the binding protein so as to form a second complex; and

(iii) determining the amount or concentration of the antigen or fragment thereof in the test sample based on the signal generated by the detectable label in the second complex, wherein the presence of the antigen or fragment thereof is indicated by analyzing the signal generated by the detectable label.

In certain embodiments, the test sample is from a patient and the method further comprises diagnosing, prognosticating, or assessing the efficiency of therapeutic/prophylactic treatment of the patient, and wherein if the method further comprises assessing the efficacy of therapeutic/prophylactic treatment of the patient, the method optionally further comprises modifying the therapeutic/prophylactic treatment of the patient as needed to improve efficacy.

In certain embodiments, the method is adapted for use in an automated system or a semi-automated system. In certain embodiments, the method determines the amount or concentration of more than one antigen in the sample.

The disclosure also provides a kit for assaying a test sample for the presence, amount, or concentration of an antigen or fragment thereof, the kit comprising

(a) instructions for assaying the test sample for the antigen or fragment thereof and

(b) at least one binding protein comprising the binding protein of any of the preceding claims.

The disclosure provides a humanized antibody that specifically binds TfR comprising an amino acid sequence selected from the group consisting of SEQ ID NO: 30-37, 56 and 57.

The disclosure provides a humanized antibody that specifically binds HIR comprising an amino acid sequence selected from the group consisting of SEQ ID NO: 104-108.

In certain embodiments further comprising a polypeptide comprising an amino acid sequence of SEQ ID NO:103, wherein the polypeptide can be bound to the binding protein or unbound to the binding protein.

### BRIEF DESCRIPTION OF THE DRAWINGS

FIG. 1 is a schematic representation of Dual Variable Domain (DVD) binding protein constructs.

FIG. 2 is a micrograph of brain tissue showing elevation of TfR mAb in brain extracts (MSD-ECL) localized to parenchyma and neuronal cell bodies (IHC) after therapeutic dosing.

FIG. 3 is a micrograph of brain tissue showing elevated DVD-Ig™ levels were localized to Purkinje cells in cerebellum and cranial nerve cell bodies by IHC.

FIG. 4 is a flow chart representation of methods and systems used for generation and in vitro/in vivo screening of receptor-mediated transcytosis domain DVD-Igs.

FIG. 5 is a drawing/representation of an exemplary DVD Ig. The DVD immunoglobulin includes at least one variable domain that specifically binds a BBB antigen (anti-BBB antigen), and a different at least one variable domain that specifically binds target X. For example, the DVD-Ig in various embodiments is a TNF/TfR, RGMA/TfR, Abeta/TfR, and Her2/TfR.

FIG. 6 is a drawing/representation of an in vivo tissue distribution protocol used for analyzing characteristics of antibody or a DVD-Ig™.

FIG. 7 is a micrograph of stained brain tissue from subjects administered either: 40 mpk of control human IgG at 24 hours; 20 mpK of a 8C11-hFc DVD at 48 hours; 30 mpK of a non-specific DVD control at 48 hours; 20 mpk of TNF-GS-AB221 DVD at 24 hours; or 20 mpk of TfR(AB405)-SL-TNF DVD at 24 hours.

FIG. 8 is a bar graph showing the percent maximum possible effect (% MPE; ordinate) at day 1 and day 5 for BALB-C murine subjects 15 days after Bennett surgery and after intrathecally injection (abscissa) with: control IgG (48 μg/10 μl dose per injection); 8C11-GS-AB221 DVD-Ig (anti-TNFa/anti-TfR; 55 μg/10 μl dose per injection); or morphine (10 μg/10 μl dose per injection). The injections were performed daily for five days after the Bennett surgery. Mechanical allodynia was assessed in the Bennett model 120 minutes post-injection administrations at day 1 and day 5.

FIG. 9 is a bar graph showing the percent efficacy (ordinate) at day 1 and day 5 for murine subjects 15 days after intravenous injection (abscissa) with control IgG (48 μg/10 μl/dose per injection); 8C11-GS-AB221 DVD (anti-TNFa/anti-TfR; 55 μg/10 μl/dose per injection); or an acute post-operation dose of gabapentin (10 μg/10 μl/dose per injection). The injections were performed daily for five days after the Bennett surgery. Mechanical allodynia was assessed in the above Bennett model 120 minutes post-injection administration at day 1 and at day 5.

FIG. 10 is a micrograph of stained brain tissue from subjects administered either: 40 mpk of RGMA (AE12-1)-hFc at 24 hours; 30 mpk human IgG control; 20 mpk of RGMA (AE12-1)-GS-AB403 DVD-Ig, or 30 mpk of RGMA (AE12-1)-GS-AB403 DVD-Ig.

### DETAILED DESCRIPTION

In certain aspects, the invention provides multivalent and/or multispecific binding proteins capable of binding receptors expressed on the brain vascular epithelium. Structures at the blood brain barrier (“BBB”) enabling such transport include but are not limited to the insulin receptor, transferrin receptor, LRP, melanocortin receptor, nicotinic acetylcholine receptor, VACM-1 receptor, vascular endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, S1P1, P2Y receptor and RAGE. In addition, strategies enable the use of binding proteins also as shuttles to transport potential drugs into the CNS including low molecular weight drugs, nanoparticles and nucleic acids (Coloma et al. (2000) Pharm Res. 17(3):266-74; Boado et al. (2007) Bioconjug. Chem. 18(2):447-55). Dual variable domain binding proteins (DVD binding proteins) or dual variable domain immunoglobulins (DVD-IG™™), and pharmaceutical compositions thereof, as well as nucleic acids, recombinant expression vectors and host cells for making such DVD binding proteins are also provided. Methods of using the DVD binding proteins to detect specific antigens, either in vitro or in vivo are also provided.

In certain embodiments, multivalent and/or multispecific binding proteins bind to the binding receptors expressed on the brain vascular epithelium as well as a therapeutic target. These therapeutic targets include for example CGRP, TNFα, RGMA, Substance P, Bradykinin, Nav1.7, LPA, P2X3, NGF, Abeta; BACE1; IL-1β; IGF1, or 2; IL-18; IL-6; RAGE; NGF; EGFR; cMet; Her-2; and CD-20. The binding protein or peptide in various embodiments comprises an amino acid sequence that specifically binds to an epitope, antigen, receptor or target, such that the binding protein or peptide is effective for transport to or across the BBB. For example, the amino acid sequence includes at least about three amino acids, at least about five amino acids, at least about seven amino acids, at least about ten amino acids, at least about 15 amino acids, or at least 20 amino acids that binds to an epitope, antigen, receptor or target, such that the binding protein or peptide is effective for transport to or across the BBB. In these embodiments, the epitope, antigen, receptor or target includes for example an insulin receptor, a transferrin receptor, a low density lipoprotein receptor-related protein (LRP) for example LRP-1 and LRP-8, melanocortin receptor, nicotinic acetylcholine receptor, VACM-1 receptor, vascular endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, S1P1, P2Y receptor, and RAGE.

In other embodiments, the binding protein or peptide is also capable of modulating a biological function of one or more targets. In certain aspects of this embodiment, the binding protein or peptide comprises an amino acid sequence that specifically binds to an epitope, antigen, receptor or target, such that a biological function is modulated. In these embodiments, the epitope, antigen, receptor or target can be selected from CGRP, TNFα, RGMA, Substance P, Bradykinin, Nav1.7, LPA, P2X3, NGF, Abeta; BACE1; IL-1β; IGF1, or 2; IL-18; IL-6; RAGE; NGF; EGFR; cMet; Her-2; and CD-20.

In various embodiments, the binding protein or peptide comprises; a Fv; a Fab; a Fab′; a F(ab′)₂, or a bivalent fragment comprising two Fab fragments linked by a disulfide bridge at the hinge region. In other embodiments, the binding protein includes at least one heavy variable region (VH) and at least one light variable region (VL). In certain embodiments the binding protein or peptide includes a VH and CH1 domains; VL and VH domains; or an isolated complementarity determining region (CDR). In various embodiments, the binding protein or peptide comprises at least one VH, at least one VL, or at least one hypervariable (hv) site. In various embodiments, the binding or peptide includes a constant region. For example, the constant region is from a mammal, e.g., a human and a mouse.

In certain embodiments, the binding protein is monospecific for an epitope or antigen. In these embodiments, the binding protein or peptide is effective for transport to or across the BBB. In other embodiments, two or more distinct binding regions are combined to construct a chimeric binding protein or peptide. For example the chimeric protein includes at least two non-identical binding regions. For example, the binding protein comprises a DVD-Ig™ as described herein.

The binding protein or peptide includes at least one binding region that specifically binds an epitope, an antigen, a receptor or a target. In various embodiments, the binding protein comprises a single chain. In various embodiments, the binding protein comprises a plurality of chains, i.e., at least two polypeptide chains. In various embodiments, the binding protein or peptide comprises a plurality of binding regions which are ordered or orientated such that each binds to the same or different portion of an epitope, an antigen, a receptor or a target. For example, the at least one binding region is positioned proximally and/or distally to another binding region, such that each is present on the same or different variable region/domain. In various embodiments, the binding regions are positioned parallel to one another, for example on a VH and a VL. In various embodiments, the binding regions are positioned opposite or facing one another, for example a first binding region is within a first VH or first VL, and a second binding region is within a second VH or a second VL. In various embodiments, the multiple binding regions are each bound to the same separate/third portion (e.g., a constant domain or linker), such that each binding region may interact or alternatively does not interact with one another.

In various embodiments, the binding protein is a molecule with the ability to monospecifically bind a receptor, antigen or target and cross the BBB. In various embodiments, the binding protein is formulated, compounded or administered in a form (e.g., nanoparticle; liposome, mixture, or solution) and is delivered along with an agent to the brain. For example, the binding protein is administered in a composition including the agent (e.g., a peptide or protein). In various embodiments, the binding protein in bound or attached to the agent. For example, the binding protein and agent are administered within the composition. Alternatively, the agent or binding protein is administered before or after one another over a period of seconds, minutes, hours or days of one another.

In various embodiments, the binding protein is bi-specific and binds two different antigens (or epitopes). For example, the binding protein specifically binds a receptor, antigen or target for crossing the BBB, and also specifically binds another target in the brain. In various embodiments, the binding protein comprises at least one VH and at least one VL. For example, the binding protein comprises a DVD-Ig™ as described herein.

In certain embodiments, the binding protein includes at least two VH domains. In some embodiments, one VH domain specifically binds a receptor, antigen or target for crossing the BBB and another VH domain specifically binds another target in the brain. In other embodiments, the binding protein includes at least two VL domains. In some embodiments, one VL domain specifically binds a receptor, antigen or target for crossing the BBB and another VL domain specifically binds another target in the brain. In other embodiments, the binding protein includes at least two VH and at least two VL domains. In some embodiments, one VL domain specifically binds a receptor, antigen or target for crossing the BBB and another VH domain specifically binds another target in the brain while one VH domain specifically binds a receptor, antigen or target for crossing the BBB and another VH domain specifically binds another target in the brain.

According to other embodiments, the binding protein is made up of two polypeptides or arms. Each of the arms can have one or more VH and VL domains. In certain embodiments, each arm has two VH and two VL domains. In other embodiments, the arms have only two VH or two VL domains. In certain embodiments, the binding protein comprises two arms/regions and each arm binds the same target or binds at least two different targets. For example, one arm binds the receptor, antigen or target for crossing the BBB, and the other arm upon crossing the BBB binds a different target on or in the brain (brain target). For example, a VH or VL on one arm binds the receptor for crossing the BBB, and a VH or VL on the other arm binds to the target. Alternatively, in various embodiments the binding protein has two identical antigen binding arms, in which each arm contains a VH/VL that binds the receptor for crossing the BBB, and a VH/VL that binds to a target found inside of the brain upon crossing the BBB. For example, each arm has identical specificity and identical CDR sequences. In various embodiments, the binding protein is a DVD-Ig that contains a VH1 or VH2 that binds to the BBB receptor or binds to the target on or in the brain. For example, the VH1 or VL1 binds to the BBB receptor, and the VH2 or VL2 binds to the target on or in the brain. Alternatively, the VH2 or VL2 binds to the BBB receptor, and the VH1 or VL2 binds to the target on or in the brain.

In various embodiments, the binding protein comprises a DVD-Ig™ as described herein that binds at least two different targets. In various embodiments, the binding protein is a DVD-Ig contains a VH1 that binds to the BBB receptor, and a VH2 that binds to the target on or in the brain. Alternatively, the binding protein is a DVD-Ig contains a VH2 that binds to the BBB receptor, and a VH1 that binds to the target on or in the brain. In various embodiments, the VL1 binds to the BBB receptor, and the VL2 binds to the target on or in the brain. In various embodiments, the VL2 binds to the BBB receptor, and the VL1 binds to the target on or in the brain.

In various embodiments, the binding protein or peptide comprises a variable binding region. For example, the variable binding region comprises a VH or VL. In various embodiments, the VL is located proximally or distally to the VL. For example, the VH is adjacent, bound or connected to the VL. In various embodiments, the VH is directly contacted to the VL, or the VH is connected to the VL by a linker. In various embodiments, the VH is parallel or adjacent to the VL. For example, the VH is separated from the VL by a covalent bond that maintains the VH and the VL in a confirmation or orientation.

In various embodiments, the binding protein or peptide comprises a polypeptide chain having a structure VD1-(X1)n-VD2-C-(X2)n, such that VD1 is a first variable domain, VD2 is a second variable domain, C is a constant domain, X1 represents an amino acid or polypeptide, X2 represents an Fc region and n is 0 or 1. In an embodiment, the VD1 and VD2 in the binding protein are heavy chain variable domains. In another embodiment, VD1 and VD2 are capable of binding the same antigen. In another embodiment, VD1 and VD2 are capable of binding different antigens. In still another embodiment, C is a heavy chain constant domain. For example, X1 is a linker with the proviso that X1 is not CH1.

In an embodiment, the binding protein or peptide disclosed herein comprises a polypeptide chain that binds the epitope, receptor or antigen, such that the polypeptide chain comprises VD1-(X1)n-VD2-C-(X2)n, and VD1 is a first heavy chain variable domain, VD2 is a second heavy chain variable domain, C is a heavy chain constant domain, X1 is a linker, and X2 is an Fc region. In an embodiment, X1 is a linker with the proviso that it is not CH1.

In various embodiments, the binding protein or peptide is attached or linked to an agent (e.g., a therapeutic agent or diagnostic agent). For example the binding protein or peptide includes a linker that separates the binding regions and/or that separates the binding protein or peptide from the agent. The linker in a related embodiment separates the binding regions and/or subunits of the binding protein or peptide. In certain embodiments, the binding protein or peptide includes a linker that covalently joins at least one binding region (e.g., a VH or a VL) to at least one other amino acid residue or domain. In various embodiments, the linker includes at least one selected from the group of a peptide, a protein, a sugar, or a nucleic acid. In a related embodiment, the linker includes an amino acid sequence described herein or a portion thereof or multiples thereof. The linker in various embodiments stabilizes the binding protein or peptide and does not prevent the respective binding of a binding region or the peptide to the epitope, antigen, receptor or target, such that the protein or peptide is effective for transport to or across the BBB. In various embodiments, the binding protein comprises a linker that reduces steric hindrance.

In various embodiments, the binding protein peptide is recombinantly produced. In certain embodiments, the recombinant binding protein is encoded by a nucleotide sequence or the binding protein includes an amino acid sequence that is substantially identical or homologous to the sequences described herein, for example a sequence shown in any of the Examples and Tables herein. For example, recombinant binding protein or peptide is engineered and constructed using any of the sequences described herein. In a related embodiment, the binding protein or peptide is administered to a subject using a vector carrying a nucleotide sequence that encodes the binding protein or peptide. In various embodiments, the binding protein or peptide (with or without an agent) is delivered for example using a liposome, a lipid/polycation (LPD), a peptide, a nanoparticle, a gold particle, and a polymer.

In various embodiments, the binding protein or peptide includes an amino acid sequence having a conservative sequence modification from the sequences shown herein, e.g., SEQ ID NOs: 1-185 or sequences in Tables 1-18. The phrase “conservative sequence modifications” refers to amino acid modifications that do not significantly affect or alter the characteristics (e.g., binding, stability, and orientation) of the binding protein, e.g., amino acid sequences of binding protein that present a side chain at the same relative position to allow for function in a manner similar to an unmodified binding protein. A conservative modification includes for example a substitution, addition, or deletion in the amino acid sequence of the binding protein or peptide. Modification of the amino acid sequence of recombinant multimeric binding protein is achieved using any known technique in the art e.g., site-directed mutagenesis or PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. Conservative amino acid substitutions are modifications in which the amino acid residue is replaced with an amino acid residue having a similar side chain such as replacing a small amino acid with a different small amino acid, a hydrophilic amino acid with a different hydrophilic amino acid, etc.

In some embodiments, the multivalent binding protein has a molecular weight of greater than 150 kilodaltons (kD). In other embodiments, the binding protein has a molecular weight between 150 kD and 1000 kD. In other embodiments, the binding protein has a molecular weight between 150 kD and 500 kD, 150 kD and 350 kD, 150 kD and 250 kD and 150 kD and 750 kD. In other embodiments, the binding protein has a molecular weight of greater than 150, 200, 250, 300, 450, 500, 550, 600, 650, 700, 750, 800, 850, 900, 950, 1000, 1050, 1100, 1150, 1200, 1250, 1300, 1350, 1400, 1450 and 1500 kD.

In certain embodiments, the DVD binding proteins can bind to an antigen (e.g., a target and a receptor) expressed on the brain vascular epithelium and have another unoccupied binding site. This unoccupied binding site can be specific for a composition (e.g., an endogenous or exogenous therapeutic protein) to be co-transported across the BBB. Accordingly, binding proteins “pre-loaded” in this fashion can be delivered to a desired target site in the brain to exert its desired therapeutic activity. Alternatively, the binding site can remain unoccupied following transport and BBB uptake via binding to the receptor expressed on the brain vascular epithelium so that it is capable of binding a desired target molecule on the brain side of the BBB.

In certain aspects of the disclosure, there is an inverse correlation between the binding affinity of a binding protein to an antigen (e.g., a receptor) expressed on or in the brain vascular epithelium. The binding proteins specifically bind to the receptor expressed on the brain vascular epithelium, but they can bind at the lower end of the binding affinity range for specific binding. Thus, in some embodiments, the binding protein will bind to a receptor expressed on the brain vascular epithelium with dissociation constant of between 1×10⁻⁶ M and 1×10⁻⁷. In other embodiments, the dissociation constant is between 1×10⁻⁶ M and 1×10⁻⁸. In some embodiments, lower affinity is achieved through the humanization of antibodies from non-human mammals.

In other embodiments, various portions of the binding protein will bind the receptor expressed on the brain vascular epithelium with different affinities. In certain embodiments, the binding protein is a DVD binding protein. In certain embodiments, the DVD binding protein comprises two arms. Each arm includes a heavy and a light chain. Each heavy and light chain includes a variable domain. Thus, DVD binding proteins can include 8 variable domains or 4 binding sites comprising 4 VH/VL pairs. Each of these domains can specifically bind a given antigen with a different dissociation constant. In some embodiments, the domain will bind to an antigen with dissociation constant of between 1×10⁻⁶ M and 1×10⁻⁷. In other embodiments, the dissociation constant is between 1×10⁻⁶ M and 1×10⁻⁸. In certain specific embodiments, the antigen is a receptor expressed on the brain vascular epithelium, a composition to be co-transported across the BBB or a target on the brain side of the BBB.

In certain embodiments of this disclosure, the binding protein that specifically binds to a receptor expressed on the brain vascular epithelium can have a 2 or more fold increase in uptake of a composition across the blood brain barrier (BBB) compared to a control non-specific binding protein. In other embodiments, the binding protein that specifically binds to a receptor expressed on the brain vascular epithelium can have a 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 fold or more fold increase in uptake of a composition across the BBB compared to a control non-specific binding protein.

According to certain embodiments, a composition is co-administered with the binding protein that specifically binds to a receptor expressed on the brain vascular epithelium. This composition can be directly bound to the binding protein or it can be co-administered in an unconjugated form. In embodiments, wherein the composition is bound to the binding protein, in certain embodiments, the composition is bound through a linker. The linker can be a polypeptide linker. The linker can also be a non-polypeptide linker. Many such linkers are known in the art.

In certain embodiments, the composition co-administered with the binding protein can be selected from one or more of the following budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody or agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1 RI, sIL-1RII, sIL-6R, an anti-inflammatory cytokine, IL-4, IL-10, IL-11, IL-13, and TGFβ.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Generally, nomenclatures used in connection with cell and tissue culture, molecular biology, immunology, microbiology, genetics and protein and nucleic acid chemistry and hybridization described herein are those well known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

That the disclosure may be more readily understood, select terms are defined below.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

The term “bispecific antibody” refers to an antibody that binds one antigen (or epitope) on one of its two binding arms (one pair of HC/LC), and binds a different antigen (or epitope) on its second binding arm (a different pair of HC/LC). A bispecific antibody has two distinct antigen binding arms (in both specificity and CDR sequences), and is monovalent for each antigen to which it binds. Bispecific antibodies include those generated by quadroma technology (Milstein and Cuello (1983) Nature 305(5934): 537-40), by chemical conjugation of two different monoclonal antibodies (Staerz et al. (1985) Nature 314(6012): 628-31), or by knob-into-hole or similar approaches which introduces mutations in the Fc region (Holliger et al. (1993) Proc. Natl. Acad. Sci. USA 90(14): 6444-6448).

An “affinity matured” antibody is an antibody with one or more alterations in one or more CDRs thereof which result an improvement in the affinity of the antibody for antigen, compared to a parent antibody which does not possess those alteration(s). Exemplary affinity matured antibodies will have nanomolar or even picomolar affinities for the target antigen. Affinity matured antibodies are produced by procedures known in the art. Marks et al. (1992) BioTechnology 10:779-783 describes affinity maturation by VH and VL domain shuffling. Random mutagenesis of CDR and/or framework residues is described by Barbas et al. (1994) Proc. Nat. Acad. Sci. USA 91:3809-3813; Schier et al. (1995) Gene 169:147-155; Yelton et al. (1995) J. Immunol. 155:1994-2004; Jackson et al. (1995) J. Immunol. 154(7):3310-9; Hawkins et al. (1992) J. Mol. Biol. 226:889-896 and mutation at selective mutagenesis positions, contact or hypermutation positions with an activity enhancing amino acid residue as described in U.S. Pat. No. 6,914,128.

The term “CDR-grafted antibody” refers to an antibody that comprises heavy and light chain variable region sequences in which the sequences of one or more of the CDR regions of VH and/or VL are replaced with CDR sequences of another antibody. For example, the two antibodies can be from different species, such as antibodies having murine heavy and light chain variable regions in which one or more of the murine CDRs has been replaced with human CDR sequences.

The term “humanized antibody” refers to an antibody from a non-human species that has been altered to be more “human-like”, i.e., more similar to human germline sequences. One type of humanized antibody is a CDR-grafted antibody, in which non-human CDR sequences are introduced into human VH and VL sequences to replace the corresponding human CDR sequences. A “humanized antibody” is also an antibody or a variant, derivative, analog or fragment thereof that comprises framework region (FR) sequences having substantially (e.g., at least 80%, at least 85%, at least 90%, at least 95%, at least 98% or at least 99% identity to) the amino acid sequence of a human antibody and at least one CDR having substantially the amino acid sequence of a non-human antibody. A humanized antibody may comprise substantially all of at least one, and typically two, variable domains (Fab, Fab′, F(ab′) 2, FabC, Fv) in which the sequence of all or substantially all of the CDR regions correspond to those of a non-human immunoglobulin (i.e., donor antibody) and the sequence of all or substantially all of the FR regions are those of a human immunoglobulin. The humanized antibody also may include the CH1, hinge, CH2, CH3, and CH4 regions of the heavy chain. In an embodiment, a humanized antibody also comprises at least a portion of a human immunoglobulin Fc region. In some embodiments, a humanized antibody only contains a humanized light chain. In some embodiments, a humanized antibody only contains a humanized heavy chain. In some embodiments, a humanized antibody only contains a humanized variable domain of a light chain and/or humanized variable domain of a heavy chain. In some embodiments, a humanized antibody contains a light chain as well as at least the variable domain of a heavy chain. In some embodiments, a humanized antibody contains a heavy chain as well as at least the variable domain of a light chain.

The terms “dual variable domain binding protein” and “dual variable domain immunoglobulin” refer to a binding protein that has two variable domains in each polypeptide chain of its binding arm(s) (e.g., a pair of HC/LC) (see PCT Publication No. WO 02/02773), each of which is able to bind to an antigen. In an embodiment, each variable domain binds different antigens or epitopes. In another embodiment, each variable domain binds the same antigen or epitope. In another embodiment, a dual variable domain binding protein has two identical antigen binding arms, with identical specificity and identical CDR sequences, and is bivalent for each antigen to which it binds. In an embodiment, the DVD binding proteins may be monospecific, i.e., capable of binding one antigen or multispecific, i.e., capable of binding two or more antigens. DVD binding proteins comprising two heavy chain DVD polypeptides and two light chain DVD polypeptides are referred to as a DVD-Ig™. In certain embodiments the DVD binding protein includes at least one region that binds a BBB antigen. In other embodiments the DVD-Ig further includes at least one other region that binds to or a receptor, antigen, target, cell or tissue of the brain. In an embodiment, each half of a four chain DVD binding protein comprises a heavy chain DVD polypeptide, and a light chain DVD polypeptide, and two antigen binding sites. In an embodiment, each binding site comprises a heavy chain variable domain and a light chain variable domain with a total of 6 CDRs involved in antigen binding per antigen binding site.

The terms “single chain dual variable domain immunoglobulin” or “scDVD-Ig™” or scFvDVDIg™” refer to the antigen binding fragment of a DVD molecule that is analogous to an antibody single chain Fv fragment. scDVD-Ig™ are described in U.S. Ser. No. 61/746,659, incorporated herein by reference in its entirety. scDVD-Ig™ are generally of the formula VH1-(X1)n-VH2-X2-VL1-(X3)n-VL2, where VH1 is a first antibody heavy chain variable domain, X1 is a linker with the proviso that it is not a constant domain, VH2 is a second antibody heavy chain variable domain, X2 is a linker, VL1 is a first antibody light chain variable domain, X3 is a linker with the proviso that it is not a constant domain, VL2 is a second antibody light chain variable domain, and n is 0 or 1, where the VH1 and VL1, and the VH2 and VL2 respectively combine to form two functional antigen binding sites.

The terms “DVD-Fab” or fDVD-Ig™” refer to the antigen binding fragment of a DVD-Ig™ molecule that is analogous to an antibody Fab fragment. fDVD-Ig™ are described in U.S. Ser. No. 61/746,663, incorporated herein by reference in its entirety. In certain embodiments, fDVD-Ig™ include a first polypeptide chain having the general formula VH1-(X1)n-VH2-C-(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library. In certain embodiments, the fDVD-Ig™ also include a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites. In certain embodiments, the first and second polypeptide chains combine to form a fDVD-Ig™.

The terms “receptor DVD-Ig™” constructs, or “rDVD-Ig™” refer to DVD-Ig™ constructs comprising at least one receptor-like binding domain. rDVD-Ig™ are described in U.S. Ser. No. 61/746,616, incorporated herein by reference in its entirety. Variable domains of the rDVD-Ig™ molecule may include one immunoglobulin variable domain and one non-immunoglobulin variable domain such as a ligand binding domain of a receptor, or an active domain of an enzyme. rDVD-Ig™ molecules may also comprise two or more non-Ig domains (see PCT Publication No. WO 02/02773). In rDVD-Ig™ at least one of the variable domains comprises a ligand binding domain of a receptor (RD).

The term “receptor domain” (RD), or receptor binding domain, as is generally understood by one of skill in the art, refers to the portion of a cell surface receptor, cytoplasmic receptor, nuclear receptor, or soluble receptor that functions to bind one or more receptor ligands or signaling molecules (e.g., toxins, hormones, neurotransmitters, cytokines, growth factors, or cell recognition molecules).

The terms multi-specific and multivalent IgG-like molecules or “pDVD-Ig™” are capable of binding two or more proteins (e.g., antigens). pDVD-Ig™ are described in U.S. Ser. No. 61/746,617, incorporated herein by reference in its entirety. In certain embodiments, pDVD-Ig™ are disclosed which are generated by specifically modifying and adapting several concepts. These concepts include but are not limited to: (1) forming Fc heterodimer using CH3 “knobs-into-holes” design, (2) reducing light chain missing pairing by using CH1/CL cross-over, and (3) pairing two separate half IgG molecules at protein production stage using “reduction then oxidation” approach.

In certain embodiments, the binding protein of the invention is a “half-DVD-Ig”™ derived from a DVD-Ig™. The half-DVD-Ig™ preferably does not promote cross-linking observed with naturally occurring antibodies which can result in antigen clustering and undesirable activities. See U.S. patent publication number 20120201746 published Aug. 9, 2012, and international publication number WO/2012/088302 published Jun. 28, 2012, each of which is incorporated by reference herein in its entirety.

In one embodiment, a pDVD-Ig™ construct may be created by combining two halves of different DVD-Ig™ molecules, or a half DVD-Ig™ and half IgG molecule. A pDVD-Ig™ construct may be expressed from four unique constructs to create a monovalent, multi-specific molecules through the use of heavy chain CH3 knobs-into-holes design. In another embodiment, a pDVD-Ig™ construct may contain two distinct light chains, and may utilize structural modifications on the Fc of one arm to ensure the proper pairing of the light chains with their respective heavy chains. In one aspect, the heavy chain constant region CH1 may be swapped with a light chain constant region hCk on one Fab. In another aspect, an entire light chain variable region, plus hCk, may be swapped with a heavy chain variable region, plus CH1. pDVD-Ig™ construct vectors that accommodate these unique structural requirements are also disclosed.

In some embodiments, pDVD-Ig™ contain four polypeptide chains, namely, first, second, third and fourth polypeptide chains. In one aspect, the first polypeptide chain may contain VD1-(X1)n-VD2-CH-(X2)n, wherein VD1 is a first heavy chain variable domain, VD2 is a second heavy chain variable domain, CH is a heavy chain constant domain, X1 is a linker with the proviso that it is not a constant domain, and X2 is an Fc region. In another aspect, the second polypeptide chain may contain VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region. In another aspect, the third polypeptide chain may contain VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region. In another aspect, the fourth polypeptide chain may contain VD3-(X3)n-VD4-CH-(X4)n, wherein VD3 is a third light chain variable domain, VD4 is a fourth light chain variable domain, CH is a heavy chain constant domain, X3 is a linker with the proviso that it is not a constant domain, and X4 does not comprise an Fc region. In another aspect, n is 0 or 1, and the VD1 domains on the first and second polypeptide chains form one functional binding site for antigen A, the VD2 domains on the first and second polypeptide chains form one functional binding site for antigen B, the VD3 domains on the third and fourth polypeptide chains form one functional binding site for antigen C, and the VD4 domains on the third and fourth polypeptide chains form one functional binding site for antigen D. In one embodiment, antigens A, B, C and D may be the same antigen, or they may each be a different antigen. In another embodiment, antigens A and B are the same antigen, and antigens C and D are the same antigen.

As used herein “monobody DVD-Ig™” or “mDVD-Ig™” refers to a class of binding molecules wherein one binding arm has been rendered non-functional. mDVD-Ig™ are described in U.S. Ser. No. 61/746,615, incorporated herein by reference in its entirety. In one aspect, mDVD-Ig™ possesses only one functional arm capable of binding a ligand. In another aspect, the one functional arm may have one or more binding domains for binding to different ligands. The ligand may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and combinations thereof.

In one embodiment, mDVD-Ig™ contains four polypeptide chains, wherein two of the four polypeptide chains comprise VDH-(X1)n-C-(X2)n. In one aspect, VDH is a heavy chain variable domain, X1 is a linker with the proviso that it is not CH1, C is a heavy chain constant domain, X2 is an Fc region, and n is 0 or 1. The other two of the four polypeptide chains comprise VDL-(X3)n-C-(X4)n, wherein VDL is a light chain variable domain, X3 is a linker with the proviso that it is not CH1, C is a light chain constant domain, X4 does not comprise an Fc region, and n is 0 or 1. In another aspect, at least one of the four polypeptide chains comprises a mutation located in the variable domain, wherein the mutation inhibits the targeted binding between the specific antigen and the mutant binding domain.

The Fc regions of the two polypeptide chains that have a formula of VDH-(X1)n-C-(X2)n may each contain a mutation, wherein the mutations on the two Fc regions enhance heterodimerization of the two polypeptide chains. In one aspect, knobs-into-holes mutations may be introduced into these Fc regions to achieve heterodimerization of the Fc regions. See Atwell et al. J. Mol. Biol. 1997, 270: 26-35.

As used herein “cross-over DVD-Ig™” or “coDVD-Ig™” refers to a DVD-Ig™ wherein the cross-over of variable domains is used to resolve the issue of affinity loss in the inner antigen-binding domains of some DVD-Ig™ molecules. coDVD-Ig™ are described in U.S. Ser. No. 61/746,619, incorporated herein by reference in its entirety. In certain specific embodiments, cross-over dual-variable-domain (DVD) Igs are generated by crossing over light chain and the heavy chain variable domains of a dual-variable-domain (DVD) Ig or Ig like protein. In another aspect, the length and sequence of the linkers linking the variable domains may be optimized for each format and antibody sequence/structure (frameworks) to achieve desirable properties. The disclosed concept and methodology may also be extended to Ig or Ig like proteins having more than two antigen binding domains.

The term “antiidiotypic antibody” refers to an antibody raised against the amino acid sequence of the antigen combining site of another antibody. Antiidiotypic antibodies may be administered to enhance an immune response against an antigen.

The term “biological activity” refers to any one or more biological properties of a molecule (whether present naturally as found in vivo, or provided or enabled by recombinant means). Biological properties include, but are not limited to, binding a receptor, inducing cell proliferation, inhibiting cell growth, inducing other cytokines, inducing apoptosis, and enzymatic activity.

The term “neutralizing” refers to counteracting the biological activity of an antigen when a binding protein specifically binds to the antigen. In an embodiment, the neutralizing binding protein binds to an antigen (e.g., a cytokine) and reduces biologically activity of the antigen by at least about 20%, 40%, 60%, 80%, 85% or more.

“Specificity” refers to the ability of a binding protein to selectively bind an antigen.

The term “specifically binds,” means that a binding protein or fragment thereof forms a complex with an antigen that is relatively stable under physiologic conditions. Specific binding can be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. Methods for determining whether two molecules specifically bind are well known in the art and include, for example, equilibrium dialysis, surface plasmon resonance, and the like.

The term “mammal” refers to any species that is a member of the class mammalia, including rodents, primates, dogs, cats, camelids and ungulates. The term “rodent” refers to any species that is a member of the order rodentia including mice, rats, hamsters, gerbils and rabbits. The term “primate” refers to any species that is a member of the order primates, including monkeys, apes and humans. The term “camelids” refers to any species that is a member of the family camelidae including camels and llamas. The term “ungulates” refers to any species that is a member of the superorder ungulata including cattle, horses and camelids.

“Affinity” is the strength of the interaction between a binding protein and an antigen, and is determined by the sequence of the CDRs of the binding protein as well as by the nature of the antigen, such as its size, shape, and/or charge. Binding proteins may be selected for affinities that provide desired therapeutic end-points while minimizing negative side-effects. Affinity may be measured using methods known to one skilled in the art (US 20090311253).

The term “potency” refers to the ability of a binding protein to achieve a desired effect, and is a measurement of its therapeutic efficacy. Potency may be assessed using methods known to one skilled in the art (US 20090311253).

The term “cross-reactivity” refers to the ability of a binding protein to bind a target other than that against which it was raised. Generally, a binding protein will bind its target tissue(s)/antigen(s) with an appropriately high affinity, but will display an appropriately low affinity for non-target normal tissues. Individual binding proteins are generally selected to meet two criteria. (1) Tissue staining appropriate for the known expression of the antibody target. (2) Similar staining pattern between human and tox species (mouse and cynomolgus monkey) tissues from the same organ. These and other methods of assessing cross-reactivity are known to one skilled in the art (US 20090311253).

The term “biological function” refers the specific in vitro or in vivo actions of a binding protein. Binding proteins may target several classes of antigens and achieve desired therapeutic outcomes through multiple mechanisms of action. Binding proteins may target soluble proteins, cell surface antigens, as well as extracellular protein deposits. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve distinct functions in a single binding protein molecule. The in vitro assays and in vivo models used to assess biological function are known to one skilled in the art (US 20090311253).

A “stable” binding protein is one in which the binding protein essentially retains its physical stability, chemical stability and/or biological activity upon storage. A multivalent binding protein that is stable in vitro at various temperatures for an extended period of time is desirable. Methods of stabilizing binding proteins and assessing their stability at various temperatures are known to one skilled in the art (US 20090311253).

The term “solubility” refers to the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art will be able to detect an increase or decrease in solubility of a binding protein using routine HPLC techniques and methods known to one skilled in the art (US 20090311253).

Binding proteins may be produced using a variety of host cells or may be produced in vitro, and the relative yield per effort determines the “production efficiency.” Factors influencing production efficiency include, but are not limited to, host cell type (prokaryotic or eukaryotic), choice of expression vector, choice of nucleotide sequence, and methods employed. The materials and methods used in binding protein production, as well as the measurement of production efficiency, are known to one skilled in the art (US 20090311253).

The term “immunogenicity” means the ability of a substance to induce an immune response. Administration of a therapeutic binding protein may result in a certain incidence of an immune response. Potential elements that might induce immunogenicity in a multivalent format may be analyzed during selection of the parental antibodies, and steps to reduce such risk can be taken to optimize the parental antibodies prior to incorporating their sequences into a multivalent binding protein format. Methods of reducing the immunogenicity of antibodies and binding proteins are known to one skilled in the art (US 20090311253).

The terms “label” and “detectable label” mean a moiety attached to a member of a specific binding pair, such as an antibody or its analyte to render a reaction (e.g., binding) between the members of the specific binding pair, detectable. The labeled member of the specific binding pair is referred to as “detectably labeled.” Thus, the term “labeled binding protein” refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “conjugate” refers to a binding protein, such as an antibody, that is chemically linked to a second chemical moiety, such as a therapeutic or cytotoxic agent. The term “agent” includes a chemical compound, a mixture of chemical compounds, a biological macromolecule, or an extract made from biological materials. In an embodiment, the therapeutic agents or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The terms “crystal” and “crystallized” refer to a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, CRYSTALLIZATION OF NUCLEIC ACIDS AND PROTEINS, A PRACTICAL APPROACH, 2nd ea., pp. 20 1-16, Oxford University Press, New York, N.Y., (1999).

The term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. A group of pHybE vectors (U.S. Patent Application Ser. No. 61/021,282) were used for parental antibody and DVD-binding protein cloning. V1, derived from pJP183; pHybE-hCg1,z,non-a V2, was used for cloning of antibody and DVD heavy chains with a wildtype constant region. V2, derived from pJP191; pHybE-hCk V3, was used for cloning of antibody and DVD light chains with a kappa constant region. V3, derived from pJP192; pHybE-hCl V2, was used for cloning of antibody and DVDs light chains with a lambda constant region. V4, built with a lambda signal peptide and a kappa constant region, was used for cloning of DVD light chains with a lambda-kappa hybrid V domain. V5, built with a kappa signal peptide and a lambda constant region, was used for cloning of DVD light chains with a kappa-lambda hybrid V domain. V7, derived from pJP183; pHybE-hCg1,z,non-a V2, was used for cloning of antibody and DVD heavy chains with a (234,235 AA) mutant constant region.

The terms “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. Coli; mammalian cell lines CHO, HEK 293, COS, NS0, SP2 and PER.C6; the insect cell line Sf9; and the fungal cell Saccharomyces cerevisiae.

The term “transfection” encompasses a variety of techniques commonly used for the introduction of exogenous nucleic acid (e.g., DNA) into a host cell, e.g., electroporation, calcium-phosphate precipitation, DEAE-dextran transfection and the like.

The term “cytokine” refers to a protein released by one cell population that acts on another cell population as an intercellular mediator. The term “cytokine” includes proteins from natural sources or from recombinant cell culture and biologically active equivalents of the native sequence cytokines.

The term “biological sample” means a quantity of a substance from a living thing or formerly living thing. Such substances include, but are not limited to, blood, (e.g., whole blood), plasma, serum, urine, amniotic fluid, synovial fluid, endothelial cells, leukocytes, monocytes, other cells, organs, tissues, bone marrow, lymph nodes and spleen.

The term “component” refers to an element of a composition. In relation to a diagnostic kit, for example, a component may be a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample. Thus, a “component” can include a polypeptide or other analyte as above, that is immobilized on a solid support, such as by binding to an anti-analyte (e.g., anti-polypeptide) antibody. Some components can be in solution or lyophilized for reconstitution for use in an assay.

“Control” refers to a composition known to not analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) may be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., polypeptide of interest) may entail release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

The term “specific binding partner” is a member of a specific binding pair. A specific binding pair comprises two different molecules that specifically bind to each other through chemical or physical means. Therefore, in addition to antigen and antibody specific binding, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced.

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary or even deleterious, depending on the therapeutic objectives.

The term “antigen-binding portion” of a binding protein means one or more fragments of a binding protein (e.g., an antibody) that retain the ability to specifically bind to an antigen. The antigen-binding portion of a binding protein can be performed by fragments of a full-length antibody, as well as bispecific, dual specific, or multi-specific formats; specifically binding to two or more different antigens. Examples of binding fragments encompassed within the term “antigen-binding portion” of an binding protein include (i) an Fab fragment, a monovalent fragment consisting of the VL, VH, CL and CH1 domains; (ii) an F(ab)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which comprises a single variable domain; and (vi) an isolated complementarity determining region (CDR). Furthermore, although the two domains of the Fv fragment, VL and VH, encoded by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (VH—CH1-VH—CH1) which, together with complementary light chain polypeptides, form a pair of antigen binding regions.

The term “multivalent binding protein” means a binding protein comprising two or more antigen binding sites. In an embodiment, the multivalent binding protein is engineered to have three or more antigen binding sites, and is not a naturally occurring antibody. The term “multispecific binding protein” refers to a binding protein capable of binding two or more related or unrelated targets. In an embodiment, the dual variable domain (DVD) binding proteins provided herein comprise two or more antigen binding sites and are tetravalent or multivalent binding proteins.

The term “linker” means an amino acid residue or a polypeptide comprising two or more amino acid residues joined by peptide bonds that are used to link two polypeptides (e.g., two VH or two VL domains). Such linker polypeptides are well known in the art (see, e.g., Holliger et al. (1993) Proc. Natl. Acad. Sci. USA 90:6444-6448; Poljak et al. (1994) Structure 2:1121-1123).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “CDR” means a complementarity determining region within an immunoglobulin variable region sequence. There are three CDRs in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” means a region of an antigen that is bound by a binding protein, e.g., a polypeptide and/or other determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) known to bind to the complementary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (one prevents the binding or modulating effect of the other). In addition, structural definitions of epitopes (overlapping, similar, identical) are informative; and functional definitions encompass structural (binding) and functional (modulation, competition) parameters. Different regions of proteins may perform different functions. For example specific regions of a cytokine interact with its cytokine receptor to bring about receptor activation whereas other regions of the protein may be required for stabilizing the cytokine. To abrogate the negative effects of cytokine signaling, the cytokine may be targeted with a binding protein that binds specifically to the receptor interacting region(s), thereby preventing the binding of its receptor. Alternatively, a binding protein may target the regions responsible for cytokine stabilization, thereby designating the protein for degradation. The methods of visualizing and modeling epitope recognition are known to one skilled in the art (US 20090311253).

“Pharmacokinetics” refers to the process by which a drug is absorbed, distributed, metabolized, and excreted by an organism. To generate a multivalent binding protein molecule with a desired pharmacokinetic profile, parent monoclonal antibodies with similarly desired pharmacokinetic profiles are selected. The PK profiles of the selected parental monoclonal antibodies can be easily determined in rodents using methods known to one skilled in the art (US 20090311253).

“Bioavailability” refers to the amount of active drug that reaches its target following administration. Bioavailability is function of several of the previously described properties, including stability, solubility, immunogenicity and pharmacokinetics, and can be assessed using methods known to one skilled in the art (US 20090311253).

The term “surface plasmon resonance” means an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jönsson et al. (1993) Ann. Biol. Clin. 51:19-26. The term “Kₒₙ” means the on rate constant for association of a binding protein (e.g., an antibody or DVD-Ig™) to the antigen to form the, e.g., DVD-Ig™/antigen complex. The term “Kₒₙ” also means “association rate constant”, or “ka”, as is used interchangeably herein. This value indicating the binding rate of a binding protein to its target antigen or the rate of complex formation between a binding protein, e.g., an antibody, and antigen also is shown by the equation below:

Antibody (“Ab”)+Antigen (“Ag”)→*Ab−Ag

The term “K_{off}” means the off rate constant for dissociation, or “dissociation rate constant”, of a binding protein (e.g., an antibody or DVD-Ig™) from the, e.g., DVD-Ig™/antigen complex as is known in the art. This value indicates the dissociation rate of a binding protein, e.g., an antibody, from its target antigen or separation of Ab−Ag complex over time into free antibody and antigen as shown by the equation below:

Ab+Ag←Ab−Ag

The terms “K_{d}” and “equilibrium dissociation constant” means the value obtained in a titration measurement at equilibrium, or by dividing the dissociation rate constant (K_{off}) by the association rate constant (Kₒₙ). The association rate constant, the dissociation rate constant and the equilibrium dissociation constant, are used to represent the binding affinity of a binding protein (e.g., an antibody or DVD-Ig™) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The term “variant” means a polypeptide that differs from a given polypeptide in amino acid sequence by the addition (e.g., insertion), deletion, or conservative substitution of amino acids, but that retains the biological activity of the given polypeptide (e.g., a variant TfR antibody can compete with anti-TfR antibody for binding to TfR). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). The hydropathic index of an amino acid is based on a consideration of its hydrophobicity and charge. It is known in the art that amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptide or fragment thereof that has been differentially processed, such as by proteolysis, phosphorylation, or other post-translational modification, yet retains its biological activity or antigen reactivity, e.g., the ability to bind to TfR. The term “variant” encompasses fragments of a variant unless otherwise defined. A variant may be 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% identical to the wildtype sequence.

### I. Generation of Binding Proteins

Binding proteins capable of binding TfR and methods of making the same are provided. The binding protein can be generated using various techniques. Expression vectors, host cell and methods of generating the binding protein are provided and are well known in the art.

A. Generation of Parent Monoclonal Antibodies

The variable domains of the DVD binding protein can be obtained from parent antibodies, including polyclonal Abs and mAbs capable of binding antigens of interest. These antibodies may be naturally occurring or may be generated by recombinant technology. The person of ordinary skill in the art is well familiar with many methods for producing antibodies, including, but not limited to using hybridoma techniques, selected lymphocyte antibody method (SLAM), use of a phage, yeast, or RNA-protein fusion display or other library, immunizing a non-human animal comprising at least some of the human immunoglobulin locus, and preparation of chimeric, CDR-grafted, and humanized antibodies. See, e.g., US Patent Publication No. 20090311253 A1. Variable domains may also be prepared using affinity maturation techniques.

B. Criteria for Selecting Parent Monoclonal Antibodies

An embodiment is provided comprising selecting parent antibodies with at least one or more properties desired in the DVD binding protein molecule. In an embodiment, the desired property is one or more antibody parameters, such as, for example, antigen specificity, affinity to antigen, potency, biological function, epitope recognition, stability, solubility, production efficiency, immunogenicity, pharmacokinetics, bioavailability, tissue cross reactivity, or orthologous antigen binding. See, e.g., US Patent Publication No. 20090311253.

C. Construction of Binding Protein Molecules

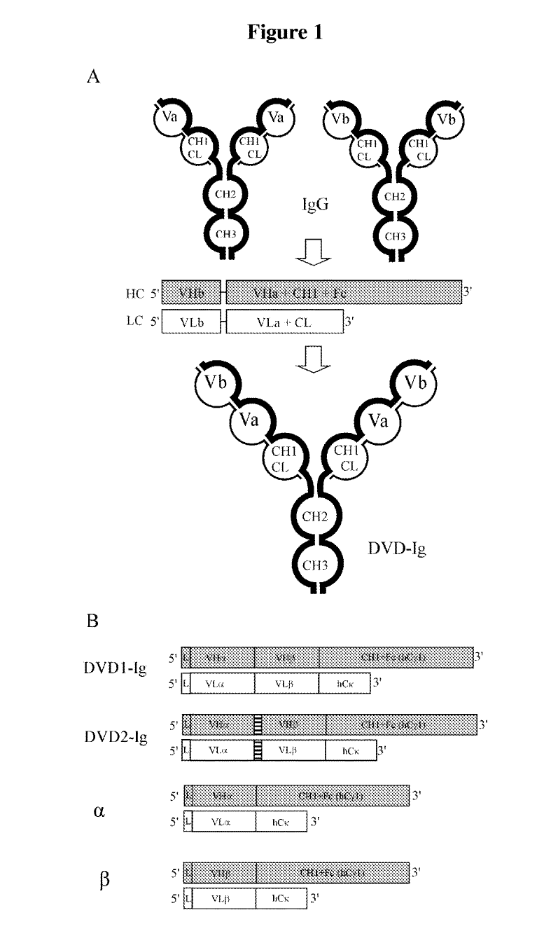

The binding protein may be designed such that two different light chain variable domains (VL) from the two different parent monoclonal antibodies are linked in tandem directly or via a linker by recombinant DNA techniques, followed by the light chain constant domain CL. Similarly, the heavy chain comprises two different heavy chain variable domains (VH) linked in tandem, directly or via a linker, followed by the constant domain CH1 and Fc region (FIG. 1).

The variable domains can be obtained using recombinant DNA techniques from parent antibodies generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

The linker sequence may be a single amino acid or a polypeptide sequence. In an embodiment, the choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from the C-terminus of a V domain and 4-6 residues from the N-terminus of a CL/CH1 domain. DVD binding proteins were generated using N-terminal 5-6 amino acid residues, or 11-12 amino acid residues, of CL or CH1 as a linker in the light chain and heavy chains, respectively. The N-terminal residues of CL or CH1 domains, particularly the first 5-6 amino acid residues, can adopt a loop conformation without strong secondary structures, and therefore can act as flexible linkers between the two variable domains. The N-terminal residues of CL or CH1 domains are natural extension of the variable domains, as they are part of the Ig sequences, and therefore their use minimizes to a large extent any immunogenicity potentially arising from the linkers and junctions.

In a further embodiment, of any of the heavy chain, light chain, two chain, or four chain embodiments, includes at least one linker comprising AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA(G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); or G/S based sequences (e.g., G4S repeats; SEQ ID NO: 29). In an embodiment, X2 is an Fc region. In another embodiment, X2 is a variant Fc region.

In various embodiments, the linker comprises GS-H10 (Chain H) GGGGSGGGGS (SEQ ID NO:178). In various embodiments, the linker comprises GS-L10 (Chain L) GGSGGGGSG (SEQ ID NO:179). In various embodiments, the linker comprises HG-short (Chain H) ASTKGP (SEQ ID NO:21). In various embodiments, the linker comprises LK-long (Chain L) TVAAPSVFIFPP (SEQ ID NO: 14). For example SEQ ID NOs: 21 and 178 are located on a variable heavy chain or domain of a DVD-Ig. For example SEQ ID NOs: 14 and 179 are located on a variable light chain or domain of a DVD-Ig.

Other linker sequences may include any sequence of any length of a CL/CH1 domain but not all residues of a CL/CH1 domain; for example the first 5-12 amino acid residues of a CL/CH1 domain; the light chain linkers can be from CK or CA; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR); G/S based sequences (e.g., G4S repeats; SEQ ID NO: 29); hinge region-derived sequences; and other natural sequences from other proteins.

In an embodiment, a constant domain is linked to the two linked variable domains using recombinant DNA techniques. In an embodiment, a sequence comprising linked heavy chain variable domains is linked to a heavy chain constant domain and a sequence comprising linked light chain variable domains is linked to a light chain constant domain. In an embodiment, the constant domains are human heavy chain constant domains and human light chain constant domains respectively. In an embodiment, the DVD heavy chain is further linked to an Fc region. The Fc region may be a native sequence Fc region or a variant Fc region. In another embodiment, the Fc region is a human Fc region. In another embodiment, the Fc region includes Fc region from IgG1, IgG2, IgG3, IgG4, IgA, IgM, IgE, or IgD.

In another embodiment, two heavy chain DVD polypeptides and two light chain DVD polypeptides are combined to form a DVD binding protein. Tables 1A-1C list amino acid sequences of VH and VL regions of exemplary antibodies useful for treating disease. In an embodiment, a DVD comprising at least two of the VH and/or VL regions listed in Table 1, in any orientation, is provided. In some embodiments, VD1 and VD2 are independently chosen. Therefore, in some embodiments, VD1 and VD2 comprise the same SEQ ID NO and, in other embodiments, VD1 and VD2 comprise different SEQ ID NOS. The VH and VL domain sequences provided below comprise complementarity determining regions (CDRs) and framework sequences that are either known in the art or readily discernible using methods known in the art. In some embodiments, one or more of these CDRs and/or framework sequences are replaced, without loss of function, by other CDRs and/or framework sequences from binding proteins that are known in the art to bind to the same antigen. Detailed description of specific DVD binding proteins capable of binding specific targets, and methods of making the same, is provided in the Examples section below.

D. Production of Binding Proteins

The binding proteins provided herein may be produced by any of a number of techniques known in the art. For example, expression from host cells, wherein expression vector(s) encoding the DVD heavy and DVD light chains is (are) transfected into a host cell by standard techniques. Although it is possible to express the DVD binding proteins provided herein in either prokaryotic or eukaryotic host cells, DVD binding proteins are expressed in eukaryotic cells, for example, mammalian host cells, because such eukaryotic cells (and in particular mammalian cells) are more likely than prokaryotic cells to assemble and secrete a properly folded and immunologically active DVD binding protein.

In an exemplary system for recombinant expression of DVD proteins, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. A method of synthesizing a DVD protein provided herein by culturing a host cell provided herein in a suitable culture medium until a DVD protein is synthesized is also provided. The method can further comprise isolating the DVD protein from the culture medium.

An important feature of DVD binding protein is that it can be produced and purified in a similar way as a conventional antibody. The production of DVD binding protein results in a homogeneous, single major product with desired dual-specific activity, without the need for sequence modification of the constant region or chemical modifications. Other previously described methods to generate “bi-specific”, “multi-specific”, and “multi-specific multivalent” full length binding proteins can lead to the intracellular or secreted production of a mixture of assembled inactive, mono-specific, multi-specific, multivalent, full length binding proteins, and multivalent full length binding proteins with a combination of different binding sites.

Surprisingly, the design of the “dual-specific multivalent full length binding proteins” provided herein leads to a dual variable domain light chain and a dual variable domain heavy chain that assemble primarily to the desired “dual-specific multivalent full length binding proteins”.

At least 50%, at least 75% and at least 90% of the assembled, and expressed dual variable domain immunoglobulin molecules are the desired dual-specific tetravalent protein, and therefore possess enhanced commercial utility. Thus, a method to express a dual variable domain light chain and a dual variable domain heavy chain in a single cell leading to a single primary product of a “dual-specific tetravalent full length binding protein” is provided.

Methods of expressing a dual variable domain light chain and a dual variable domain heavy chain in a single cell leading to a “primary product” of a “dual-specific tetravalent full length binding protein”, where the “primary product” is more than 50%, such as more than 75% and more than 90%, of all assembled protein, comprising a dual variable domain light chain and a dual variable domain heavy chain are provided.

E. DVD Cassettes

In certain embodiments, cassettes can be used to construct binding proteins that specifically bind to an antigen expressed on brain vascular epithelium of a subject that facilitates uptake of the binding protein into the brain of the subject. In some embodiments, the formula for these binding proteins is

Out1-(X1)m-In1-(X2)n (I)

According to Formula I, Out1 is a first outer binding domain and In1 is a first inner binding domain. In certain embodiments, the inner binding domain represents a binding domain positioned closer to the Fc region of a DVD-Ig™ than the outer binding domain. In other embodiments, the outer binding domain is located at or near the N-terminal end of the binding protein while the inner binding domain is located at or near the C-terminal end of the binding protein.

According to Formula I, X1 is a linker. According to some embodiments, X1 is any of the linkers defined herein. According to other specific embodiments, X1 has a sequence comprising the amino acid sequences of SEQ ID NO:14 or 21 when Out1 specifically binds an antigen expressed on brain vascular epithelium of a subject that facilitates uptake of the binding protein into the brain of the subject and In1 does not specifically bind said antigen, while X1 has a sequence comprising the amino acid sequence of SEQ ID NO:178 or 179 when In1 specifically binds an antigen expressed on brain vascular epithelium of a subject that facilitates uptake of the binding protein into the brain of the subject and Out1 does not specifically bind said antigen. According to Formula I, X2 is an Fc region. The values of m and n in Formula I are 0 or 1. In certain embodiments, when n is 0 X1 is X1 comprises the amino acid sequence of SEQ ID NO:14 or 179 depending on whether Out1 or In1 specifically binds an antigen expressed on brain vascular epithelium of a subject that facilitates uptake of the binding protein into the brain of the subject. When n is 1 X1 comprises the amino acid sequences of SEQ ID NO:21 or 178 depending on whether Out1 or In1 specifically binds said antigen.

When Out1 is used to specifically bind an antigen expressed on brain vascular epithelium of a subject that facilitates uptake of the binding protein into the brain it does not need to have as high an affinity as when In1 is used. Thus, in certain embodiments, when Out1 specifically binds an antigen expressed on brain vascular epithelium of a subject that facilitates uptake of the binding protein into the brain of the subject the binding affinity that Out 1 has for said antigen is lower than if In1 were to bind said antigen. For example, when Out1 specifically binds said antigen, the EC50 of the binding is greater than about 1, 2, 3, 4, 5, 6, 7, 8, 9 or 10 nM. In other embodiments, when Out1 specifically binds said antigen, the EC50 of the binding is between about 1 and 10 nM, 2 and 8 nM, 3 and 10 nM, 3 and 9 nM, 3 and 8 nM, 3 and 7nM, 3 and 6nM, 3 and 5 nM, 3 and 4nM, 4 and 10 nM or 5 and 10 nM

In other embodiments, when Out1 specifically binds transferrin receptor (TfR) Out1 has an affinity for TfR that is lower than if In1 were to specifically bind to TfR. For example, when Out1 specifically binds said antigen, the EC50 of the binding is greater than about 1, 2, 3, 4, 5, 6, 7, 8, 9 or 10 nM. In other embodiments, the EC50 is greater than about 3 nM. In other embodiments, when Out1 specifically binds TfR, the EC50 of the binding is between about 1 and 10 nM, 2 and 8 nM, 3 and 10 nM, 3 and 9 nM, 3 and 8 nM, 3 and 7 nM, 3 and 6 nM, 3 and 5 nM, 3 and 4 nM, 4 and 10 nM or 5 and 10 nM. In other embodiments, the EC50 is between about 3 and 10 nM, 3 and 9 nM, 3 and 8 nM, 3 and 7 nM, 3 and 6 nM, 3 and 5 nM, or 3 and 4 nM. In certain embodiments, Out1 comprises the amino acid sequence of SEQ ID NO:56.

In other embodiments, when Out1 specifically binds an antigen expressed on brain vascular epithelium of a subject that facilitates uptake of the binding protein into the brain, In1 binds another antigen. This antigen can be selected from CGRP, TNFα, RGMA, Substance P, Bradykinin, Nav1.7, LPA, P2X3, NGF, Abeta; BACE1; IL-1β; IGF1, or 2; IL-18; IL-6; RAGE; NGF; EGFR; cMet, Her-2 and CD-20.

When In1 is used to specifically bind an antigen expressed on brain vascular epithelium of a subject that facilitates uptake of the binding protein into the brain it needs to have a higher affinity than when Out1 is used. Thus, in certain embodiments, when In1 specifically binds an antigen expressed on brain vascular epithelium of a subject that facilitates uptake of the binding protein into the brain of the subject the binding affinity that In1 has for said antigen is higher than if Out1 were to bind said antigen. For example, when In1 specifically binds said antigen, the EC50 of the binding is less than about 1, 2, 3, 4, 5, 6, 7, 8, 9 or 10 nM. In other embodiments, when Out1 specifically binds said antigen, the EC50 of the binding is between about 1 and 0.001 nM, 2 and 0.001 nM, 3 and 0.0001 nM, 3 and 0.001 nM, 3 and 0.01 nM, 3 and 0.1 nM, 3 and 1 nM, 3 and 5nM, 3 and 10nM, 4 and 10nM or 5 and 10 nM.

In other embodiments, when Out1 specifically binds transferrin receptor (TfR) In1 has an affinity for TfR that is higher than if Out1 were to specifically bind to TfR. For example, when In1 specifically binds said antigen, the EC50 of the binding is less than about 1, 2, 3, 4, 5, 6, 7, 8, 9 or 10 nM. In other embodiments, the EC50 is less than about 3 nM. In other embodiments, when In1 specifically binds TfR, the EC50 of the binding is between about 1 and 0.001 nM, 2 and 0.001 nM, 3 and 0.0001 nM, 3 and 0.001 nM, 3 and 0.01 nM, 3 and 0.1 nM, 3 and 1 nM, 3 and 5 nM, 3 and 10 nM, 4 and 10 nM or 5 and 10 nM. In other embodiments, the EC50 is between about 3 and 0.0001 nM, 3 and 0.001 nM, 3 and 0.01 nM, 3 and 0.1 nM, 3 and 1 nM, 3 and 5 nM or 3 and 10 nM. In certain embodiments, In1 comprises the amino acid sequence of SEQ ID NO:36.

In other embodiments, when In1 specifically binds an antigen expressed on brain vascular epithelium of a subject that facilitates uptake of the binding protein into the brain, Out1 binds another antigen. This antigen can be selected from CGRP, TNFα, RGMA, Substance P, Bradykinin, Nav1.7, LPA, P2X3, NGF, Abeta; BACE1; IL-1β; IGF1, or 2; IL-18; IL-6; RAGE; NGF; EGFR; cMet, Her-2 and CD-20.

In other embodiments, a binding protein may comprise a second binding protein. In some embodiments, the formula for this second binding protein is

Out2-(X1)m-In2-(X2)n (II)

According to Formula II, Out2 is a second outer binding domain and In2 is a second inner binding domain. As explained above. in certain embodiments, the inner binding domain represents a binding domain positioned closer to the Fc region of a DVD-Ig™ than the outer binding domain. In other embodiments, the outer binding domain is located at or near the N-terminal end of the binding protein while the inner binding domain is located at or near the C-terminal end of the binding protein. X1 and X2 are as defined in Formula I, above.

Out2 and In2 operate in the same manner as Out1 and In1 described above. This second binding protein can be associated with a first binding protein to form a binding polypeptide such as a DVD-Ig™. In these embodiments, in the first binding protein n is 1 and in the second binding protein n is 0. In certain embodiments, both Out1 and Out2 bind an antigen expressed on brain vascular epithelium of a subject that facilitates uptake of the binding protein into the brain of the subject. In other embodiments, both In1 and In2 bind said antigen. According to other embodiments, Out1 and In2 or Out2 and In1 bind said antigen. In certain embodiments, Out2 comprises the amino acid sequence of SEQ ID NO:37. In other embodiments, In2 comprises the amino acid sequence of SEQ ID NO:57.

### II. Uses of Binding Proteins

Given their ability to bind to two or more antigens the binding proteins provided herein can be used to detect the antigens (e.g., in a biological sample, such as serum or plasma), using a conventional immunoassay, such as an enzyme linked immunosorbent assays (ELISA), a radioimmunoassay (RIA), or tissue immunohistochemistry. The binding protein is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol and examples of suitable radioactive materials include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, and ¹⁵³Sm.

In an embodiment, the binding proteins provided herein are capable of neutralizing the activity of their antigen targets both in vitro and in vivo. Accordingly, such binding proteins can be used to inhibit antigen activity, e.g., in a cell culture containing the antigens, in human subjects or in other mammalian subjects having the antigens with which a binding protein provided herein cross-reacts. In another embodiment, a method for reducing antigen activity in a subject suffering from a disease or disorder in which the antigen activity is detrimental is provided. A binding protein provided herein can be administered to a human subject for therapeutic purposes.

The term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins provided herein include those disorders discussed below and in the section pertaining to pharmaceutical compositions comprising the binding proteins.

DVD binding proteins are useful as therapeutic agents to simultaneously block two different targets to enhance efficacy/safety and/or increase patient coverage.

Additionally, DVD binding proteins provided herein can be employed for tissue-specific delivery (target a tissue marker and a disease mediator for enhanced local PK thus higher efficacy and/or lower toxicity), including intracellular delivery (targeting an internalizing receptor and an intracellular molecule), delivering to inside brain (targeting transferrin receptor and a CNS disease mediator for crossing the blood-brain barrier). DVD binding protein can also serve as a carrier protein to deliver an antigen to a specific location via binding to a non-neutralizing epitope of that antigen and also to increase the half-life of the antigen. Furthermore, DVD binding protein can be designed to either be physically linked to medical devices implanted into patients or target these medical devices (see Burke et al. (2006) Advanced Drug Deliv. Rev. 58(3): 437-446; Hildebrand et al. (2006) Surface and Coatings Technol. 200(22-23): 6318-6324; Drug/device combinations for local drug therapies and infection prophylaxis, Wu (2006) Biomaterials 27(11):2450-2467; Mediation of the cytokine network in the implantation of orthopedic devices, Marques (2005) Biodegradable Systems in Tissue Engineer. Regen. Med. 377-397). Briefly, directing appropriate types of cell to the site of medical implant may promote healing and restoring normal tissue function. Alternatively, inhibition of mediators (including but not limited to cytokines), released upon device implantation by a DVD coupled to or target to a device is also provided.

A. Use of Binding Proteins in Various Diseases

Binding protein molecules provided herein are useful as therapeutic molecules to treat various diseases, e.g., wherein the targets that are recognized by the binding proteins are detrimental. Such binding proteins may bind one or more targets involved in a specific disease. Binding to BBB receptors (e.g., TfR) has also been shown to enhance penetration of the blood brain barrier and thus is useful for delivering therapeutics to the brain. Without limiting the disclosure, further information on certain disease conditions is provided.

a. Neurodegenerative Diseases

Neurodegenerative diseases are either chronic in which case they are usually age-dependent or acute (e.g., stroke, traumatic brain injury, spinal cord injury, etc.). They are characterized by progressive loss of neuronal functions (e.g., neuronal cell death, axon loss, neuritic dystrophy, demyelination), loss of mobility and loss of memory. These chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments fail to stop disease progression. Specific therapies targeting more than one disease mediator may provide even better therapeutic efficacy for chronic neurodegenerative diseases than observed with targeting a single disease mechanism (see Deane et al. (2003) Nature Med. 9:907-13; and Masliah et al. (2005) Neuron. 46:857).

The binding protein molecules provided herein can allow for transport of therapeutics across the blood brain barrier. In certain embodiments, these therapeutics bind one or more targets involved in chronic neurodegenerative diseases such as Alzheimer's disease. The efficacy of binding protein molecules and its combination with other therapeutics can be validated in pre-clinical animal models such as the transgenic mice that over-express amyloid precursor protein or RAGE and develop Alzheimer's disease-like symptoms. In addition, binding protein molecules can be constructed and tested for efficacy in the animal models and the best therapeutic binding protein can be selected for testing in human patients. Binding protein molecules can also be employed for treatment of other neurodegenerative diseases such as Parkinson's disease. Other pain related targets include CGRP, TNFα, RGMA, Substance P, Bradykinin, Nav1.7, LPA, P2X3, and NGF.

b. Neuronal Regeneration and Spinal Cord Injury

Despite an increase in knowledge of the pathologic mechanisms, spinal cord injury (SCI) is still a devastating condition and represents a medical indication characterized by a high medical need. Most spinal cord injuries are contusion or compression injuries and the primary injury is usually followed by secondary injury mechanisms (inflammatory mediators e.g., cytokines and chemokines) that worsen the initial injury and result in significant enlargement of the lesion area, sometimes more than 10-fold.

The efficacy of binding protein molecules can be validated in pre-clinical animal models of spinal cord injury. In addition, these binding protein molecules can be constructed and tested for efficacy in the animal models and the best therapeutic binding protein can be selected for testing in human patients. In general, antibodies do not cross the blood brain barrier (BBB) in an efficient and relevant manner. However, in certain neurologic diseases, e.g., stroke, traumatic brain injury, multiple sclerosis, etc., the BBB may be compromised and allows for increased penetration of binding proteins and antibodies into the brain. In other neurological conditions, where BBB leakage is not occurring, one may employ the targeting of endogenous transport systems, including carrier-mediated transporters such as glucose and amino acid carriers and receptor-mediated transcytosis-mediating cell structures/receptors at the vascular endothelium of the BBB, thus enabling trans-BBB transport of the binding protein. Structures at the BBB enabling such transport include but are not limited to the insulin receptor, e.g., human insulin receptor (HIR), transferrin receptor, LRP family, IGFR, EPCR, EGFR, TNFR, Leptin receptor, M6PR, Neuronal nicotinic acetylcholine receptor, Lipoprotein receptor, AchR, DTr, Glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, TMEM30A, and RAGE. In addition, strategies enable the use of binding proteins also as shuttles to transport potential drugs into the CNS including low molecular weight drugs, nanoparticles and nucleic acids (Coloma et al. (2000) Pharm Res. 17(3):266-74; Boado et al. (2007) Bioconjug. Chem. 18(2):447-55).

c Other Disease Targets

Other disease targets or disease conditions which may be treated with the binding molecules of the invention are disclosed in US 2009-0304693A1 and US 2010-0076178A1, each of which are specifically incorporated by reference herein in their entireties. For example, a binding protein of the invention suitable for neurological use may bind at least one target antigen selected from the group consisting of Abeta; TNF-alpha; BACE1; IL-1.beta; IGF1,2; IL-18; IL-6; RAGE; NGF; EGFR; CD-20 and RGMA. In other exemplary embodiments, a binding protein of the disclosure is suitable for anti-cancer use and binds at least one target antigen selected from the group consisting of CD-20; CD-19; CD-80; CD-22; CD-40; CD-3; HER-2; EGFR; IGF1,2; IGF1R; RON; HGF; c-MET; VEGF; DLL4; NRP1; PLGF; and ErbB3.

d. Pain Modulation

Brain pathways governing the perception of pain and the signals sent to and received from the body still not completely understood. Junctions in the spinal cord are involved in the relay and modulation of sensations of pain to various regions of the brain, including the periaqueductal grey region (Ugeer, P. L., Eccles, J. C., and Ugeer, E. G. (1987). Molecular Neurobiology of the Mammalian Brain, Plenum Press, New York).

Pain can be classified as either acute or chronic. Acute pain can be caused by damage to tissue and generally has a sudden onset and a limited duration. Chronic pain tends to last longer than acute pain and is usually associated with a long-term illness. It is usually more resistant to treatment, and can be the defining characteristic of a disease (such as fibromyalgia). It can be the result of damaged tissue, but more often is attributed to nerve damage. Pain can also be classified by the kind of damage that causes it. Nociceptive pain is pain caused by tissue damage, while neuropathic pain is pain caused by nerve damage. Nociceptive pain may be further divided into three different sub-categories: visceral, deep somatic, and superficial somatic pain.

Examples of pain include but are not limited to: acute pain, chronic pain, muscle pain, joint pain, chest pain, neck pain, shoulder pain, hip pain, abdominal pain, carpal tunnel syndrome, knee pain, back pain, myofascial pain syndrome, fibromyalgia, arthritic pain, headache (e.g., a migraine headache), Piriformis syndrome, whiplash, chronic muscle pain, nociceptive pain, visceral pain, deep somatic pain, superficial somatic pain, neuropathic pain, central pain syndrome, complex regional pain syndrome, diabetic peripheral neuropathy, pain associated with shingles, postherpetic neuralgia, neuralgia, trigeminal neuralgia, sciatica pain, arachnoiditis (spinal pain), central pain syndrome, phantom limb pain, phantom body pain, neuropathy, compartment syndrome, acute herpetic pain, post herpetic pain, causalgia pain, idiopathic pain, inflammatory pain, cancer pain, postoperative pain, interstitial cystitis pain, irritable bowel syndrome (IBS), tendinitis, breakthrough pain, and incident pain.

Neuropathic pain is a particular type of chronic pain that has a complex and variable etiology. It is frequently a chronic condition attributable to complete or partial transection of a nerve, trauma or injury to a nerve, nerve plexus or soft tissue, or other conditions, including cancer, AIDS and idiopathic causes. Neuropathic pain is characterized by hyperalgesia (lowered pain threshold and enhanced pain perception) and by allodynia (pain from innocuous mechanical or thermal stimuli). The condition is often progressive in nature. Because the hyperesthetic component of neuropathic pain does not respond to the same pharmaceutical interventions as does more generalized and acute forms of pain, development of effective long-term treatment modalities has been problematic.

Psychogenic pain is a condition associated or correlated with a psychological, emotional, or behavioral stimulus. Thus, the physical pain that is of psychological origin. Headaches, muscle pains, back pain, and stomach pains are some of the most common types of psychogenic pain observed in subjects.

Analgesia, or the reduction of pain perception, can be attained by many methods including directly decreasing transmission along such nociceptive pathways by using for example opiates, and inhibiting release of neurotransmitters (See U.S. Pat. No. 8,268,774, which is incorporated by reference, herein, in its entirety).

Without being limited by any particular theory or mechanism of action, it is here envisioned that binding proteins or peptides described herein are effective delivery vehicles for an agent (e.g., therapeutic and diagnostic) for treatment of pain. A pharmaceutical composition used for treatment of a subject comprises the binding protein or peptide herein; and at least one therapeutic agent. The composition efficiently penetrates the BBB, and is effective for treatment and/or modulation of pain.

In certain embodiments, the binding protein is a bispecific molecule for example a bispecific DVD-Ig as described herein. In various embodiments, the DVD-Ig has at least one binding region that specifically binds a BBB receptor, antigen or target. For example, the BBB receptor, antigen or target comprises an insulin receptor, a transferrin receptor, a LRP, a melanocortin receptor, a nicotinic acetylcholine receptor, a VACM-1 receptor, a vascular endothelial growth factor, a glucocorticoid receptor, an ionotropic glutamate receptor, a M3 receptor, an aryl hydrocarbon receptor, a GLUT-1, an inositol-1,4,5-trisphosphate (IP3) receptor, a N-methyl-D-aspartate receptor, a S1P1, a P2Y receptor and a Receptor for Advanced Glycation Endproducts (RAGE) receptor.

In various embodiments, the pharmaceutical composition includes the binding protein or peptide, and a detectable agent. In various embodiments, the detectable agent comprises a detectable agent or imaging agent for analysis of the brain. For example the detectable agent comprises a fluorescent agent, a colorimetric agent, an enzymatic agent, or a radioactive agent.

### III. Pharmaceutical Compositions

Pharmaceutical compositions comprising one or more binding proteins, either alone or in combination with prophylactic agents, therapeutic agents, and/or pharmaceutically acceptable carriers are provided. The pharmaceutical compositions comprising binding proteins provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art (US Patent Publication No. 20090311253 A1).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (US Patent Publication No. 20090311253 A1).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-20 mg/kg, for example, 1-10 mg/kg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### IV. Combination Therapy

A binding protein provided herein also can also be administered with one or more additional medicaments or therapeutic agents useful in the treatment of various diseases, the additional agent being selected by the skilled artisan for its intended purpose. For example, the additional agent can be a therapeutic agent art-recognized as being useful to treat the disease or condition being treated by the antibody provided herein. The combination can also include more than one additional agent, e.g., two or three additional agents.

The binding agent in various embodiments is administered with an agent that is a protein, a peptide, a carbohydrate, a drug, a small molecule, and a genetic material (e.g., DNA or RNA). In various embodiments, the agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteriod, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

The additional agent in various embodiments is a therapeutic agent. In various embodiments, the therapeutic agent comprises budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody specific against or an agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1RI, sIL-1RII, sIL-6R, an anti-inflammatory cytokine, IL-4, IL-10, IL-11, IL-13, or TGFβ.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

### V. Diagnostics

The disclosure herein also provides diagnostic applications including, but not limited to, diagnostic assay methods, diagnostic kits containing one or more binding proteins, and adaptation of the methods and kits for use in automated and/or semi-automated systems. The methods, kits, and adaptations provided may be employed in the detection, monitoring, and/or treatment of a disease or disorder in an individual. This is further elucidated below.

A. Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure (US 2009-0311253 A1).

B. Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art (US Patent Publication No. 2009-0311253 A1).

C. Adaptation of Kit and Method

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other platforms available from Abbott Laboratories include, but are not limited to, AxSYM®, IMx® (see, for example, U.S. Pat. No. 5,294,404, PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. Nos. 5,063,081, 7,419,821, and 7,682,833; and US Publication Nos. 20040018577, 20060160164 and 20090311253.

It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### Generation and Characterization of Dual Variable Domain (DVD) Binding Proteins

Four-chain dual variable domain (DVD)-Igbinding proteins using parent antibodies with known amino acid sequences were generated by synthesizing polynucleotide fragments encoding DVD binding protein variable heavy and DVD binding protein variable light chain sequences and cloning the fragments into a pHybE-D2 vector according to art known methods. The DVD binding protein constructs were cloned into and expressed in 293 cells and purified according to art known methods. DVD VH and VL chains for the DVD binding proteins, as well as selected CDR sequences are provided below.

TABLE 2 List of Amino Acid Sequences of VH and VL Regions of Antibodies for
Generating Binding Proteins, Including Multivalent Binding Proteins (CDRs highlighted)

| | | | |
|---|---|---|---|
| SEQ ID | Accession | Protein | Sequence |
| | No. | region | |
| | | | EVQLVESGGGLVQPGGSLRL S CAASGFTFSNYGMHW I RQAPGKG |
| 30 | AB402VH | VH-Tf R | LEW IAMIYYDS SKMNYADTVKGRE T I SRDNAKNSLYLQMNSLRA |
| | | | EDTAVYYCAVPTSHYVVDVWGQGTTVTVS S |
| | | | AI QMTQ SP S SL SASVGDRVT I TCQASQDIGNWLAWYQQKPGKSP |
| 31 | AB402VL | VL-Tf R | KLL I YGATSLADGVP SRF S GSRS GT DE TLTI SS LQPEDFATYYC |
| | | | LQAYNTPWTF GGGTKVE I KR |
| | | | EVQLVESGGGLVUGGS LRLS CAASGFTFSNYGMHW I RQAPGKG |
| 32 | AB4O3VH | VH-Tf R | L EW TAMIYYDS SKMNYADTVKGRF T I SRDNAKNS LYLQMNSLRA |
| | | | EDTAVYYCAVP TSHYVVDVWGQGT TVTVSS |
| | | | E IVMTQ SPATL SVSPGERATL S CQASQDIGNWLAWYQUPGQ SR |
| 33 | AB403VL | VL-Tf R | RLL I YGATSLADGVPARF S. GSRS GTEFTLT I SS LQ $ EDFAVYYC |
| | | | WAYNITVITFGGGTRVE 'RR |
| | | | QVQLVQ S GAEVKKPGASVKVS CKASGFTFSNYGMHWI RQAPGQG |
| 34 | AB404VH | VH-Tf R | LEW IAMIYYDS SKMNYADTVKGRF TI TRDNS TNT L YME L S SLRS |
| | | | EDTAVYYCAVPTSHYVVDVWGQGTTVTVSS |
| | | | AI QMTQ SPS S L SASVGDRVT I TCQASQDIGNWLAWYQQKPGKSP |
| 35 | AB404VL | VL-Tf R | KLL I YGATSLADGVP SRF S GSRS GT DF TLT I S S LQPE DFAT YYC |
| | | | LQAYNTPWTFGGGTKVE I KR |
| | | | QVQ IJVQ S GAEVRRP GAS VICVS CRAS GF TFSNYGMHW I RQAPGQG |
| 6 | A B40 VH | VH-Tf R | LEWIAMIYYDSSKMNYADTVKGRET I TRDNSTNTLYMEL SSLRS |
| | | | EDTAVYYCAVFISHYVVDVWGQGTTVTVS S |
| | | | E IVMTOPATL SVSPGERATL S QASQDIGNWLAWYWKRGOP |
| 37 | AB405VL | VL-Tf R | RLL I YGATSLADGVPARF S GSR GTEF TLT I SS LQ S EDFAVYYC |
| | | | WAYNTFWTFGGGTRVE 'RR |
| | | | EVQL LE S GGGLVQPGGS LRL SCAASGFTFSNYGMSWVRQAPGKG |
| 38 | AB043VH | VH-APP | LEWVASIRSGGGRTYYSDNVKGRFT I SRDNSKNTLYLQMNSLRA |
| | | | EDTAVYYCVRYDHYSGSSDYWGQGTLVTVSS |
| | | | DVVMTQSPL SLPVTPGEPAS I SCKSSQSLLDSDGKTYLNWLLQK |
| 39 | AB043VL | VL-APP | PGQSPQRL I YLVSKLDS GVPDRF S GS GS GT DF T LK I SRVEAE DV |
| | | | GVYYCWQGTHFPRTFGQGTKVE I KR |
| | | | EVQLVE S GGGLVQPGNS LA T L S CVASGFTFSNYGMHWI RQARKKG |
| 56 | AB221VH | VH-TfR | LEINIAMIYYDSSKMNYADTVKGRFT I $RDN$KNTLYLEMNSLRS |
| | | | EDTAMYYCAVPTSHYVVDVWGQGVSVTVS S |
| | | | D I QMTQ SPAS L SAS LEE I VT I TCQASQDIGNWLAWYQQKPGKSF |
| 57 | AB221VL | VL-Tf R | QLL I YGATSLADGVP SRF S GSRS GTQF S LK I SRVQVED I GI YYC |
| | | | LQAYNTPWTEGGGTKLELKR |
| | | | EVQLVESGGGLVQPGGSLRL SCAASGFNIKDTYIHWVRQAPGKG |
| 58 | AB004VH | VH-Her2 | LEWVARIYPTNGYTRYADSVKGRFT I SADT SKNTAYLQMNSLRA |
| | | | EDTAVYYCSRWGGDGFYAMDYWGQGTLVTVSS |
| | | | D I QMTQ SPS S L SASVGDRVT I TCRASQDVNTAVAWYQQKPGKAP |
| 59 | AB004VL | VL-Her2 | KLL IYSASFLYSGVPSRF SGSRSGTDFTLT I S SLQPEDFATYYC |
| | | | QQHYTTPPTFGQGTKVE I KR |
| | | | D I QMTQ SPS S L SASVGDRVT I TCRASQDVSTAVAWYQQKPGKAP |
| 87 | BACE001VL | BACE1- VL | KLLIYSASFLYSGVPSRFSGSGSGTDFTLTISSLQPEDFATYYC |
| | | | QQSYTTPPTFGQGTKVE I KR |
| | | | D I QMTQ SPS S L SASVGDRVT I TCRASQDVSTAVAWYQQKPGKAP |
| 88 | BACE002VL | BACE1-VL | KLLIYSASFLYSGVPSRFSGSGSGTDFTLTISSLQPEDFATYYC |
| | | | QQFPTYLPTFGQGTKVE I KR DIQMTQSPSS |
| | | | D I QMTQ SPS S L SASVGDRVT I TCRASQDVSTAVAWYQQKPGKAP |
| 89 | BACE003VL | BACE1- VL | KLLIYSASFLYSGVPSRFSGSGSGTDFTLTISSLQPEDFATYYC |
| | | | QQGYNDPPTFGQGTKVE I KR DIQMTQSPSS |
| | | | D I QMTQ SPS S L SASVGDRVT I TCRASQDVSTAVAWYQQKPGKAP |
| 90 | BACE004VL | BACE1- VL | KLLIYSASFLYSGVPSRFSGSGSGTDFTLTISSLQPEDFATYYC |
| | | | QQSSTDPTTFGQGTKVE I KR DIQMTQSPSS |
| | | | D I QMTQ SPS S L SASVGDRVT I TCRASQVVANSLAWYQQKPGKAP |
| 91 | BACE005VL | BACE1-VL | KLL I YLASFLYSGVPSRF SGSGSGTDFTLT I S SLQPEDFATYYC |
| | | | QQDATSPPTFGQGTKVE I KR DIQMTQSPSS |
| | | | D I QMTQ SPS S L SASVGDRVT I TCRASQDVSTAVAWYQQKPGKAP |
| 92 | BACE006VL | BACE1- VL | KLLIYSASFLYSGVPSRFSGSGSGTDFTLTISSLQPEDFATYYC |
| | | | QQYATDPPTFGQGTKVE I KR DIQMTQSPSS |
| | | | EVQLVESGGGLVQPGGSLRL SCAASGFTFSGYAIHWVRQAPGKG |
| 93 | BACE001VH | BACE1-VH | LEWVGWISPAGGSTDYADSVKGRF T I SADT S KNTAYLQMN S LRA |
| | | | EDTAVYYCARGPFSPWVMDYWGQGTLVTVS S |
| | | | EVQLVESGGGLVQPGGSLRL SCAASGFTFLGYGIHWVRQAPGKG |
| 94 | BACE002VH | BACE1-VH | LEWVGWISPAGGSTDYADSVKGRF T I SADT S KNTAYLQMN S LRA |
| | | | EDTAVYYCARGPFSPWVMDYWGQGTLVTVS S |
| | | | EVQLVESGGGLVQPGGSLRL SCAASGFTFSGYAIHWVRQAPGKG |
| 95 | BACE003VH | BACE1-VH | LEWVGWISPAGGSTDYADSVKGRF T I SADT S KNTAYLQMN S LRA |
| | | | EDTAVYYCARGPFSPWVMDYWGQGTLVTVS S |
| | | | EVQLVESGGGLVQPGGSLRL SCAASGFTFSGYAIHWVRQAPGKG |
| 96 | BACE004VH | BACE1-VH | LEWVGWISPAGGSTDYADSVKGRF T I SADT S KNTAYLQMN S LRA |
| | | | EDTAVYYCARGPFSPWVMDYWGQGTLVTVS S |
| | | | EVQLVESGGGLVQPGGSLRL SCAASGFTFSGYAIHWVRQAPGKG |
| 97 | BACE005VH | BACE1-VH | LEWVGWISPAGGSTDYADSVKGRF T I SADT S KNTAYLQMN S LRA |
| | | | EDTAVYYCARGPFSPWVMDYWGQGTLVTVS S |
| | | | EVQLVESGGGLVQPGGSLRL SCAASGFTFSGYAIHWVRQAPGKG |
| 98 | BACE006VH | BACE1-VH | LEWVGWISPAGGSTDYADSVKGRF T I SADT S KNTAYLQMN S LRA |
| | | | EDTAVYYCARGPFSPWVMDYWGQGTLVTVS S |
| | | | EVQLVESGGGLVQPGGSLRL SCAASGFTFSRYSMSWVRQAPGKG |
| | | | LE LVAQINSVGNSTYYPDTVKGRF T I S RDNAKNT LYLQMN S LRA |
| 99 | ABETA001VH | VH-Abeta | EDTAVYYCASGDYWGQGTLVTVS S |
| | | | DVVMTQSPL SLPVTLGQPAS I S CRSSQSLIYSDGNAYLHWF LQK |
| | | | PGQSPRLL I YKVSNRFSGVPDRF S GS GS GT DF T LK I SRVEAEDV |
| 100 | ABETA001VL | VL-Abeta | GVYYCSQSTHVPWTFGQGTKVE I KR |
| | | | QVQLVESGGGLVQPGGSLRL SCAASGFTFSSYAMSWVRQAPGKG |
| 101 | ABETA002VH | VH-Abeta | LEWVSAINASGTRTYYADSVKGRFT I S RDN S KNT LYLQMN S LRA |
| | | | E D TAVYY CARGKGNTHKPYGYVRYFDVWGQ GT LVTVS S |
| | | | D IVL TQ SPAT L SL SPGERATL SCRASQSVSSSYLAWYQQKPGQA |
| 102 | ABETA002VL | VL-Abeta | PRLL I YGASSRATGVPARF S GS GS GT DF TL T I S S LEPEDFATYY |
| | | | CLQIYNMP ITFGQGTKVE I KRT |
| | | | EVQLQASGGGLVQAGGSLRL S CAASGFKITHYTMGWFRQAPGKE |
| 103 | VHH- | | REFVSRITWGGDNTFYSNSVKGRF T I SRDNAKNTVYLQMNSLKP |
| | | | TM TMEM30ATMEM30A EDTADYYCAAGSTSTATPLRVDYWGKGTQVTVS S |
| | | | QVQLVQSGAEVKKPGS SVKVSCKASGYTFTNYDIHWVRQAPGQG |
| 104 | HIR-VH | HIR-VH | LEWMGWIYPGDGSTKYNEKFKGRVT I TADESTSTAYMEL S SLRS |
| | | | E D TAVYY CARE WAYWGQGT TVTVS S |
| | | | DIQMTQSPSSLSASVGDRVTITCRASQDIGGNLYWYQQKPGKAP |
| 105 | HIR1-VL | HIR-VL | KLL I YATSSLDSGVP SRF S GSRS GT DYT LT I SS LQPEDFATYYC |
| | | | LQYSSSPWTFGQGTKVE I KR |
| | | | D I QMTQ SP S SL SASVGDRVT I TCRASQDIGGNLYWLQQKPGKAP |
| 106 | HIR2-VL | HIR-VL | KRL I YATSSLDSGVPKRF S GSRS GS DYT LT I SS LQPEDFATYYC |
| | | | LQYSSSPWTFGQGTKVE I KR |
| | | | D I QMTQ SP S SL SASVGDRVT I TCRASQD IGGNLYWL QQKP GKT 1 |
| 107 | HIR3-VL | HIR-VL | KRL I YATSSLDSGVP SRF S GS GS GT DYT LT I SS LQPEDFATYYC |
| | | | LQYSSSPWTFGQGTKVE I KR |
| | | | DIQMTQSPSSLSASVGDRVTITCRASQDIGGNLYWYQQKPGKAP |
| 108 | HIR4-VL | HIR-VL | KLL I YATSSLDSGVP SRF S GS GS GT DF TL T I S S LQPEDFATYYC |
| | | | LQYSSSPWTFGQGTKVE I KR |
| | | | EFQLQQSGPELVKPGASVRI S CKAS GY SF TDYNMNWVKQ SNGKS |
| 162 | 8C11-VH | VH | LEWVGVINPNYGSSTYNQKFKGKATL TVDQS S S TAYMQLNSLTS |
| | | | E D SAVYYCARKWGQLGRGFFDVWGT GT TVTVS S |
| | | | QIVL S Q SPAT L SASPGEKVTMTCRASSSVSYMHWFQQKPGS SPK |
| 163 | 8C11-VL | VL | PW I YATSNLASGVPARF S GS GS GT SYS L T I SRVEAEDAATYYCQ |
| | | | QWSSSPLTFGAGTKLELKR |
| | | | EVQLVQSGAEVKKPGASVKVSCKASGYTFTSHGISWVRQAPGQG |
| | | | L DWMGWI SPYSGNTNYAQKLQGPVTMT T DT S T S TAYMEL S SLRS |
| 170 | RGMA-J | L3-VHVH | EDTAVYYCARVGSGPYYYMDVWGQGTLVTVS S |
| | | | Q SAL TQPRSVS GSPGQ SVT I S CTGTSSSVGDS IYVSWYQQHPGK |
| 171 | RGMA-J | L3-VLVL | APKLMLYDVTKRPSGVPDRF S GSKS GNTAS LT I S GLQAEDEADY |
| | | | YCCSYAGTDTLFGGGTKVTVLG |

TABLE 3
CDR Sequences

| Accession No. | CDR | Sequence | SEQ ID NO: |
|---|---|---|---|
| AB402VH | CDR1 | GFTFSNYGMH | 76 |
| | CDR2 | MIYYDSSKMNYADTVKG | 77 |
| | CDR3 | PTSHYVVDV | 78 |
| AB402VL | CDR1 | QASQDIGNWLA | 79 |
| | CDR2 | GATSLAD | 80 |
| | CDR3 | LQAYNTPWT | 81 |
| AB403VH | CDR1 | GFTFSNYGMH | 76 |
| | CDR2 | MIYYDSSKMNYADTVKG | 82 |
| | CDR3 | PTSHYVVDV | 83 |
| AB403VL | CDR1 | QASQDIGNWLA | 84 |
| | CDR2 | GATSLAD | 85 |
| | CDR3 | LQAYNTPWT | 86 |
| AB404VH | CDR1 | GFTFSNYGMH | 76 |
| | CDR2 | MIYYDSSKMNYADTVKG | 77 |
| | CDR3 | PTSHYVVDV | 78 |
| AB405VH | CDR1 | GFTFSNYGMH | 76 |
| | CDR2 | MIYYDSSKMNYADTVKG | 77 |
| | CDR3 | PT S HYVVDV | 78 |
| AB405VL | CDR1 | QASQDIGNWLA | 79 |
| | CDR2 | GATSLAD | 80 |
| | CDR3 | LQAYNTPWT | 86 |
| AB043VH | CDR1 | GFTFSNYGMS | 109 |
| | CDR2 | s I RS GGGRTYYS DNVKG | 110 |
| | CDR3 | YDHYS GS SDY | 111 |
| AB043VL | CDR1 | KSSQSLLDSDG | 112 |
| | CDR2 | LVSKL DS | 113 |
| | CDR3 | WQGTHFPRT | 114 |
| AB221VH | CDR1 | GFTFSNYGMH | 115 |
| | CDR2 | MI YYDS SKMNYADTVKG | 116 |
| | CDR3 | PT S HYVVDV | 117 |
| AB221VL | CDR1 | QASQDIGNWLA | 118 |
| | CDR2 | GATSLAD | 119 |
| | CDR3 | LQAYNTPWT | 120 |
| ABOO4VH | CDR1 | GFN IKDTY I H | 121 |
| | CDR2 | RI YPTNGYTRYADSVKG | 122 |
| | CDR3 | WGGDGFYAMDY | 123 |
| ABOO4VL | CDR1 | RAS QDVNTAVA | 124 |
| | CDR2 | SASFLYS | 125 |
| | CDR3 | QQHYTTPPT | 126 |
| BACE001VL | CDR1 | RAS QDVS TAVA | 127 |
| | CDR2 | SASFLYS | 128 |
| | CDR3 | QQSYTTPPT | 129 |
| BACE002VL | CDR1 | RASQDVSTAVA | 127 |
| | CDR2 | SASFLYS | 128 |
| | CDR3 | QQFPTYLPT | 130 |
| BACE003VL | CDR1 | RASQDVSTAVA | 127 |
| | CDR2 | SASFLYS | 128 |
| | CDR3 | QQGYNDPPT | 131 |
| BACE004VL | CDR1 | RASQDVSTAVA | 127 |
| | CDR2 | SASFLYS | 128 |
| | CDR3 | QQSSTDPTT | 132 |
| BACE005VL | CDR1 | RAS QVVANS LA | 133 |
| | CDR2 | SASFLYS | 128 |
| | CDR3 | QQDATSPPTF | 134 |
| BACE006VL | CDR1 | RASQDVSTAVA | 127 |
| | CDR2 | SASFLYS | 128 |
| | CDR3 | QQYATDPPT | 135 |
| BACE001VH | CDR1 | GFTFSGYAIH | 136 |
| | CDR2 | GWISPAGGSTDYADSVKG | 137 |
| | CDR3 | GPFSPWVMDY | 138 |
| BACE002VH | CDR1 | GFTFLGYGIH | 139 |
| | CDR2 | GWISPAGGSTDYADSVKG | |
| | CDR3 | GPFSPWVMDY | 138 |
| BACE003VH | CDR1 | GFTFSGYAIH | 140 |
| | CDR2 | GWISPAGGSTDYADSVKG | 137 |
| | CDR3 | GPFSPWVMDY | 138 |
| BACE004VH | CDR1 | GFTFSGYAIH | 140 |
| | CDR2 | GWISPAGGSTDYADSVKG | 137 |
| | CDR3 | GPFSPWVMDY | 138 |
| BACE005VH | CDR1 | GFTFSGYAIH | 140 |
| | CDR2 | GWISPAGGSTDYADSVKG | 137 |
| | CDR3 | GPFSPWVMDY | 138 |
| BACE005VH | CDR1 | GFTFSGYAIH | 140 |
| | CDR2 | GWISPAGGSTDYADSVKG | 137 |
| | CDR3 | GPFSPWVMDY | 138 |
| ABETA001VH | CDR1 | GFTFSRYSMS | 141 |
| | CDR2 | QINSVGNSTYYPDTVKG | 142 |
| | CDR3 | GDY | 143 |
| ABETA001VL | CDR1 | RSSQSLIYSDGNAYLH | 144 |
| | CDR2 | KVSNRFS | 145 |
| | CDR3 | SQSTHVPWT | 146 |
| ABETA002VH | CDR1 | GFTFSSYAMS | 147 |
| | CDR2 | AINASGTRTYYA | 148 |
| | CDR3 | GKGNTHKPYGYVRYFDV | 149 |
| ABETA002VL | CDR1 | RASQSVSSSYLA | 150 |
| | CDR2 | GASSRAT | 151 |
| | CDR3 | LQIYNMPIT | 152 |
| VHH-TMEM30A | CDR1 | GFKITHYTMG | 153 |
| | CDR2 | RITWGGDNTFYSNSVKG | 154 |
| | CDR3 | GSTSTATPLRVDY | 155 |
| HIR-VH | CDR1 | GYTFTNYDIH | 156 |
| | CDR2 | WIYPGDGSTKYNEKFKG | 157 |
| | CDR3 | YWGQGTTV | 158 |
| HIR1-VL | CDR1 | RASQDIGGNLY | 159 |
| | CDR2 | ATSSLDS | 160 |
| | CDR3 | LQYSSSPWT | 161 |
| HIR2-VL | CDR1 | RASQDIGGNLY | 159 |
| | CDR2 | ATSSLDS | 160 |
| | CDR3 | LQYSSSPWT | 161 |
| HIR3-VL | CDR1 | RASQDIGGNLY | 159 |
| | CDR2 | ATSSLDS | 160 |
| | CDR3 | LQYSSSPWT | 161 |
| HIR4-VL | CDR1 | RASQDIGGNLY | 159 |
| | CDR2 | ATSSLDS | 160 |
| | CDR3 | LQYSSSPWT | 161 |
| 8C11-VH | CDR1 | DYNMN | 164 |
| | CDR2 | VINPNYGSSTYNQKFKG | 165 |
| | CDR3 | KWGQLGRGFFD | 166 |
| 8C11-VL | CDR1 | RASSSVSYMH | 167 |
| | CDR2 | ATSNLAS | 168 |
| | CDR3 | QQWSSSPLT | 169 |
| RGMA-VH | CDR1 | SHGIS | 172 |
| | CDR2 | WISPYSGNTNYAQKLQ | 173 |
| | CDR3 | VGSGPYYYMDV | 174 |
| RGMA-VL | CDR1 | TGTSSSVGDSIYVS | 175 |
| | CDR2 | DVTKRPS | 176 |
| | CDR3 | CSYAGTDTL | 177 |

TABLE 4
DVD Sequences

| SEQ | DVD | Outer | | Inner | |
|---|---|---|---|---|---|
| ID | Variable | Variable | | Variable | |
| NO | Domain | Domain | | Domain | |
| | Name | Name | Linker | Name | Sequence |
| | | | | | EVQLVESGGGLVQPGGSLRLSCAASGFTFS |
| | | | | | NYGMHWIRQAPGKGLEWIAMIYYDSSKMNY |
| | | | | | ADTVKGRFT I SRDNAKNSLYLQMNSLRAED |
| | | | | | TAVYYCAVPTSHYVVDVWGQGTTVTVS SAS |
| 40 | DVD2358H | AB402VH | HG-short | AB043VH | TKGPEVQLLESGGGLVQPGGSLRLSCAASG |
| | | | | | FTFSNYGMSWVRQAPGKGLEWVAS IRSGGG |
| | | | | | RTYYSDNVKGRFT I SRDNSKNTLYLQMNSL |
| | | | | | RAEDTAVYYCVRYDHYSGSSDYWGQGTLVT |
| | | | | | VSS |
| | | | | | AI QMTQSP SS LSASVGDRVT I TCQAS QD IG |
| | | | | | NWLAWYQQKPGKSPKLL I YGAT S LADGVPS |
| | | | | | RF SGSRSGTDFT LT I S SLQPEDFATYYCLQ |
| | | | | | AYNTPWTFGGGTKVE I KRTVAAPDVVMTQS |
| 41 | DVD2358L | AB402VL | LK-short | AB043VL | PLSLPVTPGEPAS I SCKSSQSLLDSDGKTY |
| | | | | | LNWLLQKPGQSPQRL I YLVSKL DSGVPDRF |
| | | | | | SGSGSGTDFTLKISRVEAEDVGVYYCWQGT |
| | | | | | HFPRTFGQGTKVE I KR |
| | | | | | EVQL LE SGGGLVQP GGSLRL S CAA SGFT F S |
| | | | | | NYGMSWVRQAPGKGLEWVAS I R SGGGRT YY |
| | | | | | SDNVKGRFT 1 S RDN SKNTLYLQMN SLRAE D |
| | | | | | TAVYYCVRYDHYSGSSDYWGQGTLVTVS SA |
| 42 | DVD2359H | AB43VH | HG-Short | AB4O2VH | SUMP EV+21D INES GGGINQPGGS LR LS CAAS |
| | | | | | GB-I F Stl YGYIRW I RQAPGKiGL. EW IAD11 1 YYDS |
| | | | | | SEMIYA D TVKiGRF T I SRDNAKI1 S LY141vIN S |
| | | | | | LRAEDTAVYYCAVP TSEYINDVWGOGTTVT |
| | | | | | VB 5 |
| | | | | | DVVMTQ SP LSLPVT PGEFAS I S CKSS QS LL |
| | | | | | DS DGKT Y LNWLL QKPGQS PQRL I Y LVS KL D |
| | | | | | SGVP DRF S GS GS GT DF TLK I S RVEAEDVGV |
| | | | | | YCWQGTHFFRTFGQGTKVE I KR TVAAPAI |
| 43 | DVD2359L | AB043VL | LK-short | AB402VL | QMTQSP SS LSASVGDRVT I TCQASQD I GNW |
| | | | | | LAWYOQ KPGKSP Kt, L, IYGAT S LADGvpsrd' |
| | | | | | SGS RS G T OFTLT I S SLOPEDFATYYCLOM |
| | | | | | NTPT4T F GGGT KVE I KR |
| | | | | | EVQLVESGGGLVQPGGSLRLSCAASGFTFS |
| | | | | | NYGMHWIRQAPGKGLEWIAMIYYDSSKMNY |
| | | | | | ADTVKGRFT I SRDNAKNSLYLQMNSLRAED |
| | | | | | TAVYYCAVPTSHYVVDVWGQGTTVTVS SAS |
| 44 | DVD2360H | AB403VH | HG-short | AB043VH | TKGPEVQLLESGGGLVQPGGSLRLSCAASG |
| | | | | | FTFSNYGMSWVRQAPGKGLEWVAS IRSGGG |
| | | | | | RTYYSDNVKGRFT I SRDNSKNTLYLQMNSL |
| | | | | | RAEDTAVYYCVRYDHYSGSSDYWGQGTLVT |
| | | | | | VS S |
| | | | | | EIVMTQSPATLSVSPGERATLSCQASQDIG |
| | | | | | NWLAWYQQKPGQSPRLL I YGAT S LADGVPA |
| | | | | | RF SGSRSGTEFT LT I S SLQSEDFAVYYCLQ |
| | | | | | AYNTPWTFGGGTKVE I KRTVAAPDVVMTQS |
| 45 | DVD2360L | AB403VL | LK-short | AB043VL | PLSLPVTPGEPAS I SCKSSQSLLDSDGKTY |
| | | | | | LNWLLQKPGQSPQRL I YLVSKL DSGVPDRF |
| | | | | | SGSGSGTDFTLKISRVEAEDVGVYYCWQGT |
| | | | | | HFPRTFGQGTKVE I KR |
| 46 | | | | | |
| 48 | | | | | |
| | DVD2362H | | | | |
| | AB404VH | | | | |
| | HG-short | | | | |
| | AB043VH | | | | |
| | | | | | QVQLVQSGAEVKKPGASVKVSCKASGFTFS |
| | | | | | NYGMHWIRQAPGQGLEWIAMIYYDSSKMNY |
| | | | | | ADTVKGRFT I TRDNS TNT LYMELS SLRSED |
| | | | | | TAVYYCAVPTSHYVVDVWGQGTTVTVS SAS |
| | | | | | TKGPEVQLLESGGGLVQPGGSLRLSCAASG |
| | | | | | FTFSNYGMSWVRQAPGKGLEWVAS IRSGGG |
| | | | | | RTYYSDNVKGRFT I SRDNSKNTLYLQMNSL |
| | | | | | RAEDTAVYYCVRYDHYSGSSDYWGQGTLVT |
| | | | | | VS S |
| 49 | | | | | |
| | DVD2362L | | | | |
| | AB404VL | | | | |
| | LK-short | | | | |
| | AB043VL | | | | |
| | | | | | AI QMTQSP SS LSASVGDRVT I TCQAS QD IG |
| | | | | | NWLAWYQQKPGKSPKLL I YGAT S LADGVPS |
| | | | | | RF SGSRSGTDFT LT I S SLQPEDFATYYCLQ |
| | | | | | AYNTPWTFGGGTKVE I KRTVAAPDVVMTQS |
| | | | | | PLSLPVTPGEPAS I SCKSSQSLLDSDGKTY |
| | | | | | LNWLLQKPGQSPQRL I YLVSKL DSGVPDRF |
| | | | | | SGSGSGTDFT LK I SRVEAEDVGVYYCWQGT |
| | | | | | HFPRTFGQGTKVE I KR |
| | | | | | Q |
| 52 | | | | | |
| | DVD2365H | | | | |
| | AB405VH | | | | |
| | HG-short | | | | |
| | AB043VH | | | | |
| | | | | | QVQLVQSGAEVKKPGASVKVSCKASGFTFS |
| | | | | | NYGMHWIRQAPGQGLEWIAMIYYDSSKMNY |
| | | | | | ADTVKGRFT I TRDNS TNT LYMELS SLRSED |
| | | | | | TAVYYCAVPTSHYVVDVWGQGT TV TVS SAS |
| | | | | | TKGPEVQLLESGGGLVQPGGSLRLSCAASG |
| | | | | | FTFSNYGMSWVRQAPGKGLEWVAS IRSGGG |
| | | | | | RTYYSDNVKGRFT I SRDNSKNTLYLQMNSL |
| | | | | | RAEDTAVYYCVRYDHYSGSSDYWGQGTLVT |
| | | | | | VS S |
| | | | | | EIVMTQSPATLSVSPGERATLSCQASQDIG |
| | | | | | NWLAWYQQKPGQSPRLL I YGAT S LADGVPA |
| | | | | | RF SGSRSGTEFT LT I S SLQSEDFAVYYCLQ |
| | | | | | AYNTPWTFGGGTKVE I KRTVAAPDVVMTQS |
| 53 | DVD2365L | AB405VL | | | LK-short AB043VL PLSLPVTPGEPAS I SCKSSQSLLDSDGKTY |
| | | | | | LNWLLQKPGQSPQRL I YLVSKL DSGVPDRF |
| | | | | | SGSGSGTDFTLKISRVEAEDVGVYYCWQGT |
| | | | | | HFPRTFGQGTKVE I KR |
| | | | | | EVQLVESGGGLVQPGNSLTLSCVASGFTFS |
| | | | | | NYGMHWIRQAPKKGLEWIAMIYYDSSKMNY |
| | | | | | ADTVKGRF T I SRDNSKNTLYLEMNSLRSED |
| | | | | | TAMYYCAVPT SHYVVDVWGQGVSVTVS SAS |
| 60 | DVD1294H | AB221 VH | | | HG-short AB043 VHTKGPEVQLLESGGGLVQPGGSLRLSCAASG |
| | | | | | FTFSNYGMSWVRQAPGKGLEWVAS IRSGGG |
| | | | | | RTYYSDNVKGRF T I SRDNSKNTLYLQMNSL |
| | | | | | RAEDTAVYYCVRYDHYSGSSDYWGQGTLVT |
| | | | | | VS S |
| | | | | | DI QMTQSPAS LSAS LEE IVT I TCQASQD IG |
| | | | | | NWLAWYQQKPGKSPQLL I YGAT SLADGVPS |
| | | | | | RF SGSRSGTQFS LK I SRVQVED IG IYYCLQ |
| | | | | | AB043 VLAYNTPWTFGGGTKLELKRTVAAPDVVMTQS |
| 61 | DVD1294L | AB221 VL | LK-short | | PLSLPVTPGEPAS I SCKSSQSLLDSDGKTY |
| | | | | | LNWLLQKPGQSPQRL I YLVSKL DSGVPDRF |
| | | | | | SGSGSGTDFTLKISRVEAEDVGVYYCWQGT |
| | | | | | HFPRTFGQGTKVE I KR |
| 62 | DVD2668H | AB221 VH | HG-long | AB043 VH | |
| same as | DVD2667H | | | | |
| 61 | DVD2668L | AB221 VL | LK-short | AB043 VL | |
| same as | DVD1294L | | | | |
| | | | | | EVQLLESGGGLVQPGGSLRLSCAASGFTFS |
| | | | | | NYGMSWVRQAPGKGLEWVASIRSGGGRTYY |
| | | | | | SDNVKGRFTISRDNSKNTLYLQMNSLRAED |
| | | | | | TAVYYCVRYDHYSGSSDYWGQGTLVTVSSA |
| 64 | DVD1295H | AB043 VH | HG-short | AB221 VH | STKGPEVQLVESGGGLVQPGNSLTLSCVAS |
| | | | | | GFTFSNYGMHWIRQAPKKGLEWIAMIYYDS |
| | | | | | SKMNYADTVKGRFTISRDNSKNTLYLEMNS |
| | | | | | LRSEDTAMYYCAVPTSHYVVDVWGQGVSVT |
| | | | | | VSS |
| | | | | | DVVMTQSPLSLPVTPGEPASISCKSSQSLL |
| | | | | | DSDGKTYLNWLLQKPGQSPQRLIYLVSKLD |
| | | | | | SGVPDRFSGSGSGTDFTLKISRVEAEDVGV |
| AB221 VL | | | | | YYCWQGTHFPRTFGQGTKVEIKRTVAAPDI |
| 65 | DVD1295L | AB043 VL | LK-short | | QMTQSPASLSASLEEIVTITCQASQDIGNW |
| | | | | | LAWYQQKPGKSPQLLIYGATSLADGVPSRF |
| | | | | | SGSRSGTQFSLKISRVQVEDIGIYYCLQAY |
| | | | | | NTPWTFGGGTKLELKR |
| | | | | | KM |
| Ial | | | | | |
| | | | | | EVQLVESGGGLVQPGGSLRLSCAASGFNIK |
| | | | | | DTYIHWVRQAPGKGLEWVARTYPTNGYTRY |
| | | | | | ADSVKGRFTISADTSKNTAYLQMNSLRAED |
| 68 | DVD2576H | ABOO4 VH | HG-short | VH | TAVYYCSRWGGDGFYAMDYWGQGTLVTVSS |
| | | | | | AS TKGPEVQLVESGGGLVQPGNSLTLSCVA |
| | | | | | SGFTFSNYGMHWIRQAPKKGLEWIAMIYYD |
| | | | | | SSKMNYADTVKGRFTISRDNSKNTLYLEMN |
| | | | | | SLRSEDTAMYYCAVPTSHYVVDVWGQGVSV |
| | | | | | TVS S |
| | | | | | DI QMTQSP SS LSASVGDRVT I TCRASQDVN |
| | | | | | TAVAWYQQKPGKAPKLL I YSASFLYSGVPS |
| | | | | | RF SGSRSGTDFT LT I S SLQPEDFATYYCQQ |
| DVD2576L | AB221 | | | | VLHYTTPPTFGQGTKVEIKRTVAAPDIQMTQS |
| 69 | ABOO4 VI_ | | LK-short | | PASLSASLEE IVT I TCQASQDIGNWLAWYQ |
| | | | | | QKPGKSPQLL IYGATSLADGVPSRFSGSRS |
| | | | | | GTQFSLKI SRVQVEDI GI YYCLQAYNTPWT |
| | | | | | FGGGTKLELKR |
| | | | | | KA |

### Example 2

### Design, Construction, and Analysis of a DVD-Ig™

1.1: Construction and Expression of Humanized Anti Mouse or Human Parent Antibodies

1.1.A: Selection of Human Antibody Frameworks

Each murine variable heavy and variable light chain gene sequence was separately aligned against 44 human immunoglobulin germline variable heavy chain or 46 germline variable light chain sequences (derived from NCBI Ig Blast website at http://www.ncbi.nlm.nih.gov/igblast/retrieveig.html.) using Vector NTI software. Humanization was based on amino acid sequence homology, CDR clusteranalysis, frequency of use among expressed human antibodies, and available information on the crystal structures of human antibodies. Taking into account possible effects on antibody binding, VH-VL pairing, and other factors, murine residues were mutated to human residues where murine and human framework residues are different, with a few exceptions. Additional humanization strategies were designed based on an analysis of human germline antibody sequences, or a subgroup thereof, that possessed a high degree of homology, i.e., sequence similarity, to the actual amino acid sequence of the murine antibody variable regions.

Homology modeling was used to identify residues unique to the murine antibody sequences that were predicted to be critical to the structure of the antibody combining site, the CDRs. Homology modeling is a computational method whereby approximate three dimensional coordinates were generated for a protein. The source of initial coordinates and guidance for their further refinement was a second protein, the reference protein, for which the three dimensional coordinates were known and the sequence of which was related to the sequence of the first protein. The relationship among the sequences of the two proteins is used to generate a correspondence between the reference protein and the protein for which coordinates are desired, the target protein. The primary sequences of the reference and target proteins are aligned with coordinates of identical portions of the two proteins transferred directly from the reference protein to the target protein. Coordinates for mismatched portions of the two proteins, e.g., from residue mutations, insertions, or deletions, are constructed from generic structural templates and energy refined to insure consistency with the already transferred model coordinates. This computational protein structure may be further refined or employed directly in modeling studies. The quality of the model structure is determined by the accuracy of the contention that the reference and target proteins are related and the precision with which the sequence alignment is constructed.

For the murine mAbs, a combination of BLAST searching and visual inspection is used to identify suitable reference structures. Sequence identity of 25% between the reference and target amino acid sequences is considered the minimum necessary to attempt a homology modeling exercise. Sequence alignments are constructed manually and model coordinates are generated with the program Jackal (see Petrey, D. et al. (2003) Proteins 53 (Suppl. 6): 430-435).

The primary sequences of the murine and human framework regions of the selected antibodies share significant identity. Residue positions that differ are candidates for inclusion of the murine residue in the humanized sequence in order to retain the observed binding potency of the murine antibody. A list of framework residues that differ between the human and murine sequences is constructed manually. Table 5 shows the framework sequences chosen for this study.

TABLE 5
Sequence Of Human IgG Heavy Chain Constant Domain And Light Chain
Constant Domain

| | SEQ | |
|---|---|---|
| Protein | ID NO | Sequence |
| Wild type hIgG1 | 70 | 1234567890123456789012345678901234567890 |
| constant region | | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| Mutant hIgG1 constant | 71 | ASTKGPSVFPLAPSSKSTSGGTAALGCLVKDYFPEPVTVS |
| region | | WNSGALTSGVHTFPAVLQSSGLYSLSSVVTVPSSSLGTQT |
| | | YICNVNHKPSNTKVDKKVEPKSCDKTHTCPPCPAPEAAGG |
| | | PSVFLFPPKPKDTLMISRTPEVTCVVVDVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| Ig kappa constant | 72 | TVAAPSVFIFPPSDEQLKSGTASVVCLLNNFYPREAKVQW |
| region | | KVDNALQSGNSQESVTEQDSKDSTYSLSSTLTLSKADYEK |
| | | HKVYACEVTHQGLSSPVTKSFNRGEC |
| Ig Lambda constant | 73 | QPKAAPSVTLFPPSSEELQANKATLVCLISDFYPGAVTVA |
| region | | WKADSSPVKAGVETTTPSKQSNNKYAASSYLSLTPEQWKS |
| | | HRSYSCQVTHEGSTVEKTVAPTECS |

The likelihood that a given framework residue would impact the binding properties of the antibody depends on its proximity to the CDR residues. Therefore, using the model structures, the residues that differ between the murine and human sequences are ranked according to their distance from any atom in the CDRs. Those residues that fell within 4.5 Å of any CDR atom are identified as most important and are recommended to be candidates for retention of the murine residue in the humanized antibody (i.e., back mutation).

### Example 3

### Assays Used to Identify and Characterize Parent Antibodies and DVD-Ig™

The following assays were used throughout the Examples to identify and characterize parent antibodies and DVD-Ig™, unless otherwise stated.

3.1 Size Exclusion Chromatography

Antibodies are diluted to 2.5 mg/mL with water and 20 mL is analyzed on a Shimadzu HPLC system using a TSK gel G3000 SWXL column (Tosoh Bioscience, cat# k5539-05k). Samples are eluted from the column with 211 mM sodium sulfate, 92 mM sodium phosphate, pH 7.0, at a flow rate of 0.3 mL/minutes. The HPLC system operating conditions are the following:

Mobile phase: 211 mM Na2SO4, 92 mM Na2HPO4*7H2O, pH 7.0

Gradient: Isocratic

Flow rate: 0.3 mL/minute

Detector wavelength: 280 nm

Autosampler cooler temp: 4° C.

Column oven temperature: Ambient

Run time: 50 minutes

Table 6 contains DVD-Ig™ constructs expressed as percent monomer (unaggregated protein of the expected molecular weight) as determined by the above protocol.

TABLE 6
Purity of DVD-Ig ™ Constructs as Determined by Size Exclusion
Chromatography

| | N-terminal/outer | C-terminal/inner | |
|---|---|---|---|
| | Variable Domain | Variable Domain | |
| DVD-Ig ID | (VD | (VD) | % Monomer (purity) |
| DVD1294 | TfR | APP | 95 |
| DVD2669 | TfR | APP | 100 |
| DVD2668 | TfR | APP | 98.1 |
| DVD2667 | TfR | APP | 98.4 |
| DVD1295 | APP | TfR | 97 |
| DVD2671 | APP | TfR | 96.5 |
| DVD2358 | TfR(AB402) | APP | 98 |
| DVD2359 | APP | TfR(AB402) | 96 |
| DVD2360 | TfR(AB403) | APP | 98 |
| DVD2361 | APP | TfR(AB403) | 95 |
| DVD2362 | TfR(AB404) | APP | 97.8 |
| DVD2363 | APP | TfR(AB404) | 96.8 |
| DVD2365 | TfR(AB405) | APP | 93 |
| DVD2366 | APP | TfR(AB405) | 100 |
| DVD2575 | TfR | Her2 | 100 |
| DVD2576 | Her2 | TfR | 97 |

DVD-Ig™s showed an excellent SEC profile with all DVD-Ig™ showing >90% monomer. This DVD-Ig™ profile is similar to that observed for parent antibodies.

### Example 4

### Assays Used to Determine Binding and Affinity of Parent Antibodies and DVD-Ig™ for their Target Antigen(s)

A: Cell-Based Electrochemiluminescence-Meso Scale Discovery Assay (ECL-MSD) Binding Assay

Meso-scale Discovery (MSD) Electrochemiluminescesnce (ECL) assays to screen for antibodies or DVDs that bind a desired target antigen expressed on the cell surface were performed as follows: Hek 293 cells overexpressing mouse Transferin Receptor were added onto MSD 96-well plates (MSD Cat# L11 XB-3, lot#2290-EA) which were blocked using blocking buffer (30% FBS Serum (Hyclone) in PBS) at 37° C. for 1 hr. After incubating at RT for 30 min with mild agitation, plates were washed with DPBS 3 times and Abs/DVDs (10,000 ng/ml) were added. After incubating for 1 hr at RT, plates were washed 3 times with DPBS and 25 ul of Goat anti human Sulfo TAG (MSD Cat# R32AC-, Lot# W001162) at 1 μg/ml is added. Following a 1 h incubation at RT for 1 hr, plates are washed and MSD read buffer is added before reading on MSD SECTOR Imager 6000. EC50 values are obtained using Xlfit4 software package

### Example 5

### Affinity Determination Using BIACORE Technology BIACORE Methods

The BIACORE assay (Biacore, Inc, Piscataway, N.J.) was used to determine the affinity of antibodies or DVD-Ig™ with kinetic measurements of on-rate and off-rate constants. Binding of antibodies or DVD-Ig™ to a target antigen (for example, a purified recombinant target antigen) was determined by surface plasmon resonance-based measurements with a Biacore® 1000 or 3000 instrument (Biacore® AB, Uppsala, Sweden) using running HBS-EP (10 mM HEPES [pH 7.4], 150 mM NaCl, 3 mM EDTA, and 0.005% surfactant P20) at 25° C. All chemicals were obtained from Biacore® AB (Uppsala, Sweden) or otherwise from a different source as described in the text. For example, approximately 5000 RU of goat anti-human IgG, (Fcγ), fragment specific polyclonal antibody (Pierce Biotechnology Inc, Rockford, Ill.) diluted in 10 mM sodium acetate (pH 4.5) was directly immobilized across a CM5 research grade biosensor chip using a standard amine coupling kit according to manufacturer's instructions and procedures at 25 μg/ml. Unreacted moieties on the biosensor surface are blocked with ethanolamine. Modified carboxymethyl dextran surface in flowcell 2 and 4 is used as a reaction surface. Unmodified carboxymethyl dextran without goat anti-human IgG in flow cell 1 and 3 is used as the reference surface. For kinetic analysis, rate equations derived from the 1:1 Langmuir binding model were fitted simultaneously to association and dissociation phases of all eight injections (using global fit analysis) with the use of Biaevaluation 4.0.1 software. Purified antibodies or DVD-Ig™ were diluted in HEPES-buffered saline for capture across goat anti-human IgG specific reaction surfaces. Antibodies or DVD-Ig™ to be captured as a ligand (25 μg/ml) were injected over reaction matrices at a flow rate of 5 μl/min. The association and dissociation rate constants, kon (M⁻¹s⁻¹) and koff (s⁻¹) are determined under a continuous flow rate of 25 μl/min. Rate constants are derived by making kinetic binding measurements at different antigen concentrations ranging from 10-200 nM. The equilibrium dissociation constant (M) of the reaction between antibodies or DVD-Ig™s and the target antigen is then calculated from the kinetic rate constants by the following formula: KD=koff/kon. Binding is recorded as a function of time and kinetic rate constants are calculated. In this assay, on-rates as fast as 106 M⁻¹s⁻¹ and off-rates as slow as 10⁻⁶s⁻¹ can be measured.

TABLE 7
Transferrin receptor binding assay and Biacorewith Transferrin
receptor Abs and DVD Igs

| | Expression | Hek293/mTfR | | | |
|---|---|---|---|---|---|
| Transferrin | yield, | MSD binding | Biacore, rmTfR (123-763) | | |
| receptor Abs | mg/L | assay, EC50 nM | Ka (1/Ms) | Kd (1/s) | KD (nM) |
| IgG | N/A | No Binding | | | |
| AB221 | 110 | 0.1 | 3.94E04* | 1.81E−04* | 5* |
| AB402 | 47.5 | 0.06 | 1.64E04 | 6.12E−05 | 3.7 |
| AB403 | 35.5 | 0.09 | 2.20E04 | 8.39E−05 | 3.8 |
| AB404 | 63 | 3.0 | | | No binding |
| AB405 | 34 | 13.6 | | | No Binding |
| DVD1294 | 11.7 | 0.11 | ND | ND | ND |
| DVD2669 | 8.8 | 0.12 | ND | ND | ND |
| DVD2668 | 9.7 | 0.10 | ND | ND | ND |
| DVD2667 | 9.3 | ND | ND | ND | ND |
| DVD1295 | 13.8 | 3.70 | ND | ND | ND |
| DVD2671 | 6.1 | 1.20 | ND | ND | ND |
| DVD2358 | 12.9 | 0.10 | 3.13E04 | 4.06E−05 | 1.3 |
| DVD2359 | 83.5 | 12.0 | slow | slow | LOD |
| DVD2360 | 50.4 | 0.15 | 2.80E04 | 4.80E−05 | 1.7 |
| DVD2361 | 62.8 | 10.7 | slow | slow | LOD |
| DVD2362 | 19.6 | 3.0 | Slow | Slow | LOD |
| DVD2363 | 21.8 | No binding | | | |
| DVD2365 | 13.8 | 3.2 | 9.87E03 | 7.71E−04 | 78 |
| DVD2366 | 21.8 | No binding | ND | ND | ND |
| DVD2575 | 62.8 | 0.11 | ND | ND | ND |
| DVD2576 | 80.2 | 3.40 | ND | ND | ND |

*measured using rmTfR (119-763)

Lower affinity variants of AB221, which are AB404 and 405, as determined by cell based MSD binding assay did not show significant binding by Biacore. DVD-Ig™s containing lower affinity variants of Ab221 showed reduced binding or no significant binding as measured by cell-based MSD binding assay or Biacore.

### Example 5

### In Vivo Biodistribution of Abs/DVDs

A: Measuring Antibody and DVD Ig™ Concentrations in Mouse Brain and Serum

Wildtype female C57B/6 mice ages 6 to 8 weeks were intravenously injected with anti-TfR variants, control IgG, anti-TfR containing DVD-Ig™s (20 mg/kg). After the indicated time, mice were perfused with D-PBS at a rate of 2 ml/min for 10 min. Brains were extracted and homogenized using Bullet Blender Blue (NextAdvance BBX24B) in 1% NP-40 (Calbiochem) in PBS containing CompleteMini EDTA-free protease inhibitor cocktail tablets (Roche Diagnostics). Homogenized brain samples were rotated at 4° C. for 1 hour before spinning at 14,000 rpm for 20 min. Supernatant was isolated for brain antibody measurement. Whole blood was collected before perfusion in serum separator microcontainer tubes (BDDiagnostics), allowed to clot for at least 30 min, and spun down at 5000 g for 90 s. Supernatant was isolated for antibody measurement in serum. Antibody or DVD-Ig™ concentrations in mouse serum and brain samples were measured with an ECL-MSD assay. MSD high bind 96-well plates (MSD cat # L11XB-3) were coated with a F(ab′)2 fragment of donkey anti-human IgG,Fc fragment-specific polyclonal antibody (Jackson ImmunoResearch) overnight at 4° C. Plates were blocked with 3% MSD Block buffer for 1 hour at 25° C. Each antibody or DVD-Ig™ was used as an internal standard to quantify respective antibody or DVD-Ig™ concentrations. Plates were washed with Wash Buffer and standards and samples diluted in 0.1% serum containing 1% MSD assay buffer were added and incubated for 2 hours at 25° C. Bound antibody was detected with Goat anti-human Sulfo-TAG, MSD and read on MSD SECTOR Imager 6000. Concentrations were determined from the standard curve with a five-parameter nonlinear regression program using Excel Fit software.

B. Immunohistochemistry

To determine antibody distribution in the brain, wild-type mice were intravenously injected with an indicated antibody or DVD (20 mg/kg or as indicated). After the indicated time, mice were perfused as described above, and brains were fixed in 4% paraformaldehyde for 8 hours. Following fixation, tissues were processed through a graded series of alcohol to xylene and then paraffin embedded. For histological evaluation, 5-μm brain sections were stained for the detection of anti-Human IgG.

First, the sections were de-paraffinized and rehydrated to water and placed into tris wash buffer. IHC staining was completed on a Dako autostainer links 48 system.

The sections were blocked with 3% hydrogen peroxide for 30 minutes, washed with wash buffer then incubated for 8 minutes with Protease. Sections were blocked with a streptavidin and biotin blocking kit (Vector Laboratories, Burlingame, Calif.) for 8 minutes each, followed by Dako protein block for 30 minutes. Next, the sections were incubated for 1 hour at room temperature with a biotinylated-Donkey anti-human IgG (F(ab′) fragmented antibody at 15 μg/ml followed by a streptavidin peroxidase reagent for 30 minutes. Following the streptavidin step the sections were reacted with DAB chromogen for 3 minutes to form a brown precipitate. The sections were then washed with water, counterstained, dehydrated and mounted for microscopic observation.

Image analysis: A Semi-quantitative analysis of mean parenchymal intensity per section was developed. The cerebellum and cortex sections were analyzed morphometrically using Image Pro Plus software. The analysis was performed on three images of parenchymal staining at a magnification of 20×. The average intensities of four representative Human IgG positive areas were selected per animal. All settings (filters and light levels) for each image were kept constant throughout the experiment. Measurements were analyzed as mean intensity measurements and exported to Microsoft Excel.

TABLE 8
In vivo biodistribution characteristics of transferrin receptor Abs with
20 mpk IV dosing

| Transferrin receptor | TfR | | Fold | | | Parenchymal | |
|---|---|---|---|---|---|---|---|
| Abs/in vivo | binding | | increase | Brain | Serum | staining | Neuronal staining |
| collection time/IV | EC50, | % ID/g | over | conc., | conc., | intensity | intensity |
| injection dose | nM | brain | IgG | nM | nM | Max = 4 | Max = 4 |
| IgG, 24 h, 20 mg/kg | NB | 0.3 +/− 0.03 | N/A | 1.14 +/− 0.17 | 1693 | 0 | 0 |
| AB221, 1 h, 20 mg/kg | 0.1 | 0.06 +/− 0.03 | | 2.14 +/− 0.13 | 2266 | 0 | 0 |
| AB221, 24 hr, 20 mg/kg | | 0.46 +/− 0.15 | 1.5 | 1.51 +/− 0.59 | 466 | 1.5 | 1.9 |
| AB221, 48 hr, 20 mg/kg | | 0.53 +/− 0.11 | | 1.73 +/− 0.43 | 46 | 2 | 2.3 |
| AB404, 1 h, 20 mg/kg | 3.0 | 0.84 +/− 0.28 | | 2.78 +/− 0.59 | 2580 | 0 | 0 |
| AB404, 24 hr, 20 mg/kg | | 1.97 +/− 0.11 | 6.6 | 6.48 +/− 1.09 | 1606 | 2.5 | 2.3 |
| AB404, 48 hr, 20 mg/kg | | 1.59 +/− 0.21 | | 4.83 +/− 0.27 | 993 | 2.5 | 1.5 |
| AB405, 1 h, 20 mg/kg | 13.6 | 1.05 +/− 0.15 | | 3.73 +/− 0.92 | 2566 | 0 | 0 |
| AB405, 24 hr, 20 mg/kg | | 1.97 +/− 0.33 | 6.6 | 6.97 +/− 0.44 | 1966 | 2 | 2 |
| AB405, 48 hr, 20 mg/kg | | 1.62 +/− 0.18 | | 5.96 +/− 0.35 | 933 | 2 | 2.3 |

Inverse relationship between brain uptake and affinity was observed with anti-TfR Abs listed on Table 8. Two lower affinity TfR Abs (AB404 and AB405) showed improved uptake, in some cases >8 fold increase in % injected dose over control IgG was measured. Twenty four hours after injection, strong parenchymal and neuronal staining was observed.

TABLE 9
In vivo biodistribution characteristics of transferrin receptor DVD-Ig ™ s at 24 hr
with 20 mg/kg IV injection (* 10 mpk injection)

| | | Fold increase | Brain | | Parenchymal/ |
|---|---|---|---|---|---|
| | | over control | conc., | Serum conc., | Neuronal |
| DVD-Ig ™ S ™ | % ID/g brain | DVD-Ig ™ | (nM) | (nM) | staining |
| Isotype control | 0.38 +/− 0.12 | N/A | 0.95 +/− 0.30 | 1420 | −/− |
| DVD-Ig ™ | | | | | |
| DVD2668 | 0.51 +/− 0.10 | 1.3 | 1.82 +/− 0.38 | 129.6 | −/− |
| DVD2667 | 3.87 +/− 0.85 | 10.2 | 4.79 +/− 0.41 | 855.1 | +/+ |
| DVD1295 | 1.87 +/− 0.19 | 5.0 | 7.74 +/− 0.64 | 763.3 | +/+ |
| DVD2671 | 0.97 +/− 0.26 | 2.6 | 2.90 +/− 0.44 | 588.2 | +/+ |
| DVD2359 | 0.84 +/− 0.23 | 2.2 | 3.04 +/− 0.34 | 1494.2 | +/+ |
| DVD2361 | 0.79 +/− 0.12 | 2.1 | 3.09 +/− 0.28 | 887.8 | +/+ |
| DVD2362 | 4.08 +/− 0.27 | 10.7 | 17.42 +/− 2.09 | 459.3 | +/+ |
| DVD2365 | 3.87 +/− 0.85 | 10.2 | 15.83 +/− 2.48 | 432.8 | +/+ |
| DVD2575 | 0.53 +/− 0.15 | 1.4 | 1.4 +/− 0.4 | 188.2 | +/+ |
| DVD2576 | 1.14 +/− 0.21 | 3.0 | 2.4 +/− 0.7 | 18.3 | +/+ |

Elevated DVD-Ig™s Levels Detected in Brain by Two Orthogonal Methods:

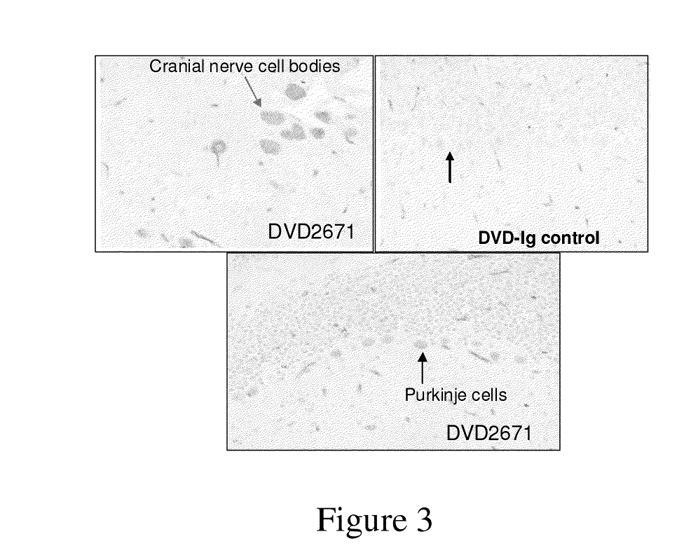

Localization to neuronal cells and parenchyma by IHC (FIG. 3 and Table 8) and higher than control DVD-Ig™ brain concentration as measured by MSD-ECL (Table 8) demonstrating transport across the BBB after therapeutic dosing via IV administration. For some of the DVD-Ig™s up to 10 fold higher brain uptake, which was measured by injected dose/gram of brain, was observed compare to control DVD-Ig™.

### Example 6

### Brain Uptake of a DVD-Ig™ Via Different Routes of Systemic Administration

Similar brain uptake observed with IV and IP administration (20 mg/kg) using DVD2671. SC administration yielded lower brain and serum concentrations in comparison.

TABLE 10
Brain uptake of 20 mpk DVD2671 after 24 h via different routes of
administration

| Route of administration | Brain conc., nM | Serum conc., nM |
|---|---|---|
| IV | 3.15 +/− 0.26 | 860 |
| IP | 3.72 +/− 0.82 | 730 |
| SC | 2.27 +/1.04 | 490 |

### Example 7

### Pharmacokinetic Study Using DVD-Ig™s

Mice were injected SC with indicated DVD-Ig™s at 20 mpk or 50 mpk and processed after 96 hours as described above. Another group was injected twice (at 0 and 48 hours) before processing after 96 hours as described. Brain serum concentration of 1.58+/−0.20 nM was retained at 96 h after single 20 mpk SC administration. Brain and serum concentrations measured after 24 h of IV injections of 20 mpk TfR Abs or DVD2671 from different studies are shown for comparison in Table 10.

TABLE 11
Brain and serum data for antibody binding proteins

| | | Time of sample | | |
|---|---|---|---|---|
| Ab/DVD | Treatment | collection (h) | Serum, (nM) | Brain, (nM) |
| DVD2671 | SC - single dose at | 1 | 3.7 +/− 3.1 | |
| | 0 h, 20 mpk | 24 | 142.5 +/− 3.2 | |
| | | 48 | 33.8 +/− 12.5 | |
| | | 72 | 8.8 +/− 2.5 | |
| | | 96 | 1.7 +/− 0.2 | 1.58 +/− 0.20 |
| | SC - single dose at | 1 | 11.25 +/− 7.5 | |
| | 0 h, 50 mpk | 24 | 680.0 +/− 143.6 | |
| | | 48 | 185.5 +/− 65.6 | |
| | | 72 | 93.8 +/− 36.4 | |
| | | 96 | 6.1 +/− 1.7 | 6.86 +/− 2.6 |
| | SC - multiple dose | 1 | 3.24 +/− 2.5 | |
| | at 0 and 48 h, | 24 | 110.0 +/− 16.83* | |
| | 40 mpk | 48 | 20.04 +/− 1.6 | |
| | | 72 | 365.0 +/− 189.12 | |
| | | 96 | 95.64 +/− 70.24 | 9.03 +/− 1.4 |
| IgG** | IV - single dose | 24 | 1791 +/− 127.3 | 1.5 +/− 0.3 |
| | 20 mpk | | | |
| AB221** | IV - single dose | 24 | 643.3 +/− 61.27 | 1.6 +/− 0.22 |
| | 20 mpk | | | |
| AB404* | IV - single dose | 24 | 1093 +/− 724 | 6.28 +/− 0.26 |
| | 20 mpk | | | |
| AB405* | IV - single dose | 24 | 1227 +/− 628.5 | 7.00 +/− 0.04 |
| | 20 mpk | | | |
| Control | IV - single dose | 24 | 1420 +/− 150 | 0.95 +/− 0.30 |
| DVD-Ig | 20 mpk | | | |
| DVD2671 | IV - single dose | 24 | 588.2 +/− 136.7 | 2.90 +/− 0.44 |
| | 20 mpk*** | | | |
| | IV - single dose | 48 | 118.75 +/− 62.90 | 3.07 +/− 0.28 |
| | 20 mpk | | | |
| | IV - single dose | 24 | 2396 +/− 929.4 | 5.08 +/− 1.25 |
| | 50 mpk* | | | |
| | IV - single dose | 48 | 1281.57 +/− 195 | 5.27 +/− 1.40 |
| | 50 mpk | | | |

n = 2
**n = 3
***n = 4

### Example 8

### DVD-Ig Generation and In Vitro/In Vivo Screening

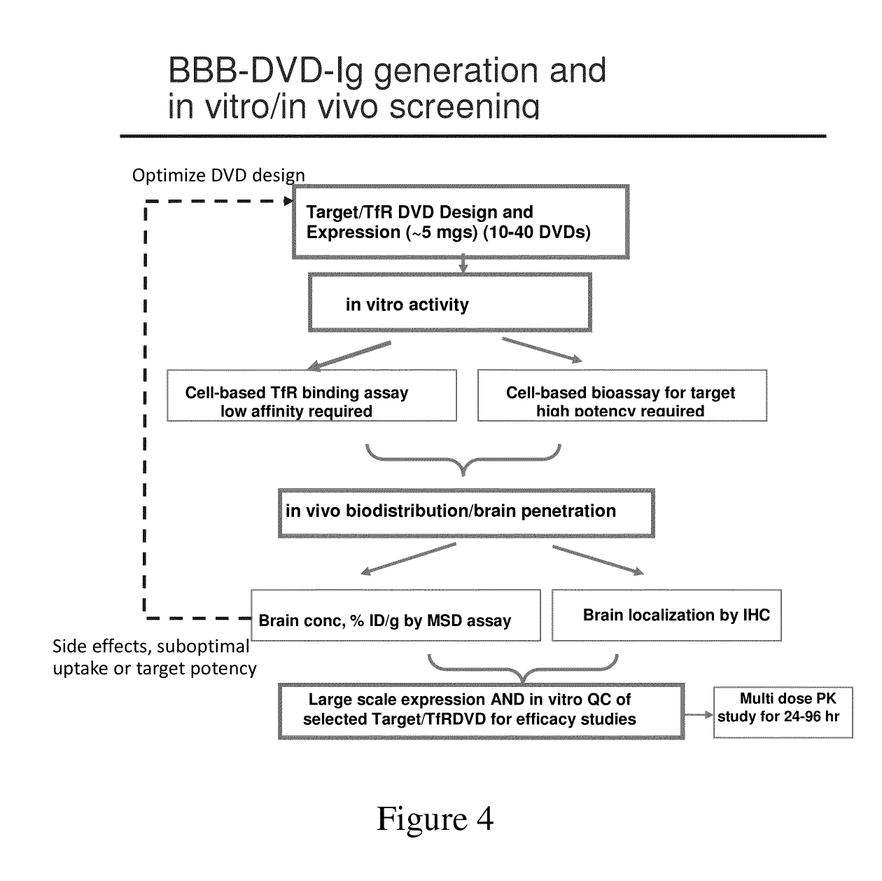

DVD-Ig binding proteins were generated using recombinant methods, and were screened using in vitro and in vivo systems described herein (FIG. 4). Methods and systems described in previous examples (e.g., assays Used to Identify and Characterize Parent Antibodies and DVD-Ig™) were used in the following examples unless indicated otherwise.

DVD-Ig binding proteins (approximately 10-40 DVDs) having domains that specifically bind a target and a TfR (target/TfR DVD) were designed and expressed at a concentration of about five milligrams (mg) using methods and material described herein. In vitro analysis was performed on the target/TfR DVD-Igs using a cell-based TfR binding assay (low affinity required) and a cell-based bioassay target (high potency required). The target/TfR DVD-Igs were then analyzed and compared in an in vivo biodistribution/brain penetration system using murine subjects. Specimens/samples (e.g., cells and tissues) from the subjects were analyzed to determine the presence of the target/TfR DVD-Ig in the subjects. A portion of the specimen/samples were analyzed to determine concentration of target/TfR DVD in the brain, and to calculate percent injected dose per gram tissue (% ID/g). Another portion of the specimens/samples were analyzed immunohistochemically to determine localization of the target/TfR DVD-Igs in the brain. Data for indicia of side effects, suboptimal uptake or target potency were analyzed, such that DVD-Ig™ design could be optimized by again designing and expressing target/TfR DVDs and analyzing using the assays and methods described above (e.g., in vitro activity and in vivo biodistribution/brain penetration).

The target/TfR DVD Igs having been tested and/or optimized were then expressed in a large scale. In vitro quality control (QC) methods and conditions were performed on the material used in efficacy studies. The resulting DVDs were then used in a multi-dose pharmacokinetics (PK) study over a period of 24-96 hours.

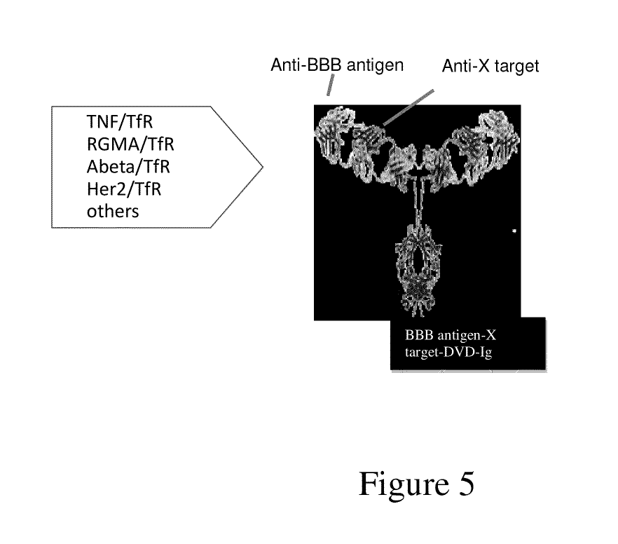

FIG. 5 shows an exemplary DVD-Ig binding protein. The DVD immunoglobulin includes at least one heavy chain variable domain that specifically binds a BBB antigen (anti-BBB antigen), and at least one heavy chain variable domain that specifically binds target X. Without being limited by any particular theory or mechanism of action, it is here envisioned that DVD-Igs engineered and analyzed by these methods efficiently penetrate the BBB and bind to target on or in the brain.

### Example 9

### In Vivo Tissue Distribution Analysis of DVD-Igs

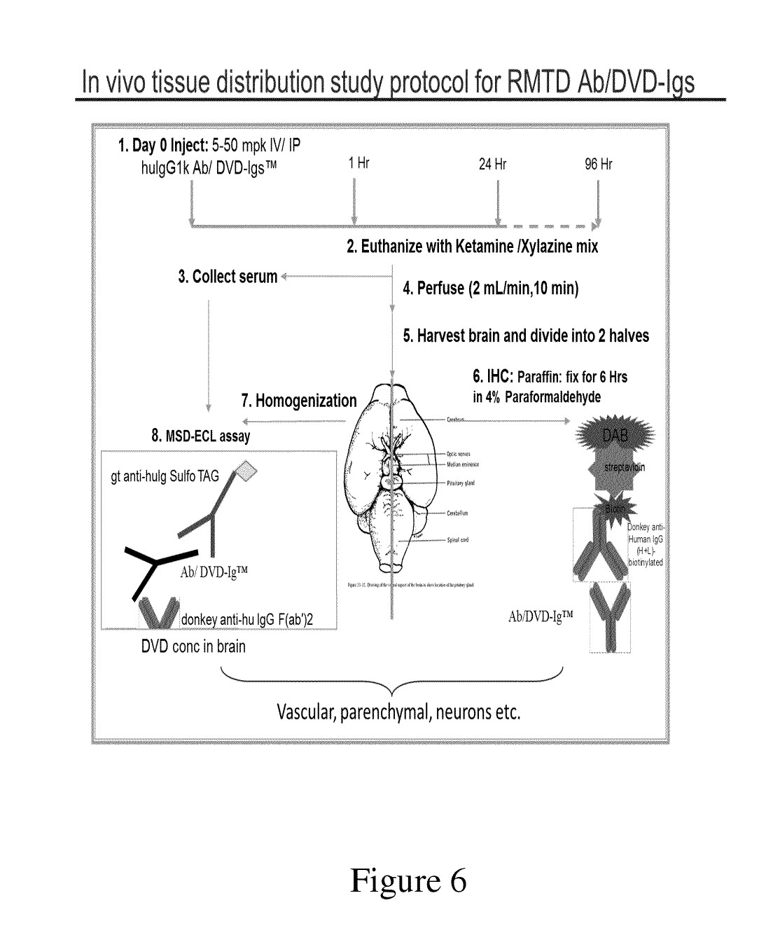

Analysis of in vivo tissue distribution for antibodies or a DVD-Igs was performed as shown in FIG. 6. At day 0, murine subjects were intravenously injected or intraperitoneally injected with 5-50 mg/kg (mpk) of a DVD-Ig™, or a control human IgG1 k antibody. The DVD-Ig™ contained a binding region specific for a BBB antigen and another binding region specific for a target. The subjects were injected at hour zero, one hour, twenty four hours or 96 hours. Subjects were sacrificed at the indicated time using a mixture of ketamine and xylazine.

Serum from each subject was collected and analyzed for concentration of control antibody or DVD-Ig™ using MSD-ESL assays. The assays used plates coated with F(ab′)2 fragment of donkey anti-human IgG which specifically bound both the human IgG1k antibody and a DVD Ig. Plates were then contacted with a full length anti-human Ig having a sulfo detection tag, and presence of antibody was detected.

After serum collection, subjects were perfused with D-PBS at a rate of 2 ml/min for 10 min. Brains were harvested and vertically sectioned/divided into equal halves (including equal portions of the cerebrum, optic nerves, pituitary gland, cerebellum and spinal cord). One half of the brain was homogenized and analyzed using an MSD-ECL assay. The other half of the brain was analyzed by immunohistochemical methods.

For the immunohistochemical analysis, tissues were treated with paraformaldehyde and then embedded in paraffin. The embedded material was stained for the detection of anti-Human IgG using a biotinylated donkey anti-human IgG (H+L). The material was then contacted with biotin, streptavidin and diaminobenzidine (DAB).

Data from the MSD-ECL assays were used to identify presence of the DVD-Igs in serum and in different tissues/cells of the brain, e.g., vascular, parenchymal and neuronal. Data for the DVD-Igs were compared to data obtained from subjects administered the control human IgG1 k antibody.

### Example 10

### Analysis of Anti-Mouse TfR(AB221) Antibody and Humanized Variants

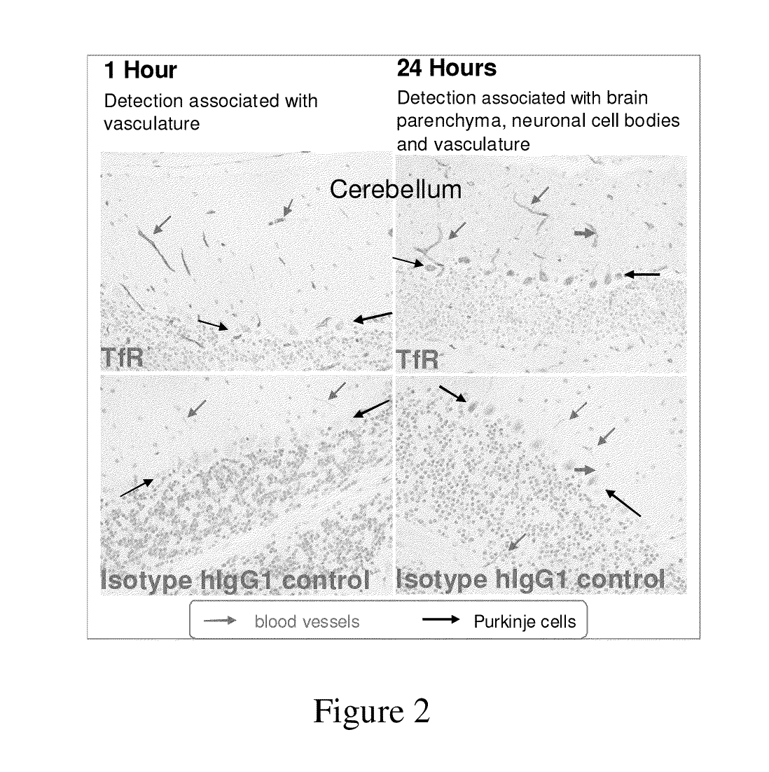

Examples herein analyzed the binding and BBB penetration characteristics of antibody AB221 (IgG2a antibody that specifically recognizes murine TfR) and humanized variants (Table 12). Presence of TfR antibody AB221 and its humanized variants were analyzed using assays and methods described in FIG. 6. Data show higher presence of TfR antibody AB221 and its humanized variants in the serum and brain compared to the human isotype IgG1 control. Immunohistochemical staining showed greater staining for AB221 anti-mouse TfR antibody in the blood vessels and Purkinje cells in the cerebellum compared to control isotype human IgG1 control (FIG. 2).

TABLE 12
TfR binding and in vivo biodistribution data for anti-mouse TfR(AB221)
antibody and humanized variants

| | | Brain conc. | | Fold | Serum | Parenchymal | Neuronal |
|---|---|---|---|---|---|---|---|
| | mTFR binding cell-based | (nM) | | increase | conc. | staining | staining |
| Name of Ab | assay (EC50, nM) | (20 mpk IV) | % ID/g brain | over IgG | (nM) | Max = 4 | Max = 4 |
| Control IgG | NB | 1.14 +/− 0.17 | 0.3 +/− 0.03 | N/A | | 0 | 0 |
| AB221 | 0.12 | 3.20 +/− 0.35 | 0.96 +/− 0.09 | 3.2 | 600.0 | 1.5 | 1.9 |
| AB402 | 0.06 | | | | | | |
| AB403 | 0.09 | | | | | | |
| AB404 | 3.0 | 6.0 +/− 0.50 | 1.5 | 5 | 580 | 2.5 | 2.3 |
| AB405 | 13.55 | 7.0 +/− 0.71 | 1.5 | 5 | 800 | 2 | 2 |

Without being limited by any particular theory or mechanism of action, it is here envisioned that lower affinity TfR antibodies more efficiently penetrate the BBB than higher affinity TfR antibodies on the outer position of the same DVD-Ig. Alternatively, it is envisioned also that a higher affinity TfR antibodies (as determined by binding assays or similar methods) on the inner position of a DVD-IG more efficiently penetrate the BBB than lower affinity TfR antibodies on the inner position of the same DVD-Ig.

### Example 11

### Elevated Levels of DVD-Igs Detected by Two Orthogonal Methods (IHC and MSD-ECL) and Ability to Transport Across the BBB after a Therapeutic Dosing

Orthogonal methods (MSD-ECL assays and IHC staining) were used in examples herein to determine whether DVD-Igs having a portion that binds TfR were effectively crossed the BBB and localized to the brain, e.g., neuronal cells and parenchyma. (Table 13).

TABLE 13
MSD-ECL assay data and immunohistochemical staining data for DVD-Igs

| | | | Fold increase | | |
|---|---|---|---|---|---|
| | IC50, | % Injected dose/g | over control | Parenchymal | Neuronal |
| DVD-Igs | nM | brain at 24 h | IgG | staining at 24 h | staining at 24 h |
| Isotype control DVD-Ig | NB | 0.013 ± 0.003 | N/A | No | No |
| TfRLSAbeta DVD2668 | 0.1 | 0.06 ± 0.01 | 4.6 | No | No |
| AbetaLSTfR DVD2671 | 1.2 | 0.14 ± 0.01 | 10.8 | Yes | Yes |
| TfRSSHer2 DVD2575 | 0.1 | 0.07 ± 0.01 | 5.4 | Yes | Yes |
| Her2SSTfR DVD2576 | 3.4 | 0.14 ± 0.01 | 10.8 | Yes | Yes |

The half maximal inhibitory concentration (IC₅₀) is a measure of the effectiveness of a compound in inhibiting biological or biochemical function. It was observed that the DVD-Igs containing a portion that specifically bound TfR more effectively inhibited TfR and were present at a higher concentration in the brain than control DVD-Ig™ (Table 13). The MSD-ECL and immunohistochemical data demonstrate that the DVD-Igs were effectively transported across the BBB after therapeutic dosing. FIG. 3 is an exemplary micrograph showing that the DVD-Igs were more localized in the parenchymal tissues and neuronal cells of the brain than subjects administered the control (non-TfR) DVD-Ig.

### Example 12

### Selection of TNF/TfR DVD-Igs

Table 14 contains a list of TNF/TfR DVD-Igs. The criteria for selection of DVDs for subsequent pain efficacy analysis included data showing low TfR binding affinity, as based on the data in Example 10 lower affinity TfR antibodies in the outer position of a DVD-Ig more efficiently penetrate the BBB. DVDs were further selected based on the highest concentration in the serum and brain, penetration through the BBB, and highest anti-TNF potency.

Data show that TNF antibody 8C11 strongly bound and inhibited TNF. The anti-TNF antibody 8C11 has the following binding VH and VL regions:

>VH
(SEQ ID NO: 162)
EFQLQQSGPELVKPGASVRISCKASGYSFTDYNMNWVKQSNGKSLEVVVG
VINPNYGSSTYNQKFKGKATLTVDQSSSTAYMQLNSLTSEDSAVYYCARK
WGQLGRGFFDVWGTGTTVTVSS
>VL
(SEQ ID NO: 163)
QIVLSQSPAILSASPGEKVTMTCRASSSVSYMHWFQQKPGSSPKPWIYAT
SNLASGVPARFSGSGSGTSYSLTISRVEAEDAATYYCQQWSSSPLTFGAG
TKLELKR

TABLE 14
TNF-TfR DVD-Igs analyzed for suitability for pain efficacy examples
551832 BBI-336PC 551832 BBI-336PC

| | | 293/ | | | | |
|---|---|---|---|---|---|---|
| | | mTFR cell- | | % ID/g brain | | |
| | % Monomer | based assay | BMP Assay | (10 mpk*/20 mpk**/ | | Seram Conc |
| Name of DVD pair | (parity) | (EC50, nM) | (EC50, nM) | 30 mpk***) | Brain conc. (nM) | (nM) |
| DVD control | 97.9 | NA | NA | 0.33 +/− 0.08*** | 2.5 +/− 0.63 | 2268 +/− 188.3 |
| RGMA (AE12-1) | 100% | NA | 0.25 | 0.4 +/− 0.16** | 5.9 +/− 2.1 | 4512 |
| RGMA(AE12-1)-LS-TfR(AB402) | 99.3 | 127.4 | 0.213 | 3.0 +/− 0.60** | 10.7 +/− 1.2 | 1183.75 |
| RGMA(AE12-1)-SL-TfR(AB402) | 90.5 | 2.13 | 0.367 | 1.7 +/− 0.34** | 6.5 +/− 0.89 | 818.75 |
| RGMA(AE12-1)-LL-TfR(AB402) | 91.8 | 1.67 | 1.78 | 1.4 +/− 0.21** | 6.2 +/− 0.89 | 533.75 |
| RGMA(AE12-1)-SS-TfR(AB402) | 96.4 | 30.85 | 0.436 | 3.9 +/− 0.62* | 7.8 +/− 0.95 | 669.5 |
| RGMA(AE12-1)-GS-TfR(AB402) | 91.4 | 5.36 | 0.014 | 6.3 +/− 0.75* | 9.9 +/− 1.2 | 187.04 |
| RGMA(AE12-1)-LS-TfR(AB403) | 99.1 | 25.73 | <0.025 nM | 10.1 +/− 1.5* | 18.4 +/− 5.0 | 481.4 |
| RGMA(AE12-1)-SS-TfR(AB403) | 98.2 | 71.03 | 1.18 | 4.4 +/− 0.49* | 9.3 +/− 1.11 | 486.9 |
| RGMA(AE12-1)-GS-TfR(AB403) | 98.8 | 4.3 | <0.025 nM | 3.1 +/− 0.36** | 14.8 +/− 0.65 | 396.6 |
| TfR(AB405)-SL-RGMA(AE12-1) | 95.2 | 4.41 | 0.73 | 2.4 +/− 0.42** | 13.1 +/− 2.8 | 642.8 |
| TfR(AB405)-LS-RGMA(AE12-1) | 99.2 | 3.9 | No inhibition | 11.4 +/− 3.99* | 18.3 +/− 1.7 | 462.2 |
| TfR(AB405)-GS-RGMA(AE12-1) | 90.1 | 28.07 | 3.12 | 8.9 +/− 0.48* | 19.1 +/− 1.88 | 611.9 |
| TfR(AB405)-LL-RGMA(AE12-1) | 98.9 | 4.44 | 0.701 | 10.2 +/− 0.48* | 19.0 +/− 2.91 | 754.35 |
| TfR(AB404)-LL-RGMA(AE12-1) | 94.1 | 3.61 | 13.25 | 11.9 +/− 2.63* | 24.8 +/− 3.77 | 464.4 |

Note that RGMA(AE12-1)-GS-TfR(AB403) and TfR(AB405)-SL-RGMA(AE12-1) were scaled up for efficacy.

Monoclonal antibody AB221 was used in examples herein and data using this antibody were compared to data using antibody 8C11. The six CDRs for antibody 8C11 (CDR-L1, -L2, and -L3 of light chain and CDR-H1, -H2, and -H3 of heavy chain are highlighted/bolded portions in SEQ ID NOs: 162-163 above. The CDRs of the heavy chain are DYNMN (SEQ ID NO: 164), VINPNYGSSTYNQKFKG (SEQ ID NO: 165), and KWGQLGRGFFD (SEQ ID NO: 166). The CDRs of the light chain are RASSSVSYMH (SEQ ID NO: 167), ATSNLAS (SEQ ID NO: 168), and QQWSSSPLT (SEQ ID NO: 169)

### Example 13

### Brain Concentration and Location Analysis of Selected DVD-Igs

DVD-Igs having a portion with antibody AB221 or humanized variant AB405 were constructed and analyzed in examples herein. Control DVD-Igs were also analyzed.

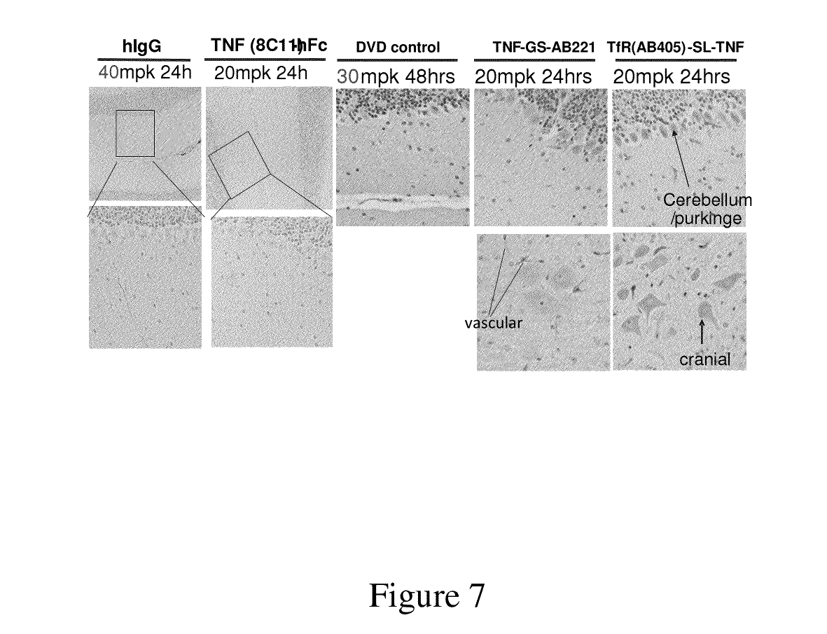

8C11-hFc DVD-Ig contained 8C11 antibody, which specifically binds TNF, and an antibody that binds the Fc portion of human Immunoglobulin G (hFc). The TNF-GS-AB221 DVD-Ig contained an antibody that binds TNF, a GS linker and monoclonal antibody AB221 (IgG2a) that specifically recognizes murine TfR. The TfR(AB405)-SL-TNF DVD-Ig contained AB405 (a humanized variant of AB221 antibody), a SL linker, and an antibody that binds TNF. Exemplary tissue staining data is shown in FIG. 7. Table 15 shows the concentration and localization data in the brain for subjects administered TfR(AB405)-SL-TNF DVD-Ig, TNF-GS-AB221 DVD, 8C11-hFc DVD-Ig, human IgG or the DVD control.

TABLE 15
DVD-Ig concentration and localization in the brain

| | | TNF | DVD | | |
|---|---|---|---|---|---|
| | | (8C11)- | control | TNF-GS- | TfR (AB405)-SL- |
| | HIgG (40 | hFc (20 | (30 mpk | AB221 (20 | TNF (20 mpk |
| | mpk 24 hr) | mpk 24 hr) | 48 hr) | mpk 24 hrs) | 24 hrs) |
| Brain (nM) | 5.4 +/− 1.0* | 3.1 +/− 1.8 | 2.5 +/− 0.63* | 5.58 +/− 0.88 | 16.2 +/− 1.3 |
| Spinal Cord (nM) | ND | ND | ND | ND | 10.0 +/− 0.5 |
| IHC Staining | 0/0.12 | 0/0.12 | 0/0 | 0.5/1 | 1.5/1.9 |
| max = 4 (Ave) | | | | | |
| Parenchyma/neuron | | | | | |

### Example 14

### Intrathecal Administration of TNF/TfR DVD-Igs Crossed the BBB and Effectively Reduced Pain

Examples herein analyzed the effectiveness of intrathecal administration of TNF/TfR DVD-Igs in a pain efficacy model. Partial nerve injuries, such as unilateral loose ligation or chronic constriction injury (CCI) of the sciatic nerve, result in the animal persistently holding the ipsilateral hindpaw in a guarded position. Depending on the tightness of ligation, the allodynia and hyperalgesia can persist for hours or days. The Bennett model as it is known involves a surgery to induce a nerve injury and is a well-known pharmacokinetics (PK) and pain efficacy model.

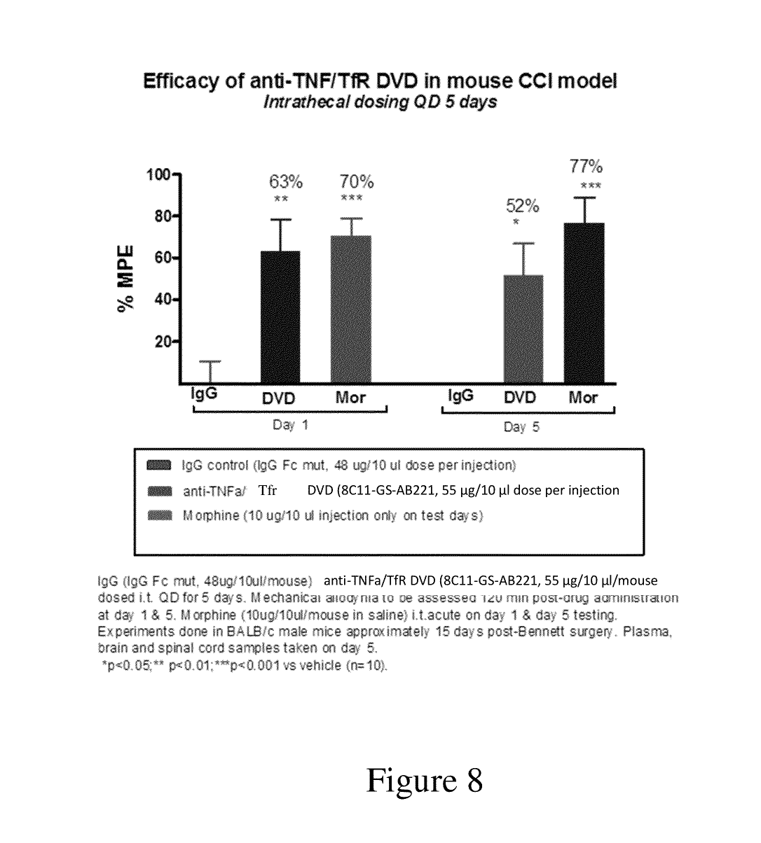

BALB/c murine subjects underwent a Bennett surgery and were intrathecally injected daily with either control IgG specific for mouse Fc (48 μg/10 μl dose per injection); 8C11-GS-AB221 DVD-Ig (anti-TNFa/anti-TfR; 55 μg/10 μl dose per injection); or morphine (10 μg/10 μl dose per injection). Subjects were injected daily. Mechanical allodynia was assessed in the above Bennett model 120 minutes post-injection administration at day 1 and day 5 (FIG. 8).

It was observed that intrathecal injection of the 8C11-GS-AB221 DVD-Ig (anti-TNFa/anti-TfR) in the Bennett model reduced more pain in subjects than intrathecal injection of the control IgG. Data for subjects intrathecally injected with the 8C11-GS-AB221 DVD-Ig were comparable to data observed for subjects intrathecally injected morphine. Data show that 17 nM of 8C11-GS-AB221 DVD-Ig was detected in the brain, and 52 nM of 8C11-GS-AB221 DVD-Ig was detected in the spinal cord. The amount of 8C11-GS-AB221 DVD-Ig present in the brain following an intravenous injection was observed to be similar (16 nM; Table 15), which shows tha efficiacious amounts were obtained in the brain by 20 mpK intravenous injection.

### Example 15

### Intravenous Administration of TNF/TfR DVD-Igs was Effective to Cross the BBB and Reduce Pain

Examples herein analyzed the effectiveness of intravenous administration of TNF/TfR DVD-Igs in the Bennett PK/pain efficacy model described above.

BALB/c murine subjects underwent a Bennett surgery and were intravenously injected with: control IgG specific for mouse Fc (48 μg/10 μl dose per injection); 8C11-GS-AB221 DVD (anti-TNFa/anti-TfR; 55 μg/10 μl dose per injection); or an acute post-operation dose of gabapentin (10 μg/10 μl dose per injection). Subjects were injected daily (20 mg/kg). Mechanical allodynia was assessed in the above Bennett model 120 minutes post-injection at day 1 and at day 5.

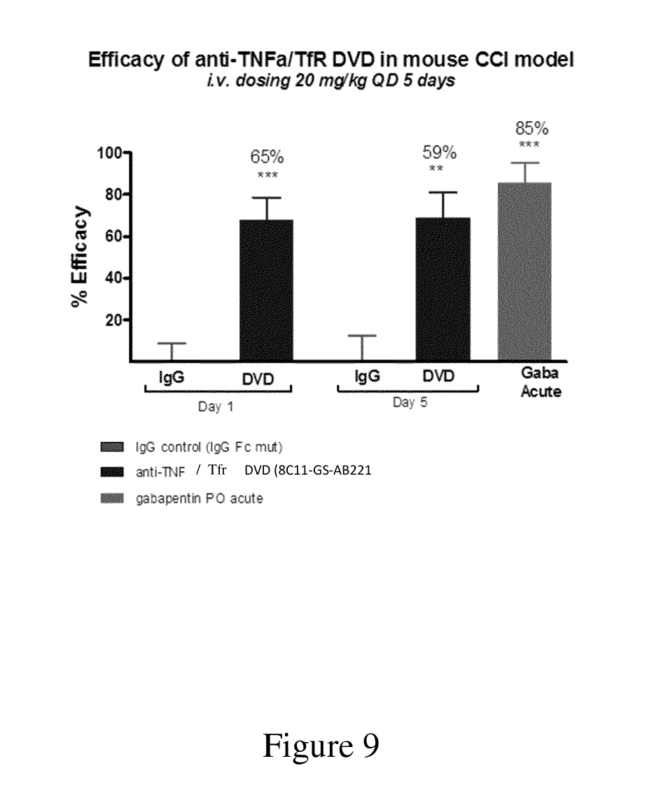

Data show that intravenous injection of the 8C11-GS-AB221 DVD-Ig (anti-TNFα/anti-TfR) reduced pain in subjects in the Bennett model better than intravenous injection of the control IgG (FIG. 9). Most importantly, data for intravenous injection of the 8C11-GS-AB221 DVD-Ig was only slightly less effective in obtaining pain reduction that intravenous injection of an acute dose of gabapentin. Data herein show that administration of 8C11-GS-AB221 DVD-Ig by either by an intrathecal administration or an intravenous administration was effective to reduce pain.

### Example 16

### RGMA-TfR DVD-Igs in a Multiple Sclerosis Model

The early stages of many neurodegenerative diseases are characterized by neurite damage and compromised synaptic function. Neurite degeneration often leads to neuronal cell death and impairs the conduction of signals in the affected nerves, causing impairment in sensation, movement, cognition, or other functions depending on which nerves are involved. Neurite degeneration is also a pathological indicator of the autoimmune disease multiple sclerosis (MS).

Repulsive guidance molecule A (RGMA) is a repulsive guidance molecule for retinal axons. After induced spinal cord injury RGMA accumulates in the scar tissue around the lesion, and presence of RGMA may be an inhibitor of axonal outgrowth.

Table 14 lists the RGMA-Tfr DVD-Igs that were analyzed for suitability for a model for MS. The DVD-IGs were engineered to have the anti-BBB antigen portion or anti-target portion in the outer position (N-terminus) or the inner position (C-terminus). Examples herein show that depending on the position of the anti-BBB epithelium antigen portion, one would either select a lower affinity antibody (outer position) or a higher affinity antibody (inner position). As this Example was using an anti-BBB antigen antibody in the outer position, the criteria for selection of DVD-Igs for use in the MS efficacy model was low TfR binding affinity, the highest anti-RGMA potency, the highest serum and brain concentrations, and greatest BBB penetration. Accordingly, DVD-Ig TfR(AB405)-SL-RGMA(AE12-1) and DVD-Ig TfR(AB405)-LS-RGMA(AE12-1) were selected, expressed in a larger scale and analyzed for efficacy in a MS model described herein. Alternatively, using an anti-BBB antigen antibody in the inner position one might have selected a DVD-Ig with higher TfR binding affinity, the highest anti-RGMA potency, the highest serum and brain concentrations, and greatest BBB penetration.

RGMA-JL3-VH (AE12-1) is
(SEQ ID NO: 170)
EVQLVQSGAEVKKPGASVKVSCKASGYTFTSHGISWVRQAPGQGLDWMG
WISPYSGNTNYAQKLQGRVTMTTDTSTSTAYMELSSLRSEDTAVYYCAR
VGSGPYYYMDVWGQGTLVTVSS
RGMA-JL3-VL (AE12-1)) is
(SEQ ID NO: 171)
QSALTQPRSVSGSPGQSVTISCTGTSSSVGDSIYVSWYQQHPGKAPKLM
KLYDVTRPSGVPDRFSGSKSGNTASLTISGLQAEDEADYYCCSYAGTDT
LFGGGTKVTVLG

The six CDRs (CDR-L1, -L2, and -L3 of light chain and CDR-H1, -H2, and -H3 of heavy chain are highlighted/bolded portions in SEQ ID NOs: 170-171 above. The CDRs of the heavy chain are SHGIS (SEQ ID NO: 172), WISPYSGNTNYAQKLQ (SEQ ID NO: 173), and VGSGPYYYMDV (SEQ ID NO: 174). The CDRs of the light chain are TGTSSSVGDSIYVS (SEQ ID NO: 175), DVTKRPS (SEQ ID NO: 176), and CSYAGTDTL (SEQ ID NO: 177). See U.S. patent publication 2010/0074900 published Mar. 25, 2010 and international publication WO/2013/112922 published Aug. 1, 2013, each of which is incorporated by reference herein in its entirety.

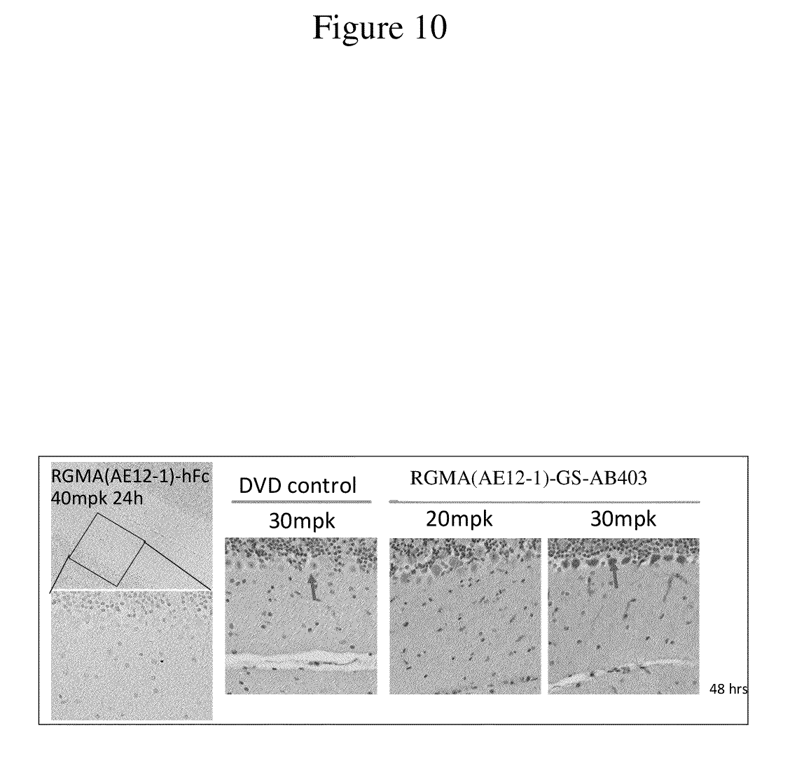

FIG. 10 is an exemplary micrograph of stained brain tissue from subjects administered either: 40 mpk of RGMA (AE12-1)-hFc at 24 hours; 30 mpk human IgG control; 20 mpk of RGMA (AE12-1)-GS-AB403 DVD-Ig, or 30 mpk of RGMA (AE12-1)-GS-AB403 DVD-Ig. Data show effective staining of brain tissues from subjects contacted with RGMA (AE12-1)-GS-AB403 DVD-Ig, indicating that the DVD-Ig effectively crossed the BBB and bound to RGMA on neuronal cells/tissue in the brain.

### Example 17

### Preparation and Analysis of Amyloid-Beta/TfR DVD-Igs

Bapineuzumab is a humanized monoclonal antibody (Mab) that targets the neurotoxic amyloid-beta peptide. Amyloid-beta (An) is an early biomarker of Alzheimer's disease and other neurodegenerative pathologies. Murine antibody 3D6 is the parent of the humanized monoclonal antibody Bapineuzumab,

DVD-Igs were designed and constructed that contained a portion that specifically bound to Aβ, and another portion that bound TfR. Examples of DVD Igs and components that bind Aβ are shown in Tables 2-4. Table 16 shows a list of Aβ/TfR DVD-Igs that were engineered and analyzed in assays and a model system for: binding to TfR and Aβ, for concentration in serum and the brain, and for IHC staining in the parenchyma and neurons.

Eight week old C57BI/6N female murine subjects were intravenously administered 20 mpk of the DVD-Igs. It was observed that the AB405-SL-Aβ(3D6) DVD-Ig, Aβ(3D6)-GS-AB403 DVD-Ig, and Aβ(3D6)-SS-AB402 DVD-Ig 2359 each had high potency again TfR and were present/localized in greater amount in brain tissue than subjects administered the control IgG.

TABLE 16
Aβ/TfR DVD-Igs DVD-Ig concentrations and localization in the brain

| | 293/mTFR | | | | | | | |
|---|---|---|---|---|---|---|---|---|
| | cell-based | | | | | | IHC (max = 4) staining | |
| | assay | Aβ binding | Serum (Terminal) | | Brain | | Parenchyma | Neuron |
| Ab/DVD | EC50, nM | (ng/mL) | μg/mL | nM | ng/mL | nM | (Ave) | (Ave) |
| AB405-SL-Aβ(3D6) | 4.71 | 73.9 | 84.2 ± 8.3 | 421.4 ± 41.6 | 2089 ± 269.4 | 10.4 ± 1.34 | 1.75 | 2.0 |
| Aβ(3D6)-GS-AB403 | 1.98 | 29.6 | 89.6 ± 10.9 | 448.1 ± 54.4 | 946.1 ± 84.9 | 4.7 ± 0.42 | 1.38 | 1.5 |
| Aβ(3D6)-SS-AB402 DVD2359 | 12 | ND | 259.8 ± 72.5 | 1299 ± 362.9 | 1332 ± 343.7 | 6.6 ± 1.71 | 1 | 1 |
| Aβ(3D6)-SS-AB405 DVD2366 | No Binding | ND | 248.6 ± 41.1 | 1243 ± 205.8 | 215.7 ± 22.9 | 1.0 ± 0.11 | 1 | 0.5 |
| Ig Fc Mut | No Binding | No binding | 379.1 ± 7.7 | 2527 ± 51.6 | 387.1 ± 102.6 | 2.5 ± 0.68 | 0.17 | 0 |

### Example 18

### TNF/TfR DVD-Igs Concentration and Localization in the Serum and Brain

Examples herein analyzed the concentration and localization of TfR(AB405)-SL-TNF DVD-Igs administered to subjects in a PK study described above. Subjects were administered different doses (single or multiple doses of 20-40 mpk) of TfR(AB405)-SL-TNF DVD-Igs either subcutaneously (SC), intravenously (IV), or intraperitoneally (IP).

Data in Table 17 show comparable serum and brain concentration for TfR(AB405)-SL-TNF DVD-Igs using either intravenous and intraperitoneal routes of administration.

TABLE 17
TfR(AB405)-SL-TNF DVD-Ig concentrations and localization in the brain

| | | | | | | IHC Staining | |
|---|---|---|---|---|---|---|---|
| | Time of | Serum | | Brain | Spinal cord | Max = 4 | |
| Treatment | collection (hr) | (ug/mL) | (nM) | (nM) | (nM) | (ave) | |
| SC - single dose | 1 | 0.63 +/− 0.25 | 3.1 +/− 1.2 | | | | |
| 20 mpk | 24 | 38.9 +/− 11.5 | 194.9 +/− 57.5 | 8.4 +/− 1.6 | 5.7 +/− 1.5 | 0.87 | 1.0 |
| IP - single dose | 1 | 34.7 +/− 8.6 | 173.6 +/− 43.2 | | | | |
| 20 mpk | 24 | 109.4 +/− 16.2 | 547.2 +/− 81.3 | 16.2 +/− 1.3 | 10.0 +/− 0.5 | 1.5 | 1.9 |
| IV - single dose | 1 | 83.8 +/− 21.1 | 419.5 +/− 105.9 | | | | |
| 20 mpk | 24 | 109.0 +/− 20.9 | 545.0 +/− 104.7 | 16.8 +/− 1.8 | 9.3 +/− 1.5 | 1.9 | 1.4 |
| SC - single dose | 1 | 1.0 +/− 0.69 | 5.4 +/− 3.4 | | | | |
| 40 mpk | 24 | 88.0 +/− 7.3 | 440.0 +/− 36.9 | 16.2 +/− 1.7 | 8.0 +/− 1.3 | 1.1 | 0.87 |
| IP - single dose | 1 | 45.0 +/− 11.5 | 225.5 +/− 57.8 | | | | |
| 40 mpk | 24 | 218.5 +/− 15.1 | 109.2 +/− 75.4 | 22.4 +/− 4.3 | 9.6 +/− 2.2 | 1.5 | 0.87 |
| IV - single dose | 1 | 168.7 +/− 44.6 | 843.3 +/− 223.4 | | | | |
| 40 mpk | 24 | 172.6 +/− 9.9 | 863.2 +/− 49.9 | 20.7 +/− 2.6 | 10.2 +/− 2.6 | 2.2 | 1.7 |
| IV - multiple dose | 48 | 860.4 +/− 65.3 | 4302 +/− 326.9 | 23.8 +/− 2.7 | | | |
| 20 mpk | | | | | | | |

### Example 19

### TfR/RGMA DVD-Ig Concentration and Localization in the Serum and Brain

Examples herein analyzed the concentration and localization of AB405-SL-RGMA DVD-Igs administered to male BALB/c murine subjects in a PK study described above. Subjects were administered AB405-SL-RGMA DVD-Igs at different doses (single or multiple doses of 20-40 mpk) either subcutaneously (SC), intravenously (IV), or intraperitoneally (IP). Table 18 shows concentration and localization data in serum and the brain for subjects administered AB405-SL-RGMA DVD-Igs.

TABLE 18
AB405-SL-RGMA DVD-Igs concentration and localization in serum and the
brain in a PK study

| | | Time of | | | | | | |
|---|---|---|---|---|---|---|---|---|
| | | tissue | | | | | IHC Staining | |
| | | collection | Serum | | Brain | Spinal Cord | max = 4 | |
| Ab/DVD | Treatment | (h) | ug/mL | (nM) | (nM) | (nM) | (Ave) | |
| AB405-SL- | IV - single | 1 | 115.4 +/− 25.5 | 576.9 +/− 127.8 | | | | |
| RGMA | dose 20 mpk | 24 | 98.6 +/− 7.7 | 601.8 +/− 419.0 | 21.6 +/− 4.6 | 8.3 +/− 3.9 | 0.75 | 0.75 |
| | | 48 | 40.9 +/− 6.1 | 204.9 +/− 30.7 | 17.9 +/− 1.6 | 8.2 +/− 1.5 | 1.1 | 1.1 |
| | IV - single | 1 | 289.3 +/− 33.2 | 1446 +/− 166.6 | | | | |
| | dose 40 mpk | 24 | 207.1 +/− 21.7 | 1036 +/− 108.7 | 25.7 +/− 4.0 | 13.4 +/− 1.8 | 1.5 | 0.87 |
| | | 48 | 79.7 +/− 15.8 | 398.5 +/− 79.4 | 23.3 +/− 2.1 | 11.43 +/− 1.2 | 1.7 | 0.87 |
| | IV - multiple | 48 | 538.9 +/− 26.9 | 2273 +/− 848.8 | 30.4 +/− 4.6 | ND | | |
| | dose 20 mpk | | | | | | | |
| | @ 0, 24, 48 hr | | | | | | | |
| RGMA hFc | IV - single | 24 | 653.0 +/− 151.6 | 4354 +/− 1011 | 5.3 +/− 1.8 | ND | 0.25 | 0 |
| Lot 1904318 | dose 40 mpk | | | | | | | |
| IgG Fc Mut | IV - single | 24 | 449.8 +/− 77.3 | 2999 +/− 515.6 | 5.4 +/− 1.0 | ND | 0 | 0.12 |
| Lot 1804646 | dose 40 mpk | | | | | | | |

Without being limited by any particular theory or mechanism of action, it is here envisioned that the DVD-Igs described herein in Examples were detected by IHC staining in parenchyma and neuronal cells in two hours following a single systemic injection (20 mpk, intravenous). Increased brain uptake was observed at 24 hours after injection. Further, the DVD-Igs were retained in the brain for at least about 96 hours following a single intravenous injection of 50 mpk. The DVD-Igs accumulated in the brain by using multiple injections, for example using two 20 mpk intravenous injections. In fact, similar brain uptake data was observed with either intravenous.

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Ausubel et al. (eds.), CURRENT PROTOCOLS IN MOLECULAR BIOLOGY, John Wiley &Sons, NY (1993);
Ausubel, F. M. et al. eds., SHORT PROTOCOLS IN MOLECULAR BIOLOGY (4th Ed. 1999) John Wiley & Sons, NY. (ISBN 0-471-32938-X);
Bergman I, Burckart G J, Pohl C R, Venkataramanan R, Barmada M A, Griffin J A, Cheung. Pharmacokinetics of IgG and IgM anti-ganglioside antibodies in rats and monkeys after intrathecal administration. J Pharmacol Exp Ther. 1998 January; 284(1):111-5;
Braen A P, Perron J, Tellier P, Catala A R, Kolaitis G, Geng W. A 4-week intrathecal toxicity and pharmacokinetic study with trastuzumab in cynomolgus monkeys. Int J Toxicol. 2010 May-June; 29(3):259-67;
CONTROLLED DRUG BIOAVAILABILITY, DRUG PRODUCT DESIGN AND PERFORMANCE, Smolen and Ball (eds.), Wiley, New York (1984);
Garg A, Balthasar JP. Investigation of the influence of FcRn on the distribution of IgG to the brain. AAPS J. 2009 September; 11(3):553-7;
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, N.Y., (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, U.S. Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).
Kriegler, Gene Transfer and Expression, A Laboratory Manual, Stockton Press, NY (1990); Levites Y, Smithson L A, Price R W, Dakin R S, Yuan B, Sierks M R, Kim J, McGowan E, Reed D K, Rosenberry T L, Das P, Golde T E. Insights into the mechanisms of action of anti-Abeta antibodies in Alzheimer's disease mouse models. FASEB J. 2006 December; 20(14):2576-8. Epub 2006 Oct. 26;
Lu and Weiner eds., CLONING AND EXPRESSION VECTORS FOR GENE FUNCTION ANALYSIS (2001) BioTechniques Press. Westborough, Mass. 298 pp. (ISBN 1-881299-21-X). MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION To GENETIC ENGINEERING (3d Ed. 1985) Blackwell Scientific Publications, Boston. Studies in Microbiology; V. 2:409 pp. (ISBN 0-632-01318-4).
Sambrook, J. et al. eds., MOLECULAR CLONING: A LABORATORY MANUAL (2d Ed. 1989) Cold Spring Harbor Laboratory Press, NY. Vols. 1-3. (ISBN 0-87969-309-6).
Shen D D, Artru A A, Adkison K K. Principles and applicability of CSF sampling for the assessment of CNS drug delivery and pharmacodynamics. Adv Drug Deliv Rev. 2004 Oct. 14; 56(12):1 825-57.
SUSTAINED AND CONTROLLED RELEASE DRUG DELIVERY SYSTEMS, J. R. Robinson, ed., Marcel Dekker, Inc., New York, 1978
Winnacker, E. L. FROM GENES To CLONES: INTRODUCTION To GENE TECHNOLOGY (1987) VCH Publishers, NY (translated by Horst lbelgaufts). 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A dual variable domain (DVD) binding protein that specifically binds to an antigen expressed on brain vascular epithelium of a subject and facilitates uptake of a composition into the brain of the subject.

2-4. (canceled)

5. The binding protein of claim 1, wherein the target is transferrin receptor.

6-12. (canceled)

13. The binding protein claim 1, further comprising a composition wherein the composition is co-administered with the binding protein.

14-16. (canceled)

17. The binding protein of claim 1 comprising a polypeptide chain, wherein the polypeptide chain comprises VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1; and
wherein the binding protein specifically binds TfR or HIR; and
(a) VD1 or VD2 comprises three CDRs wherein at least one CDR comprises an amino acid sequence selected from the group consisting of SEQ ID NO: 76, 77, 78, 82, 83, 115-117 and 156-158;
(b) VD1 and VD2 independently comprise three CDRs wherein at least one CDR comprises an amino acid sequence selected from the group consisting of SEQ ID NO: 76, 77, 78, 82, 83, 115-117 and 156-158; or
(c) VD1 comprises three CDRs wherein at least one CDR comprises an amino acid sequence selected from the group consisting of SEQ ID NO: 76, 77, 78, 82, 83, 115-117 and 156-158, and VD2 comprises three CDRs wherein at least one CDR comprises an amino acid sequence selected from the group consisting of SEQ ID NO: 76, 77, 78, 82, 83, 115-117 and 156-158.

18. The binding protein of claim 17, wherein
(a) VD1 or VD2 comprises an amino acid sequence selected from the group consisting of SEQ ID NO: 30, 32, 34, 36, 56, and 104; or
(b) VD1 and VD2 independently comprise an amino acid sequence selected from the group consisting of SEQ ID NO: 30, 32, 34, 36, 56, and 104

19. The binding protein of claim 1 comprising a polypeptide chain, wherein the polypeptide chain comprises VD1-(X1)n-VD2-C-(X2)n, wherein
VD1 is a first light chain variable domain;
VD2 is a second light chain variable domain;
C is a light chain constant domain;
X1 is a linker with the proviso that it is not CL;
X2 does not comprise an Fc region;
(X1)n is (X1)0 or (X1)1;
(X2)n is (X2)0 or (X2)1; and
wherein the binding protein specifically binds TfR or HIR; and
(a) VD1 or VD2 comprises three CDRs each, wherein at least one CDR comprises an amino acid sequence selected from the group consisting of SEQ ID NO: 79, 80, 81, 84, 85, 86, 118, 119, 120, 159, 160, and 161;
(b) VD1 and VD2 independently comprise three CDRs each comprising amino acid sequences selected from the group consisting of SEQ ID NO: 79, 80, 81, 84, 85, 86, 118, 119, 120, 159, 160, and 161; or
(c) VD1 comprises three CDRs each comprising amino acid sequences selected from the group consisting of SEQ ID NO: 79, 80, 81, 84, 85, 86, 118, 119, 120, 159, 160, and 161, and VD2 comprises three CDRs each comprising amino acid sequences selected from the group consisting of SEQ ID NO: 79, 80, 81, 84, 85, 86, 118, 119, 120, 159, 160, and 161.

20. The binding protein of claim 19, wherein
(a) VD1 or VD2 comprises an amino acid sequence selected from the group consisting of SEQ ID NO: 31, 33, 35, 37, 57, 105, 106, 107, and 108; or
(b) VD1 and VD2 independently comprise an amino acid sequence selected from the group consisting of SEQ ID NO: 31, 33, 35, 37, 57, 105, 106, 107, and 108.

21. The binding protein of claim 19, wherein (X1)n is (X1)0.

22. The binding protein of claim 1 comprising first and second polypeptide chains, wherein the first polypeptide chain comprises a first VD1-(X1)n-VD2-C-(X2)n, wherein
VD 1 is a first heavy chain variable domain;
VD2 is a second heavy chain variable domain;
C is a heavy chain constant domain;
X1 is a first linker;
X2 is an Fc region;
wherein the second polypeptide chain comprises a second VD1-(X1)n-VD2-C-(X2)n, wherein
VD1 is a first light chain variable domain;
VD2 is a second light chain variable domain;
C is a light chain constant domain;
X1 is a second linker;
X2 does not comprise an Fc region;
(X1)n is independently (X1)0 or (X1)1 and (X2)n is independently (X2)0 or (X2)1,
wherein the first and second X1 linker are the same or different;
wherein the first X1 linker is not CH1 and/or the second X1 linker is not CL;
wherein the binding protein specifically binds TfR or HIR; and
(a) VD1 or VD2 heavy chain variable domain comprises three CDRs each, wherein at least one of the CDRs comprises an amino acid sequences selected from the group consisting of SEQ ID NO: 76, 77, 78, 82, 83, 115, 116, 117, 156, 157, and 158;
(b) VD1 and VD2 heavy chain variable domains independently comprise three CDRs each comprising amino acid sequences selected from the group consisting of SEQ ID NO: 76, 77, 78, 82, 83, 115, 116, 117, 156, 157, and 158; or
(c) VD1 heavy chain variable domain comprises three CDRs each comprising amino acid sequences selected from the group consisting of SEQ ID NO: 76, 77, 78, 82, 83, 115, 116, 117, 156, 157, and 158, and VD2 heavy chain variable domain comprises three CDRs each comprising amino acid sequences selected from the group consisting of SEQ ID NO: 76, 77, 78, 82, 83, 115, 116, 117, 156, 157, and 158;
and
wherein
(a) VD1 or VD2 light chain variable domain comprises three CDRs each comprising amino acid sequences selected from the group consisting of SEQ ID NO: 79, 80, 81, 84, 85, 86, 118, 119, 120, 159, 160, and 161;
(b) VD1 and VD2 light chain variable domains independently comprise three CDRs each comprising amino acid sequences selected from the group consisting of SEQ ID NO: 79, 80, 81, 84, 85, 86, 118, 119, 120, 159, 160, and 161; or
(c) VD1 light chain variable domain comprises three CDRs each comprising amino acid sequences selected from the group consisting of SEQ ID NO: 79, 80, 81, 84, 85, 86, 118, 119, 120, 159, 160, and 161; and VD2 light chain variable domain comprises three CDRs each comprising amino acid sequences selected from the group consisting of SEQ ID NO: 79, 80, 81, 84, 85, 86, 118, 119, 120, 159, 160, and 161.

23. The binding protein of claim 22, wherein
(a) VD1 or VD2 heavy chain variable domain comprises an amino acid sequence selected from the group consisting of SEQ ID NO: 30, 32, 34, 36, 56, and 104; or
(b) VD1 and VD2 heavy chain variable domains independently comprise an amino acid sequence selected from the group consisting of SEQ ID NO: 30, 32, 34, 36, 56 and 104; and wherein
(a) VD1 or VD2 light chain variable domain comprises an amino acid sequence selected from the group consisting of SEQ ID NO: 31, 33, 35, 37, 57, 105, 106, 107, and 108; or
(b) VD1 and VD2 light chain variable domains independently comprise an amino acid sequence selected from the group consisting of SEQ ID NO: 31, 33, 35, 37, 57, 105, 106, 107, and 108.

24. The binding protein of claim 22, wherein X1 is a peptide linker comprising an at least one amino acid sequence selected from a member of the group consisting of SEQ ID NOs 1-29, 178 and 179.

25-30. (canceled)

31. The binding protein of claim 1 comprising a polypeptide chain, wherein the polypeptide chain comprises VD1-(X1)n-VD2-C-(X2)n, wherein
VD 1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1; and
wherein the binding protein specifically binds to a disease target selected from the group consisting of Abeta, BACE, Her-2, RGMA, TNFα and APP; and
(a) VD1 or VD2 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174;
(b) VD1 and VD2 independently comprise three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174; or
(c) VD1 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174; and VD2 comprises three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174.

32. The binding protein of claim 31, wherein
(a) VD1 or VD2 comprises an amino acid sequence selected from the group consisting of SEQ ID NO: 38, 58, 93, 94, 95, 96, 97, 98, 99, 101, 167, and 169; or
(b) VD1 and VD2 independently comprise an amino acid sequence selected from the group consisting of SEQ ID NO: 38, 58, 93, 94, 95, 96, 97, 98, 99, 101, 162, and 170.

33. The binding protein of claim 1 comprising a polypeptide chain, wherein the polypeptide chain comprises VD1-(X1)n-VD2-C-(X2)n, wherein
VD1 is a first light chain variable domain;
VD2 is a second light chain variable domain;
C is a light chain constant domain;
X1 is a linker with the proviso that it is not CL;
X2 does not comprise an Fc region;
(X1)n is (X1)0 or (X1)1;
(X2)n is (X2)0 or (X2)1; and
wherein the binding protein specifically binds to a disease target selected from the group consisting of Abeta, BACE, Her-2, RGMA, TNFα, and APP; and
(a) VD1 or VD2 comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177;
(b) VD1 and VD2 independently comprise three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177; or
(c) VD1 comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177, and VD2 comprises three each comprising amino acid sequences selected CDRs from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

34. The binding protein of claim 33, wherein
(a) VD1 or VD2 comprises an amino acid sequence selected from the group consisting of SEQ ID NO: 39, 59, 87, 88, 89, 90, 91, 92, 100, 102, 163, and 171; or
(b) VD1 and VD2 independently comprise an amino acid sequence selected from the group consisting of SEQ ID NO: 39, 59, 87, 88, 89, 90, 91, 92, 100, 102, 163, and 171.

35. The binding protein of claim 33, wherein (X1)n is (X1)0.

36. A binding protein comprising first and second polypeptide chains, wherein the first polypeptide chain comprises a first VD1-(X1)n-VD2-C-(X2)n, wherein
VD1 is a first heavy chain variable domain;
VD2 is a second heavy chain variable domain;
C is a heavy chain constant domain;
X1 is a first linker;
X2 is an Fc region;
wherein the second polypeptide chain comprises a second VD1-(X1)n-VD2-C-(X2)n, wherein
VD1 is a first light chain variable domain;
VD2 is a second light chain variable domain;
C is a light chain constant domain;
X1 is a second linker;
X2 does not comprise an Fc region;
(X1)n is independently (X1)0 or (X1)1 and (X2)n is independently (X2)0 or (X2)1,
wherein the first and second X1 linker are the same or different;
wherein the first X1 linker is not CH1 and/or the second X1 linker is not CL;
wherein the binding protein specifically binds to a disease target selected from the group consisting of Abeta, BACE, Her-2, RGMA, TNFα and APP; and
(a) VD1 or VD2 heavy chain variable domain comprises three CDRs each, wherein at least one CDR comprises an amino acid sequence selected from the group consisting of SEQ ID NO: 109, 110, 111, 121, 123, 136, 137, 138, 139, 140, 141, 142, 143, 147, 148, 149, 164, 165, 166, 172, 173, and 174;
(b) VD1 and VD2 heavy chain variable domains independently comprise three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174; or
(c) VD1 heavy chain variable domain comprises three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174; and VD2 heavy chain variable domain comprises three CDRs each comprising amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174;
and
wherein
(a) VD1 or VD2 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152;
(b) VD1 and VD2 light chain variable domains independently comprise three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177; or
(c) VD1 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177, and VD2 light chain variable domain comprises three each comprising amino acid sequences selected CDRs from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

37. The binding protein of claim 36, wherein
(a) VD1 or VD2 heavy chain variable domain comprises an amino acid sequence selected from the group consisting of SEQ ID NO: 38, 58, 93, 94, 95, 96, 97, 98, 99, 101, 162, and 170; or
(b) VD1 and VD2 heavy chain variable domains independently comprise an amino acid sequence selected from the group consisting of SEQ ID NO: 38, 58, 93, 94, 95, 96, 97, 98, 99, 101, 162, and 170;
and wherein
(a) VD1 or VD2 light chain variable domain comprises an amino acid sequence selected from the group consisting of SEQ ID NO: 39, 59, 87, 88, 89, 90, 91, 92, 100, 102, 163, and 171; or
(b) VD1 and VD2 light chain variable domains independently comprise an amino acid sequence selected from the group consisting of SEQ ID NO: 39, 59, 87, 88, 89, 90, 91, 92, 100, 102, 163, and 171.

38-45. (canceled)

46. The binding protein of claim 1 comprising a polypeptide chain, wherein the polypeptide chain comprises VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1; and
wherein the binding protein specifically binds TfR or HIR and Abeta, BACE, Her-2, RGMA, TNFα, or APP;
(a) VD1 or VD2 comprises three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;
(b) VD1 and VD2 independently comprise three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;
(c) VD1 comprises three CDRs each comprising amino acid sequences selected from the group consisting of SEQ ID NO: 76, 77, 78, 82, 83, 115-117 and 156-158, and VD2 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174; or
(d) VD2 comprises three CDRs each comprising amino acid sequences selected from SEQ ID NO: 76, 77, 78, 82, 83, 115-117 and 156-158, and VD1 comprises three CDRs each comprising amino acid sequences selected from the group consisting of SEQ ID NO: 109, 110, 111, 121, 122, 123, 136, 137, 138, 139, 140, 141, 142, 143, 147, 148, 149, 164, 165, 166, 172, 173, and 174.

47. The binding protein of claim 46, wherein
(a) VD1 or VD2 comprises an amino acid sequence selected from the group consisting of SEQ ID NO: 30, 32, 34, 36, 56, 104, 38, 58, 93, 94, 95, 96, 97, 98, 99, 101, 162, and 170; or
(b) VD1 comprises an amino acid sequence selected from the group consisting of SEQ ID NO: 30, 32, 34, 36, 56, or 104 and VD2 comprises an amino acid sequence selected from the group consisting of SEQ ID NO: 38, 58, 93, 94, 95, 96, 97, 98, 99, 101, 162, and 170.

48. The binding protein of claim 1 comprising a polypeptide chain, wherein the polypeptide chain comprises VD1-(X1)n-VD2-C-(X2)n, wherein
VD1 is a first light chain variable domain;
VD2 is a second light chain variable domain;
C is a light chain constant domain;
X1 is a linker with the proviso that it is not CL;
X2 does not comprise an Fc region;
(X1)n is (X1)0 or (X1)1;
(X2)n is (X2)0 or (X2)1; and
wherein the binding protein specifically binds TfR or HIR and Abeta, BACE, Her-2, or APP;
(a) VD1 or VD2 comprises three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;
(b) VD1 and VD2 independently comprise three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;
(c) VD1 comprises three CDRs each comprising amino acid sequences selected from the group consisting of SEQ ID NO: 79, 80, 81, 84, 85, 86, 118, 119, 120, 159, 160, and 161, and VD2 comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152, 167, 168, 169, 175, 176, and 177; or
(d) VD2 comprises three CDRs each comprising amino acid sequences selected from the group consisting of SEQ ID NO: 79, 80, 81, 84, 85, 86, 118, 119, 120, 159, 160, and 161; and VD1 comprises three CDRs each comprising amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

49. The binding protein of claim 48, wherein
(a) VD1 or VD2 comprises an amino acid sequence selected from the group consisting of SEQ ID NO: 31, 33, 35, 37, 57, 105, 106, 107, 108, 39, 59, 87, 88, 89, 90, 91, 92, 100, 102, 163, and 171; or
(b) VD1 comprises an amino acid sequence selected from the group consisting of SEQ ID NO: 31, 33, 35, 37, 57, 105, 106, 107, and 108 and VD2 comprises an amino acid sequence selected from the group consisting of SEQ ID NO: 39, 59, 87, 88, 89, 90, 91, 92, 100, 102, 163, and 171.

50. The binding protein of claim 48, wherein (X1)n is (X1)0.

51. The binding protein of claim 1 comprising first and second polypeptide chains, wherein the first polypeptide chain comprises a first VD1-(X1)n-VD2-C-(X2)n, wherein
VD 1 is a first heavy chain variable domain;
VD2 is a second heavy chain variable domain;
C is a heavy chain constant domain;
X1 is a first linker;
X2 is an Fc region;
wherein the second polypeptide chain comprises a second VD1-(X1)n-VD2-C-(X2)n, wherein
VD1 is a first light chain variable domain;
VD2 is a second light chain variable domain;
C is a light chain constant domain;
X1 is a second linker;
X2 does not comprise an Fc region;
(X1)n is independently (X1)0 or (X1)1 and (X2)n is independently (X2)0 or (X2)1,
wherein the first and second X1 linker are the same or different;
wherein the first X1 linker is not CH1 and/or the second X1 linker is not CL;
wherein the binding protein specifically binds TfR or HIR and Abeta, BACE, Her-2, RGMA, TNFα, or APP;
(a) VD1 or VD2 heavy chain variable domain comprises three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;
(b) VD1 and VD2 heavy chain variable domains independently comprise three CDRs each comprising amino acid sequences selected from the group consisting of SEQ ID NO: 76, 77, 78, 82, 83, 115-117, 156-158, 109, 110, 111, 121, 122, 123, 136, 137, 138, 139, 140, 141, 142, 143, 147, 148, 149, 164, 165, 166, 172, 173, and 174;
(c) VD1 heavy chain variable domain comprises three CDRs each comprising amino acid sequences selected from the group consisting of SEQ ID NO: 76, 77, 78, 82, 83, 115, 116, 117, 156, 157, 158, 164, 165, 166, 172, 173, and 174, and VD2 heavy chain variable domain comprises three CDRs each comprising amino acid sequences selected from the group consisting of SEQ ID NO: 109, 110, 111, 121, 123, 136, 137, 138, 139, 140, 141, 142, 143, 147, 148, and 149; or
(d) VD2 heavy chain variable domain comprises three CDRs each comprising amino acid sequences selected from the group consisting of SEQ ID NO: 76, 77, 78, 82, 83, 115, 116, 117, 156, 157, 158, 164, 165, 166, 172, 173, and 174, and VD1 heavy chain variable domain comprises three CDRs each comprising amino acid sequences selected from the group consisting of SEQ ID NO: 109, 110, 111, 121, 122, 123, 136, 137, 138, 139, 140, 141, 142, 143, 147, 148, and 149;
and
wherein
(a) VD1 or VD2 light chain variable domain comprises three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;
(b) VD1 and VD2 light chain variable domains independently comprise three CDRs each comprising amino acid sequences selected from the group consisting of SEQ ID NO: 79, 80, 81, 84, 85, 86, 118, 119, 120, 159, 160, 161, 112, 113, 114, 124, 125, 126, 127, 128, 129, 130, 131, 132, 133, 134, 135, 150, 151, 152, 167, 168, 169, 175, 176, and 177;
(c) VD1 light chain variable domain comprises three CDRs each comprising amino acid sequences selected from the group consisting of SEQ ID NO: 79, 80, 81, 84, 85, 86, 118, 119, 120, 159, 160, 161, 167, 168, 169, 175, 176, and 177; and VD2 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152; or
(d) VD2 light chain variable domain comprises three CDRs each comprising amino acid sequences selected from the group consisting of SEQ ID NO: 79, 80, 81, 84, 85, 86, 118, 119, 120, 159, 160, 161, 167, 168, 169, 175, 176, and 177; and VD1 light chain variable domain comprises three CDRs each comprising amino acid sequences selected from the group consisting of SEQ ID NO: 112, 113, 114, 124, 125, 126, 127, 128, 129, 130, 131, 132, 133, 134, 135, 150, 151, and 152.

52. The binding protein of claim 51, wherein
(a) VD1 or VD2 heavy chain variable domain comprises an amino acid sequence selected from the group consisting of SEQ ID NO: 30, 32, 34, 36, 56, 104, 38, 58, 93, 94, 95, 96, 97, 98, 99, 101, -162, and 170; or
(b) VD1 heavy chain variable domain comprises an amino acid sequence selected from the group consisting of SEQ ID NO: 30, 32, 34, 36, 56, or 104 and VD2 heavy chain variable domain comprises an amino acid sequence selected from the group consisting of SEQ ID NO: 38, 58, 93, 94, 95, 96, 97, 98, 99, 101, 162, and 170;
and wherein
(a) VD1 or VD2 light chain variable domain comprises an amino acid sequence selected from the group consisting of SEQ ID NO: 31, 33, 35, 37, 57, 105, 106, 107, 108, 39, 59, 87, 88, 89, 90, 91, 92, 100, 102, 163, and 171; or
(b) VD1 light chain variable domain comprises an amino acid sequence selected from the group consisting of SEQ ID NO: 31, 33, 35, 37, 57, 105, 106, 107, or 108 and VD2 light chain variable domain comprises an amino acid sequence selected from the group consisting of SEQ ID NO: 39, 59, 87, 88, 89, 90, 91, 92, 100, or 102, 163, and 171.

53-59. (canceled)

60. A monospecific binding protein comprising
a heavy polypeptide chain of comprising an amino acid sequence selected from the group consisting SEQ ID NO: 76, 77, 78, 82, 83, 115-117 and 156-158, and a light polypeptide chain comprising an amino acid sequence selected from the group consisting of SEQ ID NO: 79, 80, 81, 84, 85, 86, 118, 119, 120, 159, 160 and 161; or
a heavy polypeptide chain comprising an amino acid sequences selected from the group consisting of SEQ ID NO: 109, 110, 111, 121, 123, 136, 137, 138, 139, 140, 141, 142, 143, 147, 148, 149, 164, 165, 166, 172, 173, and 174, and a light polypeptide chain comprising an amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177.

61. A bispecific binding protein comprising
a heavy polypeptide chain comprising an amino acid sequence selected from the group consisting SEQ ID NO: 76, 77, 78, 82, 83, 115-117 and 156-158, and a light polypeptide chain comprising an amino acid sequences selected from the group consisting of SEQ ID NO: 112, 113, 114, 124, 125, 126, 127, 128, 129, 130, 131, 132, 133, 134, 135, 150, 151, 152, 167, 168, 169, 175, 176, and 177;
a heavy polypeptide chain comprising an amino acid sequence selected from the group consisting of SEQ ID NO: 109, 110, 111, 121, 123, 136, 137, 138, 139, 140, 141, 142, 143, 147, 148, 149, 164, 165, 166, 172, 173, and 174, and a light polypeptide chain comprising an amino acid sequence selected from the group consisting of SEQ ID NO: 79, 80, 81, 84, 85, 86, 118, 119, 120, 159, 160, and 161;
a heavy polypeptide chain comprising an amino acid sequence selected from the group 121, 122, 123, 136, 137, 138, 139, 140, 141, 142, 143, 147, 148, 149, 156-158, 164, 165, 166, 172, 173, and 174, and a light polypeptide chain comprising an amino acid sequence selected from the group consisting of SEQ ID NO: 79, 80, 81, 84, 85, 86, 112, 113, 114, 118, 119, 120, 161, 124, 125, 126, 127, 128, 129, 130, 131, 132, 133, 134, 135, 150, 151, 152, 159, 160, 167, 168, 169, 175, 176, and 177; or
a heavy polypeptide chain comprising an amino acid sequence selected from the group consisting of SEQ ID NO: 76, 77, 78, 82, 83, 109, 110, 111, 115-117, 121, 122, 123, 136, 137, 138, 139, 140, 141, 142, 143, 147, 148, 149,156-158, 164, 165, 166, 172, 173, and 174, or comprising an amino acid sequence selected from the group consisting of SEQ ID NO: 109, 110, 111, 121, 123, 136, 137, 138, 139, 140, 141, 142, 143, 147, 148, 149, 164, 165, 166, 172, 173, and 174, and a light polypeptide chain comprising an amino acid sequence selected from the group consisting SEQ ID NO: 79, 80, 81, 84, 85, 86, 112, 113, 114, 118, 119, 120, 161, 124, 125, 126, 127, 128, 129, 130, 131, 132, 133, 134, 135, 150, 151, 152, 159, 160, 167, 168, 169, 175, 176, and 177.

62. The binding protein of claim 1, wherein the binding protein comprises Out1-(X1)m-In1-(X2)n, wherein In1 specifically binds to the antigen expressed on the brain vascular epithelium of the subject, wherein Out1 specifically binds to another molecule, wherein X1 is a linker, wherein X2 is an Fc region, wherein m is 0 or 1 and wherein n is 0 or 1.

63-65. (canceled)

66. The binding protein of claim 64, wherein In1 comprises the amino sequence of SEQ ID NO:56.

67-69. (canceled)

70. The binding protein of claim 69, wherein Out1 specifically binds transferrin receptor.

72. The binding protein of claim 64, wherein Out1 comprises the amino sequence of SEQ ID NO:36.

73-82. (canceled)

83. An isolated nucleic acid encoding the binding protein amino acid sequence of claim 1.

84-89. (canceled)

90. A method of producing a binding protein, comprising culturing a host cell described in any one of claim 86 in culture medium under conditions sufficient to produce the binding protein.

91. (canceled)

92. A pharmaceutical composition comprising the binding protein of claim 1, and a pharmaceutically acceptable carrier.

93-117. (canceled)

118. A humanized antibody that specifically binds:
TfR comprising an amino acid sequence selected from the group consisting of SEQ ID NO: 30-37, 56 and 57; or,
HIR comprising an amino acid sequence selected from the group consisting of SEQ ID NO: 104-108.

119-120. (canceled)
